(19) United States
(12) Patent Application Publication
(10) Pub. No.: US 2002/0127623 A1
(43) Pub. Date: Sep. 12, 2002

(54) Biosensors, reagents and diagnostic applications of directed evolution

(75) Inventors: Jeremy Minshull, Menlo Park, CA (US); S. Christopher Davis, San Francisco, CA (US); Mark Welch, Fremont, CA (US); Sun Ai Raillard, Mountain View, CA (US); Kurt Vogel, Palo Alto, CA (US); Claus Krebber, Mountain View, CA (US)

Correspondence Address: LAW OFFICES OF JONATHAN ALAN QUINE, P O BOX 458, ALAMEDA, CA, 94501

(73) Assignee: Maxygen, Inc., Redwood City, CA

(21) Appl. No.: 09/920,607

(22) Filed: Jul. 31, 2001

Related U.S. Application Data
(60) Provisional application No. 60/222,056, filed on Jul. 31, 2000.
(60) Provisional application No. 60/244,764, filed on Oct. 31, 2000.

(51) Int. Cl. G01N 33/53; G01N 33/537; G01N 33/543

(52) U.S. Cl. 435/7.92; 435/7.1

(57) ABSTRACT

Methods for sensing test stimuli using arrays of biopolymers are provided. Libraries of biopolymers, such nucleic acid variants, and expression products encoded by nucleic acid variants are provided. Reusable library arrays, and methods for their use are provided.

### CROSS REFERENCE TO RELATED APPLICATIONS

This application claims priority to and benefit of U.S. Provisional Applications No. 60/222,056, filed Jul. 31, 2000, and Provisional Application No. 60/244,764, filed Oct. 31, 2000, the disclosures of each of which are incorporated herein in their entirety for all purposes.

### FIELD OF THE INVENTION

The present invention relates to biosensors comprising diversified components, automated devices and systems for using arrays of diversified (e.g., shuffled) nucleic acids, and diverse encoded products, e.g., as bar-code systems for screening libraries, identifying compounds, and the like. The biosensors and arrays are typically provided in a re-usable format, providing new types of general laboratory tools. The biosensors can take any of a variety of forms, including conformation-sensitive polymers.

### BACKGROUND OF THE INVENTION

Today's laboratory is focused in part on the dramatically increasing need for analytical data brought about by the increased pace of new product development, increased research, demands for stricter quality control, and the like. Labs deliver data in a timely, cost-efficient way while ensuring precise results, clear documentation, and minimal use of skilled (and, therefore, expensive) personnel. For example, automated systems have been proposed to assess a variety of biological phenomena, including, e.g., expression levels of genes in response to selected stimuli (Service (1998) “Microchips Arrays Put DNA on the Spot” Science 282:396-399), high throughput DNA genotyping (Zhang et al. (1999) “Automated and Integrated System for High-Throughput DNA Genotyping Directly from Blood” Anal. Chem. 71:1138-1145) and many others.

One general example of laboratory tools utlizes arrays of biopolymers, such as arrays of nucleic acids or proteins. For example, companies such as Affymetrix (e.g., VLSIPS® arrays; Santa Clara, Calif.), Hyseq (Mountain View, Calif.), Research Genetics (e.g., the GeneFilters® microarrays; Huntsville Ala.), Axon Instruments (GenePix®; Foster City, Calif.), Operon (e.g., OpArrays®, Alameda, Calif.) and others provide many technologies for making physical arrays of nucleic acids and other molecules. For example, arrays have been used for Disease Management issues, Expression Analysis, GeneChip Probe Array Technologies, Genotyping and Polymorphism analysis, Spotted Array Technologies and the like. For a list of publications related to the topic of array construction and use, see, www.affymetrix.com/resources/scientific paper.html and www.hyseq.com/company/cbibl.html. Reviews of nucleic acid arrays include Sapolsky et al. (1999) “High-throughput polymorphism screening and genotyping with high-density oligonucleotide arrays.” Genetic Analysis: Biomolecular Engineering 14:187-192; Lockhart (1998) “Mutant yeast on drugs” Nature Medicine 4:1235-1236; Fodor (1997) “Genes, Chips and the Human Genome.” FASEB Journal 11 :A879; Fodor (1997) “Massively Parallel Genomics.” Science 277: 393-395; and Chee et al. (1996) “Accessing Genetic Information with High-Density DNA Arrays.” Science 274:610-614.

Examples of protein-based arrays include immuno arrays (see, e.g., http://arrayit.com/protein-arrays/; Holt et al. (2000) “By-passing selection: direct screening for antibody-antigen interactions using protein arrays.” Nucleic Acids Research 28(15) E72-e72), superproteins arrays (see, e.g., www.jst.go.jp/erato/project/nts P/nts P.html), yeast two and other “n” hybrid array systems (see, e.g. Uetz et al. (2000) “A comprehensive analysis of protein-protein interactions in Saccharomyces cerevisiae” Nature 403, 623-627, and Vidal and Legrain (1999) “Yeast forward and reverse ‘n’-hybrid systems.” Nucleic Acids Research 27(4) 919-929); the universal protein array or “LPA” system (Ge et al. (2000) “UPA, a universal protein array system for quantitative detection of protein-protein, protein-DNA, protein-RNA and protein-ligand interactions.” Nucleic Acids Research, 28(2): E3-e3) and the like. Commercial companies such as Ciphergen (Freemont, Calif.); www.ciphergen.com, Beckman Coulter Inc. (Brea, Calif.); and others also provide commercial protein chip arrays.

In addition to arraying materials, laboratory systems can also perform, e.g., repetitive fluid handling operations (e.g., pipetting) for transferring material to or from reagent storage systems that comprise arrays, such as microtiter trays or other chip trays, which are used as basic container elements for a variety of automated laboratory methods. Similarly, the systems manipulate, e.g., microtiter trays and control a variety of environmental conditions such as temperature, exposure to light or air, and the like.

Many such automated systems are commercially available. For example, a variety of automated systems are available from the Zymark Corporation (Zymark Center, Hopkinton, Mass.), which utilize various Zymate systems (see also, www.zymark.com/), which typically include, e.g., robotics and fluid handling modules. Similarly, the common ORCA® robot, which is used in a variety of laboratory systems, e.g., for microtiter tray manipulation, is also commercially available, e.g., from Beckman Coulter, Inc. (Fullerton, Calif.).

Although array systems are widely available, and in use for many diverse applications, including high-throughput applications, additional ways of constructing and using arrays would be desirable. The present invention provides many new array constructs, including high throughput embodiments, e.g., using shuffling methods to create arrays of interest. These arrays are useful as commercial and laboratory tools in a variety of settings, as discussed in detail herein.

### SUMMARY OF THE INVENTION

The present invention provides novel methods for detecting a wide range of biological, chemical and biochemical stimuli. The methods of the invention utilize biopolymers and arrayed libraries of biopolymers, members of which are capable of binding the biological, chemical or biochemical stimuli, and upon binding produce a detectable signal.

In a first aspect, the invention provides methods for detecting a wide variety of analytes, such as small organic molecule, an ion, a polypeptide or peptide, a gas, a dissolved gas (e.g., O₂), an inorganic molecule, or a metabolite. For example, the invention provides methods for detecting an analyte involving providing biopolymers, including nucleic acids and proteins, such as enzymes, fluorescent proteins, receptors, and antibodies, that undergo conformational changes upon binding to an analyte. In some embodiments, methods for identifying physiologic states are provided, wherein a conformational change resulting in a detectable signal is produced upon binding of a marker associated with a physiological state, such as a disease.

In preferred embodiments, the analytes are non-nucleic acid analytes, in particular small molecule analytes. For example, the methods involve providing at least one fusion polypeptide specific for a non-nucleic acid analyte having a first inactive functional domain; an analyte binding domain; and a second inactive functional domain. The fusion polypeptides are designed or selected such that analyte binding results in a conformational change which brings the first inactive functional domain and the second inactive functional domain into proximity, thereby converting the first and second inactive functional domains into an optically detectable functional domain. For example, the first and second inactive functional domains can be derived from green fluorescent protein (GFP) or a GFP homologue. The fusion polypeptide(s) is contacted with a sample, such as a biological or environmental sample (e.g., blood, plasma, urine, sweat, cerebrospinal fluid, tears) containing the analyte, and a signal dependent on the conformational change induced by analyte binding is detected. In preferred embodiments, the non-nucleic acid analyte is a small organic molecule, or a metabolite.

In other embodiments, a fusion polypeptide having a first inactive functional domain; an analyte binding domain; and a second inactive functional domain, that are brought into proximity to form a functional catalytic domain upon binding of a non-nucleic acid analyte are provided. In this case, a substrate is provided, and upon binding of an analyte, such as a small molecule, e.g., a hormone, a metabolite, or an ion, is converted to a detectable product to produce a signal.

In yet other embodiments, the methods involve a polypeptide with specificity for a non-nucleic acid analyte having an analyte binding domain and a catalytic domain which is activated by an allosteric conformational change induced by binding of the analyte, such as a hormone, metabolite, ion, antigen, ligand, agonist or antagonist.

In some embodiments, the signal is an electrochemical signal, in other embodiments, the signal is an optical signal detected by ultraviolet spectrophotometry, visible light spectrophotometry, surface plasmon resonance, calorimetry, fluorescence polarization, fluorescence quenching, calorimetric quenching, fluorescence wavelength shift, fluroescence resonance energy transfer (FRET), enzyme inked immunosorbent assay (ELISA), liquid crystal displays (LCD) or a charge coupled device. In certain embodiments, binding of an analyte produces an optical signal by displacing a tethered substrate, such as an analyte analogue.

In certain embodiments, a plurality of polypeptides, such as fusion polypeptides, enzymes that are activated by a conformational change, etc., are provided, e.g., in a physical or logical array. In some embodiments, the plurality of polypeptides include polypeptides with different analyte binding specificities. In preferred embodiments, the plurality of polypeptides yield a common signal or read-out, that is a signal that is detectable by a common detection method or device, e.g., on a common detection platform.

The polypeptides, fusion polypeptides, and arrays of such polypeptides including a plurality of polypeptides or fusion polypeptides with identical, overlapping or different analyte specificities can be used as biosensors, for example, by immobilizing (e.g., using a carbon paste, a non-biological polymer, and other immobilization methods that are well known in the art) the polypeptide or plurality of polypeptides on a support, and optionally, coupling the support to a detector system. The biosensor polypeptides can, thus, be used to produce biosensor devices, for example hand-held or implantable biosensor devices for detecting one or more stimuli in a biological or environmental sample. If desired, the devices can also include a display, such as an optical or digital display.

In some embodiments, the libraries of biopolymers are deoxyribonucleic acid (DNA) variants. In alternative embodiments, the libraries of biopolymers are RNA or protein expression products of the DNA variants. The libraries are arrayed in a spatial or logical format to provide a spatial or logical library array. After calibrating the array with one or more calibrating stimulus that results in a calibrating array pattern associated with the stimulus or stimuli, the library array is exposed to one or a battery of test stimuli. Upon contact with the test stimulus, a test stimulus array pattern is produced and detected. The test stimulus array pattern is then compared to the calibrating array pattern enabling identification of the test stimulus. In one general aspect, the present invention provides biosensors of diversified materials, whether arrayed or not.

In some embodiments, the array libraries are reusable. Methods for making and using a re-usable array of biopolymers involve, e.g., providing a library of biopolymers, arraying the library to physical or logical format, exposing the arrayed library with one or more first stimulus and observing a first response or collecting a first product resulting from contact between the array and the first stimulus, then reusing the array by exposing the array to the same or a different stimulus, and again observing the response or collecting a product resulting from contact between the array and the stimulus. Optionally, the first and subsequent results or products are compared, e.g., to identify the first or subsequent stimuli.

In some embodiments, the library is composed of, or encoded by, recombinant nucleic acids produced by directed evolution, e.g., nucleic acids that are recursively recombined, e.g., shuffled. In some embodiments, the library is composed of, or encoded by, nucleic acids which have been mutated or recombined through artificial processes, e.g., shuffled. In some instances, the library is made up of species variants of one or more nucleic acids or expression products. Optionally, the library is produced by recursive recombination of species variants of one or more nucleic acids.

In some embodiments, the biopolymer library is made up of photoactive or photoactivatable members. Optionally, a portion of such an array is masked, and the array exposed to light to activate some or all of the members of the library.

Optionally, the biopolymer library includes one or more members that are conductive, capacitative, optically responsive, electrically responsive, or electrically or logically gated or gateable. Examples include libraries having members that are bio-lasers, polychromic displays, molecular posters, bar codes, protein TVs, molecular cameras, UV molecular cameras, IR molecular cameras, and flat screen displays.

In some embodiments, the biopolymers of the array include proteins. In one embodiment, the proteins are electrically conductive proteins. Optionally, the proteins of the libraries are purified. To facilitate purification, the proteins, optionally, include purification tags such as His tags and FLAG tags. Other epitope or purification tags are also suitable. Optionally, the members of the library are selected, prior to assembly into arrays, for one or more of: enhanced stability, orientation of protein binding, improved production, cost of manufacture, optimal activity of expressed members which comprise a tag, overexpression mutations, optimized protein folding, permanent enzyme secretion, improved operators, improved ribosome binding sites, avidity, selectivity, production of a detectable side product, and detection limit.

The libraries are assembled into arrays by arranging the members of the library in a logically accessible format or in a physically gridded format. This can be accomplished, for example, by depositing the members of the library in microtiter trays, e.g., by plating cells incorporating DNA variants or expressing RNAs or proteins encoded by the DNA variants.

Alternatively, or in tandem, the positions of members of the library are recorded in one or more database. The arrays of the invention can be arranged for either (or both) parallel examination or sequential examination. For example, any of the stimuli, e.g., the first, second, test or calibrating stimulus, can be simultaneously or sequentially contacted to arrayed members of the biopolymer library. Optionally, multiple stimuli, e.g., first, second, test or calibrating stimuli, are contacted to the arrayed members of the biopolymer library. For example, one or more stimuli can be contacted to library members in microtiter plates or fixed on a solid substrate, e.g., a Nickel-NTA coated surface, a silane-treated surface, a pegylated surface, or a treated surface. Alternatively, one or more stimuli can be contacted to library members fixed to an organizational matrix in spatially addressable locations.

In other embodiments, one or more stimuli are contacted to library members fixed on the surface of beads. Each bead, optionally, includes more than one detectable feature, e.g., a feature that identifies binding by a stimulus, and a feature that identifies either the type of bead or the type of library member bound to the bead.

In yet other embodiments, one or more stimuli are contacted to library members by incubating a solution containing the stimulus with one or more library members. The solution can be, e.g., a fluid, an extract, a polymer solution or a gel.

In the methods of the invention, a stimulus, such as a first, second, test or calibrating stimulus, is optionally selected from among light, radiation, atoms, ions and molecules. Such a stimuli can comprise, hybridize, act upon or be acted upon by one or more of: radiation (e.g., visible light radiation, uv radiation, isotopic or non-isotopic radiation, fluorescence, etc.), a polymer, a biopolymer, a nucleic acid, an RNA, a DNA, a protein, a ligand, an enzyme, a chemo-specific enzyme, a regio-specific enzyme, a stereo-specific enzyme, a nuclease, a restriction enzyme, a restriction enzyme which recognizes a triplet repeat, a restriction enzyme that recognizes DNA superstructure, a restriction enzyme with an 8 base recognition sequence, an enzyme substrate, a regio-specific enzyme substrate, a stereo-specific enzyme substrate, a ligase, a thermostable ligase, a polymerase, a thermostable polymerase, a lipase, a protease, a glycosidase, a chemical moiety, a co-factor, a toxin, a contaminant, a metal, a heavy metal, an immunogen, an antibody, a disease marker, a cell, a tumor cell, a tissue-type, cerebro-spinal fluid, a cytokine, a receptor, a chemical agent, a biological agent, an airborne stimulus, an odor (e.g., a fragrance), a pheromone, a hormone, an olfactory protein, a metabolite, a molecular camera protein, a rod protein, a cone protein, a light-sensitive protein, a lipid, a pegylated material, an adhesion amplifier, a drug, a potential drug, a lead compound, a protein allele, an oxidase, a reductase, or a catalyst.

Contact of a stimulus, e.g., a first, a second, a test, or a calibrating stimulus, to the array results in the production of a corresponding array pattern, response, or product. In some cases, contact of one of the above stimulus, or types of stimuli, e.g., first, second, test or calibrating, produces a signature for a sample type. Such a signature is representative, e.g., of one or more phenomena selected from: a metabolic state of a cell, an operon induction in or by a cell, an induction of cell growth, a proliferation in or caused by a cell, a cancer of a cell or tissue, or organism, apoptosis, cell death, cell cycle, cell or tissue differentiation, tumorigenesis, disease state, drug resistance, drug efficacy, antibiotic spectrum, drug toxicity, gas level, SOₓ, NOₓ, disease state, physiological status, e.g., neurological status with respect to a specified disagnosis such as Alzheimer's disease, infection, presence of viruses, viral infection, bacterial infection, HIV infection, AIDS, blood glucose level, ion or gas production or internalization, serum cholesterol, CHDL level, LDL, serum triglyceride level, cytokine receptor expression, antibody-antigen interactions, pregnancy, fertility, fecundity, presence or absence of narcotics or other controlled substances, cardiovascular status, e.g., occurrence or predisposition to myocardial infarction (heart attack), congestive heart failure, etc., presence or absence of steroids, body temperature, presence of sound waves, taste, odor, scent, food composition, beverage composition, and an environmentally monitored condition.

In some embodiments, one or more array pattern or response is digitized and stored in a database in a computer. In some embodiments, a comparison of patterns or responses resulting from contact of stimuli, e.g., test and calibrating stimuli or first and second stimuli, to the array is performed by a computer. Optionally, a plurality of stimuli, e.g., first, second, test or calibrating stimuli, are contacted to the array to produce a plurality of resulting array patterns or responses. Optionally, the plurality of array patterns or responses is recorded in a database. In some cases, a bar code is assigned to each resulting array pattern or response. Such databases, the data sets they represent, and computers including data sets corresponding to the array patterns and/or responses resulting from contacting a stimulus with the arrays of the invention, are also a feature of the invention.

In some embodiments, the array patterns and/or the responses resulting from contacing the stimulus with the array, include variations in the presence or absence of signal at different locations on or in the array. Alternatively, the array patterns and/or responses include variations in the level of signal at different locations on the array. In some cases, the array patterns and/or responses include variations in both the presence and the intensity of signal at different locations on the array. Optionally, the intensity of the array pattern and/or response is measured to quantify the corresponding stimulus.

In some embodiments, the array pattern or the resulting response includes one or more fluorophore emission, photon emission, chemiluminescent emission, coupled luminescent/fluorescent emission or quenching, or detection of a fluorophore emission. For example, the array pattern or response is made up of a fluorophore emission generated by light, H₂O₂, glucose oxidase, NADP, NADPH⁺, NAD(P)H reductase, an electorchemally detectable signal, an amperometrically detectable signal, a potentiometrically detectable signal, a signal detectable as a change in pH, a signal based on specific ion levels, a signal based on changes in conductivity, a pizoelectric signal, a change in resonance frequency, a signal detectable as surface accoustic waves, or a signal detectable by quartz crystal microbalances, a reduction potential, a protein conformational change, a intrinsic fluorescence, fluorescence, luminescence, FRET, absorption, surface plasmon resonance, antigen binding, antibody binding, enzyme activity, opening of an ion channel, or label binding. Optionally, the array pattern or response is a complex optical signal encompassing multiple wavelengths of light.

Any of these array patterns or responses are optionally detected by a microscope, a CCD, a phototube, a photodiode, an LCD, a scintillation counter, film, or visual inspection.

Biopolymer arrays, such as the arrayed libraries of nucleic acid variants and their expression products, produced by the methods of the invention are a feature of the invention. In some embodiments, the arrays are stable under normal storage and use conditions. For example, the arrays can be stable for at least one year under pre-selected storage conditions.

### BRIEF DESCRIPTION OF THE FIGURES

FIG. 1 schematically illustrates a common signal transduction platform for detecting metabolites.

FIG. 2 schematically illustrates a multi-analyte detector.

FIG. 3 schematically illustrates an electrically coupled biosensor.

FIG. 4 schematically illustrates an exemplary device platform.

FIG. 5 schematically illustrates detection of an anlayte using a tethered FRET substrate. A. shows the coformation of a labeled analogue bound in the absence of analyte. B. shows the conformational shift induced upon binding of the analyte.

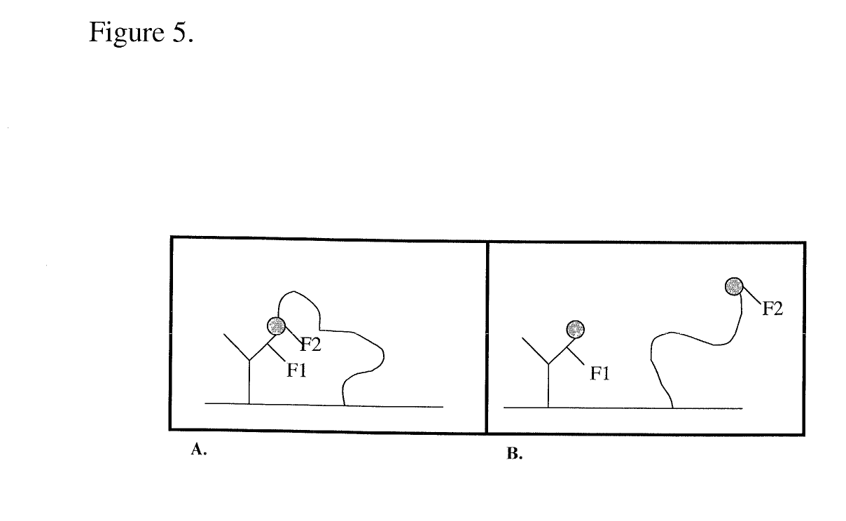

FIG. 6 illustrates activity of twelve triazine hydrolase enzyme variants towards six different substrates.

FIG. 7 schematically illustrates the catalytic activity of xanthine oxidase toward theophylline and three related substrates

### DETAILED DISCUSSION

The development of biosensors involves the identification and optimization of biopolymers that interact in a specific manner with one or more analytes, and then translate that specific interaction into the generation of a detectable signal. The present invention relates to the production of analyte specific biopolymers, as well as methods for using these biopolymers to generate signals detectable, typically by electrical, electrochemical, or optical means suited for employment in sensing devices.

Biosensors of the invention are used as monofunctional detectors or as multianalyte sensors. Typically, the latter involve arrays of biopolymers that serve as biosensors. The present invention also provides novel detection methods for use in monofunctional and multifunctional biosensor devices, as well as exemplary devices, which can be multifunctional or dedicated to detection of a single analyte. Such biosensors have widespread applicability in medical and environmental monitoring, as well as numerous other research and commercial applications.

The present invention provides several new biopolymer biosensors, array formats, including biosensors, physical and logical biopolymer arrays (including biosensor arrays), biopolymer arrays for production or identification of compounds, and the like. These biosensors and arrays are useful, e.g., as sensor arrays, for such applications as metabolic profiling, toxicology, drug discovery, biomarker detection, catalyst library screening, environmental monitoring, process control, and for use as molecular computers, as well as many other uses that will become apparent upon further review. In another aspect, the invention provides biosensors comprising diversified (e.g., shuffled) biopolymer components. In addition, the invention provides detection methods which increase opportunities for biosensor development, and platforms and devices for employing biosensors.

It will be noted that while discussion of specific aspects of the invention focuses on either single biosensor biopolymers or on arrays of biopolymers, unless the context indicates one or the other to be exclusive, the methods and devices described are applicable to both single biosensor molecules and biosensor arrays.

Definitions

A “biopolymer” is a biological macromolecule made up of identifiable subunits. Examples of biopolymers include: nucleic acids, e.g., DNA, RNA and known variants thereof such as PNAs; polypeptides, including proteins (including modified proteins such as glycoproteins, PEGylated proteins, etc.); complex carbohydrates, e.g., starches; lipids, combinations thereof, etc. A “library of biopolymers” or “biopolymer library” is a collection of at least two, typically more than about 10, more typically more than about 50, often more than about 100, and frequently more than about 500, or about 1000, or more biopolymer types. The biopolymer libraries of the invention can include a diverse set of related nucleic acids or nucleic acid “variants.” Alternatively, the biopolymer libraries of the invention include a diverse set of expression products, most typically, protein (or polpeptide) variants encoded by a library of DNA variants. In some cases, the variants are cognates, or orthologues, of a nucleic acid or protein from different species, i.e., “species variants.”

A “peptide” is a polymer of amino acid residues comprising a length of between about 2 and 50 amino acid residues, or of between about 2 and 20 amino acid residues, or of between about 2 and 10 residues. A “polypeptide” is a polymer of amino acid residues typically comprising a length of greater than 50 amino acid residues.

The term “member,” when referring to a library, e.g., of biopolymers, is used to refer to a single constituent, or component, biopolymer in the library, or, alternately, depending on the context, to refer to a type of component at an array location (it will be appreciated that many individual biopolymers can be located in a region of an array which defines an array position). As such, a member of a library can be a DNA variant, or an RNA or protein expression product encoded by a DNA variant, or a class of essentially similar members in a specified array location.

“Arraying” refers to the act of organizing or arranging members of a library, or other collection, into a logical or physical array.

An “array” refers to a physical or logical arrangement of, e.g., library members. A physical array can be any “spatial format” or “physically gridded format” in which physical manifestations of individual library members are arranged in an ordered manner. For example, isolated DNA samples corresponding to individual or pooled members of a library can be arranged in a series of numbered rows and columns, e.g., on a filter, membrane or series of pins or beads. Similarly, transformed cells incorporating library members can be plated or otherwise deposited in microtiter, e.g., 96 well, 384 well or 1536 well, plates (or trays).

Alternatively, an array can be a logical array, i.e., any “logical format” or “logically accessible format,” such as a data set correlating locations of physical samples, with accessible identification desginations, such as “spatially addressable locations.” Most typically, data sets of this nature are stored and accessed in a computer readable medium and/or in a computer.

Thus, as noted herein, the arrays of the invention can be, and often are, physical arrays, but can also be logical arrays. A “physical array” is a set of specified elements arranged in a specified or specifiable spatial arrangement (e.g., as in a solid-phase or “chip” array, a microtiter arrangement, or the like. A “logical array” is a set of specified elements arranged in a manner which permits access to the elements of the set. A logical array can be, e.g., a virtual arrangement of the set in a computer system, or e.g., an arrangement of set elements produced by performing a specified physical manipulation on one or more set element or components of set elements. For example, a logical array can be described in which set elements (or components that can be combined to produce set elements) can be transported or manipulated to produce the set. A “duplicate” or “copy” array is an array which can be at least partially corresponded to a parental array. In simplest form, this correspondence takes the form of simply replicating all or part of the parental array, e.g., by taking an aliquot of material from each position in the parental array and placing the aliquot in a defined position in the duplicate array. However, any method which results in the ability to correspond members of the duplicate array to the parental array can be used for array duplication, including the use of complex storage algorithms, partially or purely in silico arrays, and pooling approaches which partially combine some elements of the parental array into single locations (physical or virtual) in the duplicate array. The duplicate or copy array duplicates some or all components of a parental array. For example, an array of reaction mixtures might include nucleic acids and translation or transcription reagents at sites in the array, while the duplicate/copy array can also include the complete reaction mixtures, or, alternately, can include, e.g., the nucleic acids, without the other reaction mixture components.

A “solid phase array” is a physical array in which the members of the array are fixed to a solid substrate. The fixation can be the result of any interaction that tends to immobilize components, including chemical linking, heat treatment, physical entrapment, encapsulating, or the like. A “solid substrate” has a fixed organizational support matrix, such as silica, polymeric materials, membranes, beads, pins, glass, etc. In some embodiments, at least one surface of the substrate is partially planar, but in others, the solid substrate is a discrete element such as a bead which can be dispensed into an organization matrix such as a microtiter tray. Solid support materials include, but are not limited to, glass, polacryloylmorpholide, silica, controlled pore glass (CPG), polystyrene, polystyrene/latex, polyethylene, polyamide, carboxyl modified teflon, nylon and nitrocellulose and metals and alloys such as gold, platinum and palladium. The solid substrates can be biological, nonbiological, organic, inorganic, or a combination of any of these, existing as particles, strands, precipitates, gels, sheets, tubing, spheres, containers, capillaries, pads, slices, films, plates, slides, etc., depending upon the particular application. Other suitable solid substrate materials will be readily apparent to those of skill in the art. Often, the surface of the solid substrate will contain reactive groups, such as carboxyl, amino, hydroxyl, thiol, or the like for the attachement of nucleic acids, proteins, etc. Surfaces on the solid substrate will sometimes, though not always, be composed of the same material as the substrate. Thus, the surface can be composed of any of a wide variety of materials, for example, polymers, plastics, resins, polysaccharides, silica or silica-based materials, carbon, metals, inorganic glasses, membranes, or any of the above-listed substrate materials.

A “liquid phase array” is an array in which the members of the array are free in solution, e.g., on a microtiter tray, or in a series of containers (such as a set of test tubes or other containers).

A “mediator” is an electrochemically active species (typically, though not exclusively, a small molecule such as ferricyanide, ferrocene and the like), which is capable of transferring electrons between the biopolymer and the electrode of the sensor.

A “metabolite,” as used herein, is a substance involved in metabolism, being either produced during metabolism or taken in from the environment, such as a metabolic product, intermediate, or by-product.

The term “calibrating stimulus” or “pattern forming stimulus” refers to a known stimulus, that elicits a measurable response upon contact with one or more members of a biopolymer library. The response elicited from the collective of members of an array by a calibrating stimulus is designated a “calibrating array pattern” or a “labeling array pattern.” A “test stimulus” is a stimulus, typically, of an unknown composition or origin. The response elicited upon contact of the test stimulus is designated a “test stimulus array pattern,” and is reflective of a measurable pattern of responses elicited by the test stimulus from members of the library. For example, identity between a test stimulus array pattern and a calibrating array pattern from a single array is indicative of identity between the calibrating stimulus and the test stimulus, i.e., the control sample and the test sample are the same compound.

The collective responses elicited by a stimulus from an array is termed its “signature” or “fingerprint.”

Biosensor Platform

Prior to the present invention, two factors have prevented the development of a multi-analyte biosensor. The first is the diversity of analyte specificities of available natural enzymes. The second is the ability of these naturally occurring enzymes to function on a surface amenable to generation and detection of a signal, e.g., an electrode. Most efforts in producing enzyme-based detectors have previously been directed towards engineering and enzyme formulation solutions to these problems. For example, twenty years of engineering efforts have been devoted to constructing a glucose biosensor for diabetes patients, even though glucose oxidase, the enzyme used in the glucose biosensor, is fortuitously a very robust enzyme compared to most natural enzymes.

Re-engineering costs for the creation of a completely new device for each individual enzyme are prohibitive. The present invention approaches these limitations from a biological perspective, using directed evolution, e.g., DNA shuffling and other procedures as described hereinbelow to produce a diversity of biopolymers capable of functioning as sensors on a standardized signal detection platform, such as the glucose oxidase sensor and platform, or other existing formulation and engineering platforms, allowing cost-effective mass manufacture of the device. While oxidases are used in preferred embodiments of the current invention, it will be appreciated that any of a variety of sets of proteins can be adapted to the common signal transduction platforms, etc. herein. Naturally occurring biopolymers, such as enzymes, antibodies, lipocalins,anticalins,and receptors with the desired specificity are evolved to the desired sensitivity and to function on the selected platform. For the sake of brevity, the descriptions herein typically describe use of antibodies in the current methods/devices, however, it is to be understood that lipocalins, anticalins, and any other group or family of protein(s) comprising specific binding domains and/or binding areas are optionally used. Alternatively, novel biopolymers with the desired characteristics, e.g., fusion proteins having analyte binding and signal generation domains, are produced.

In one aspect, the biosensor platform suitable for use in a hand-held dedicated or multi-analyte detection device. The present invention provides that the device can be remotely linked to computational facilities for data analysis. For example, enzyme-based electrical signal generation can be performed on crude biological samples without requiring extensive sample preparation. This represents a considerable advantage over alternative technologies, in that it obviates the need for trained laboratory personnel.

In a multi-analyte sensor of the present invention, although the different biosensor molecules recognize different analytes (e.g., metabolites), the result of each biosensor-analyte binding and signaling event is typically the same, e.g., a detectable flow of electrons. For example, the oxidase shown in Table 1 all reduce oxygen to hydrogen peroxide even though they all oxidize quite different substrates. Using the methods described herein, a library of variants of natural oxidases is created that can oxidize a variety of natural and non-natural, i.e., synthetic molecules, such as small molecule drugs. Because the catalytic activity of all such oxidase variants is the same, (oxidation of analyte occurs by reduction of oxygen or a mediator), the readout is also the same (e.g., peroxide or a reduced mediator molecule). The mediator of choice for this process transfers electrons efficiently to the electrode from the enzyme with no interference from other electrochemically active species in the sample fluid. Current commercial sensors suffer from interference from molecules such as ascorbic acid because the redox potential of the mediator required for efficient interaction with the oxidases is similar to that of ascorbate. Optimized oxidases created of the present invention are tailored to efficiently interact with mediators that optimally interact with the electrode without interference from the sample.

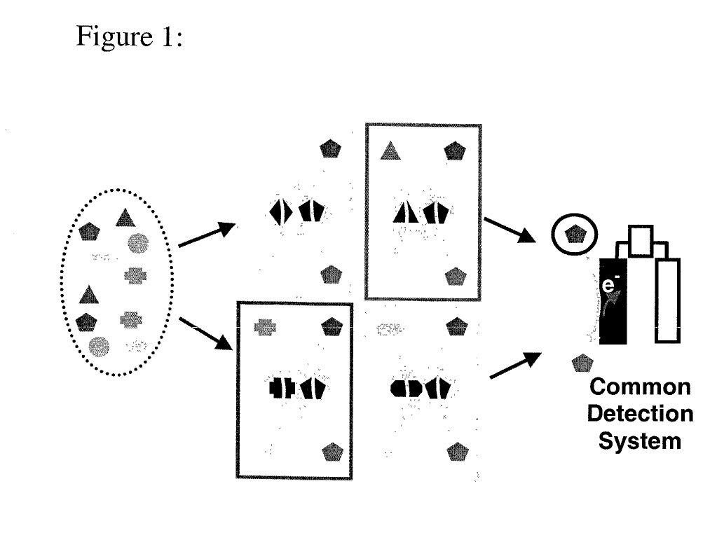

Thus, a common signal transduction platform for all metabolites is produced by arraying a set of oxidase enzymes in a multi-analyte biosensor device that can detect the flow of electrons from hydrogen peroxide to an electrode as illustrated in FIG. 1. Multi-analyte detection systems of the present invention allow metabolites from blood, saliva, urine, sweat, cerebrospinal fluid, tears, or other bodily fluids, and/or from industrial or environmental fluids or gas samples to be measured in real-time, e.g., at a centralized facility, at a point of care (such as a clinic or hopital), or in the home, or field.

It can be clearly seen by one skilled in the art that the common signal transduction platform of the present invention can be readily adapted for use with any set of proteins having a common output, such proteins include, for example, oxidoreductases that can be evolved to oxidise or reduce the same compound or cofactor, fluorescent proteins whose fluorescence can be evolved to be dependent upon the binding of a small molecule or ion, etc.

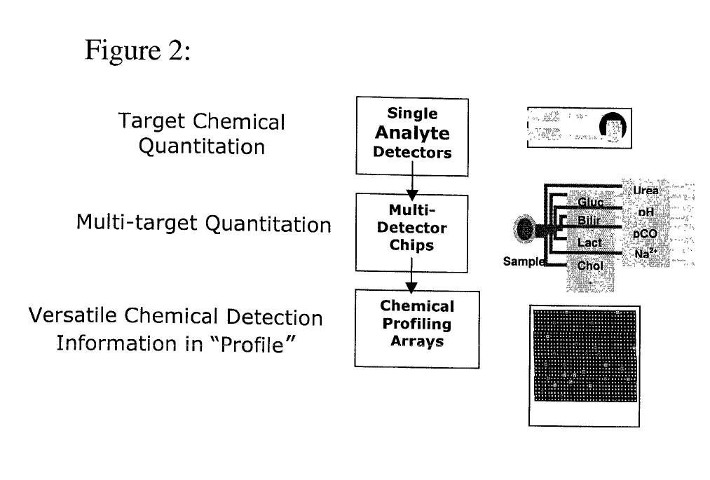

The present invention also provides a multi-analyte detector in which different biopolymer sensing molecules (i.e., biosensors) are created in an array, and the signal produced by each member of the array is measured. The identity and specificity of the biosensor that is generating the signal, e.g., an electrical signal, will be known from its position in the array, which will in turn give the identity and concentration of the metabolite it detects (FIG. 2).

Multi-analyte detectors of the present invention are particularly useful for metabolite measurement. Metabolite measurement (metabolomics) is the least developed of the personalized medicine diagnostic disciplines, measuring compounds that are actually present within the body. For example, genomics can indicate the likelihood of a person developing diabetes; however, measurement of glucose levels in the blood are necessary to diagnose the actual disease state. Multi-analyte metabolomic detection systems of the present invention can be used to identify small molecule markers for other diseases such as atherosclerosis and cancer, and then to assess the progression of those diseases. Hand-held metabolite detection devices of the present invention provide patient-control of small molecule pharmaceuticals; in the same way that diabetes patients can tightly control their glucose levels with a combination of regulated diet, glucose measurement and insulin treatment, a person will be able to control levels of pharmaceuticals by timing doses as a result of accurately knowing their concentrations within his or her body and the concentrations of breakdown or metabolized products that may or may not be toxic. Multi-analyte metabolomic detection systems of the present invention can thus facilitate the use of pharmaceutical agents whose therapeutic indices are unacceptably low for administration without frequent monitoring, i.e., because of a narrow window between efficacy and toxicity.

For example, glucose oxidase is widely used for glucose detection in electrochemical, e.g., amperometric, biosensors. The ability to design the glucose oxidase readout as an electrochemical signal interfaces nicely with existing electronics. This combination of electrochemical signal and electronics is used for quantitation of glucose leading to better dosing regimens of insulin for diabetics and in regulatory circuits for feeding glucose in fermentors.

In addition to amperometric methods, numerous other electrochemical detection systems can be employed in the context of biosensor devices, (including biosensor arrays, as well as single analyte biosensors) such as potentiometric (e.g., pH, selective ion level measurement), and conductive changes (i.e., changes in resistance). Such methods include the use of biosensor biopolymers, especially polypeptides or proteins that upon binding of an anlalyte produce an electorchemally detectable signal, an amperometrically detectable signal, a potentiometrically detectable signal, a signal detectable as a change in pH, a signal based on specific ion levels, a signal based on changes in conductivity, a pizoelectric signal, a change in resonance frequency, a signal detectable as surface accoustic waves, a signal detectable by quartz crystal microbalances, or the like.

Diversification and selection procedures, e.g., shuffling, as described in further detail herein, provide a generalizable approach to producing biosensor devices using electrochemical (and optical, as described hereinbelow) detection methods. In one embodiment, enzymes with electron transport activity, e.g., oxidoreductases or cytochrome P450s, are adapted by directed evolution procedures, e.g., shuffling, to serve as biosensors. Either or both of analyte (substrate) specificity or the ability of the enzyme to function in the context of a sensing device are selectable.

The following table provides an exemplary list of known enzymes that are artificially evolved, e.g., by shuffling and other diversification and selection procedures to detect a variety of medically and environmentally relevant analytes.

TABLE 1
CANDIDATE ENZYMES AND CORRELATED TARGET
ANALYTES

| Enzyme | Analyte |
|---|---|
| Xanthine oxidase | Theophyllin & breakdown products |
| Cytochrome P450s | Warfarin, Cholesterol, Pharmaceutical |
| | Agents |
| Lactate oxidase | Lactate |
| Lysine oxidase | Lysine |
| Galactose oxidase | Galactose |
| Cholesterol oxidase | Cholesterol |
| Alcohol oxidase | Ethanol |
| Pyruvate oxidase | Pyruvate |
| GBlutamate oxidase | Glutamate |
| Choline oxidase | Choline |
| Bilirubin oxidase | Bilirubin |
| Adenosine oxidase | Adenosine |
| 3-P glycerol oxidase | Glycerol |
| Ascorbate oxidase | Vitamin C, OP Pesticides |
| Fructose dehydrogenase | Fructose |
| Methylamine dehydrogenase | Methylamine |
| Nitrate reductase | Nitrate |
| Polyphenol oxidase | Phenol |
| Formaldehyde dehydrogenase | Formaldehyde |
| Fumarate reductase | Fumarate |
| Cellobiose dehydrogenase | cellobiose lactose |

Numerous other suitable enzymes are listed in Table 5.

The present invention proveides a “chip” or handheld biosensor device suitable for home or point of care, e.g., for clinic or hospital use. Production of the chip or device typically involves generation of a set of enzymes or fusion proteins that specifically recognize various analytes (e.g., from a single enzyme or fusion protein in a dedicated single-analyte device, to from 10 to about 50 fusion proteins in a limited analyte device, to about 100, often about 500, frequently about 1000 or even more enzymes or fusion proteins in a multi-analyte device). The set of enzymes or fusion proteins is then immobilized, e.g., onto a substrate or surface of a mixing/incubation chamber on a chip. If desired, the fusion proteins (or other biopolymers according to other applications) can be adapted, e.g., by directed evolution procedures including shuffling, to be compatible with convenient fabrication methods, e.g., screen printing and thin film fabrication. The biosensor proteins can be formulated in a single mixture containing immobilization matrix (e.g., a carbon-based matrix such as carbon ink, a polymer based matrix, which is crosslinked or not crosslinked, ar the like) and all other necessary chemical components and directly printed on an electrode surface. For example, after purification of the biosensor polypeptides, e.g., using a multi-Histidine tag (His-tag), the fusion polypeptide is added to a composite consisting of an immobilization matrix, buffer, necessary electrolytes, and a redox mediator. The mixture can then be directly applied to an electrode surface and dried. A variety of suitable electrode matrices exist, and can be selected by one of skill in the art. For example, a suspension in a conductive carbon ink containing buffer and ferricyanide as a redox mediator can be used. Alternatively the matrix base could be a cross-linked gelatin, a conductive polymer, or a microcrystalline cellulose gel deposited on the surface of a platinum or palladium electrode.

If desired, auxilary components, e.g., cofactors, buffers, or other reagents can be immobilized in a separate detection chamber, for example, allowing for rapid replenishing or replacement. Channels can be oriented connecting the detection and mixing chambers, allowing for instantaneous sample preparation (e.g., by incorporating filters or chromatography materials). In some variations, the bound analyte serves as the substrate for catalytic production of a product. In other variations, analyte binding induces an active conformation of a catalytic site that acts on a secondary substrate. In the latter case, the substrate, (chosen to be appropriate for the enzyme variant used in the biosensor fusion) can be supplied in immobilized form in the detection chamber, or added to the mixing/incubation or detection chambers as required. In the event that members of the multi-analyte array require different substrates for signal production, it is most convenient to immobilize each substrate in an assigned position, permitting deconvolution of the signal to yield specific information regarding the bound analyte.

To perform an assay, the chip is placed in, e.g., a handheld device equipped with elecrodes positioned to interface with the detection chamber. Sample is added to the mixing/incubation chamber, and the sample is incubated with the biosensor to permit formation of a signal, e.g., conversion of a substrate to a detectable product, oxidation or reduction of a mediator, optical changes, etc. The product is then transferred, e.g., under pressure, through a regulatable gate or membrane, or by diffusion, capillary action, capillary electrophorsis, centrifugation, etc., into the detection chamber. If desired, the detection chamber is washed with buffer, e.g., from the mixing/incubation chamber or from a separate wash buffer entry. If necessary, additional detection reagents are also added to the detection chamber, and the result of analyte binding is provided as a readout of the hand-held device, e.g., on an LCD.

Such a system provides the means for analyzing a large variety of very different analytes on a single platform at the same time in the form of a digital readout. The advantage of this system is that a lot of very different analytes can be measured in one platform at the same time in the form of a digital readout. For example, in one embodiment of an oxidase-based biosensor of the present inveniton, variants are employed that can oxidize a variety of natural and non-natural, i.e., synthetic molecules, such as small molecule drugs. Because the catalytic activity of all such oxidase variants is the same, (oxidation of analyte occurs by reduction of oxygen or a mediator), the readout is also the same (e.g., peroxide or a reduced mediator molecule).

To insure optimal performance of the multi-analyte array, the dynamic range of the biopolymer-analyte recognition event is selected to correspond to the range of analyte concentrations found in the biological sample. In applications where protein analytes are to be assessed, the Kd of each analyte-specific binding domain is adjusted to the range of analyte concentrations found in the sample. This can be accomplished by selecting enzyme variants with higher catalytic activity for analytes that are present in lower concentrations, and variants with lower catalytic activity for analytes present in higher concentrations, or by evolving a set of more than one enzyme using, for example, the methods described herein, so that the dynamic range of the set of enzymes includes all concentration of analyte that will be encountered in the samples to be analyzed.

Signal Transduction

A variety of signal transduction mechanisms are suitable for use with the methods and devices of the present invention. For example, signal transduction mechanisms in biosensor devices of the present invention may be are electrical or electrochemical in nature. For example, an oxidoreductase enzyme, such as glucose oxidase, will catalyze a flow of electrons from a target analyte to an electrode, directly or via a mediator, which is easily detected as an increase in electrical current.

Biopolymers of the present invention, such as enzymes, can also be used to indirectly measure substances that cannot easily be oxidized, such as iron, phosphate, calcium, etc. This can be done by evolving an enzyme that oxidizes a common abundant metabolite such as glucose or urea, and making a variant or a set of variants that respond differently to (i.e., are inhibited or activated by) the presence of the desired analyte (e.g., iron, phosphate, calcium, etc.). Analyte concentrations can then be calculated by comparing activities of the set of enzymes.

Signal transduction may be facilitated by the use of conductive polymers, such as, e.g., polyaniline as the matrix for protein binding, which facilitates electron transport to the solid electrode surface. The proteins are directly wired to the conductive polymer, which forms the electrode. The polymer is connected with the solid state electronics that transfer the signal to the detector.

The most direct method to measure the activity of an electrochemically active biopolymer (e.g., protein) is to place the biopolymer on an electrode and to measure its response to a stimulus. Biopolymers employed in biosensors of the present invention are tailored to resist loss of enzyme activity (e.g., via denaturation at the electrode surface or intolerance of the immobilization method), poor electron transfer to the electrode, altered substrate specificity, and poor reproducibility, thus providing for simplified sensor construction.

The present inventio also contemplates the use of naturally occurring and modified electron transport proteins to facilitate signal transduction. In nature, a variety of electrochemically active proteins are part of an electrochemical gradient in which the energy liberated, e.g., from light or food, is used to drive work until the electrons are delivered to the final electron sink, i.e., oxygen. The sole function of these proteins (see e.g., www.chem.qmw.ac.uk/iubmb/etp/) is to take electrons at one redox potential and pass them on to another protein in a controlled way. An example of this is found in cytochrome P450 chemistry, which is described further in Example 5. In one example, the electrons originate in NADH where they reduce feredoxin reductase, which reduces feredoxin, which passes the electrons to the P450 itself. This cascade enables the biological system to control the electron transfer and prevent the electrons flowing at will (equivalent to shorting a battery).

These electron transport proteins tend to be small stable proteins, which modulate the activity of the proteins on either side of the chain by binding events and of course by electron transport. In the biosensors of the present invention, these proteins are used as molecular wires between the electrode surface and the sensing enzyme of interest. Each new protein of interest is fused to a suitable electron transport partner (either its native partner or with an artificial partner with matched redox potential). A series of electron transport proteins is, therefore, produced that act across the relevant redox levels, and that are both stable on a selected surface and readily accept electrons from the selected surface. If desired, the proteins are modified and selected such that their tertiary structure forms a surface that binds to the surface of the electrode in a consistent orientation.

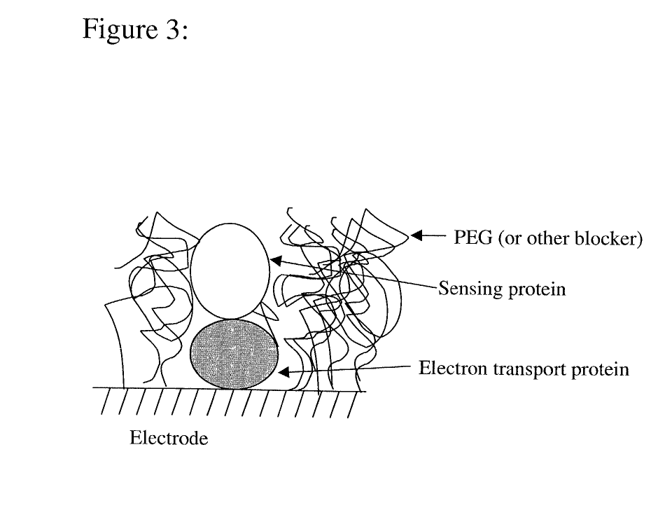

As illustrated in FIG. 3, the electron donor and electron acceptor proteins can be fused, and the complex can be further diversified using methods described herein, and selected to optimize electron transfer between the two proteins.

One advantage of this approach is that only a small subset of proteins need be optimized for surface stability and metal-protein electron transfer, facilitating development of a generalizable biosensor platform. In a manner analogous to antigen binding affinity in the immune system, it is only necessary to alter the analyte binding moiety to correspond to the analyte(s) of interest. Additional details regarding directed evolution of monooxygenases can be found, e.g., in WO 00/09682, published Feb. 24, 2000.

Incorporation of a multitude of enzymes that are all optimized and standardized for function on a single platform permits the production of a detector that is able to analyze multiple metabolites simultaneously from one biological fluid sample.

Such portable, easy to operate devices will diminish the need to perform analyses in central labs or by highly qualified, specialized personnel. The amount of data that can be gathered by a broader user population will be very large, creating an information database that can be applied to improving the quality of diagnosis and treatment, as well as to administering diagnostic and treatment protocols. In one application, biosensor devices of the present invention permit metabolite fingerprinting by an array of enzymes or other biosensor biopolymers providing a powerful tool to monitor complex disease states or progression of disease states. The biopolymers or enzymes on the array can be optimized variants to function on the electrode surface or a library of, e.g., shuffled, variants that show diffeent substrate specificities. After initial training of array data on a representative number of individual phenotypes, diagnosis is performed by wireless transfer of newly acquired data to the database and by correlating to existing information. Each new data acquisition will also contribute to the diagnostic value of the database.

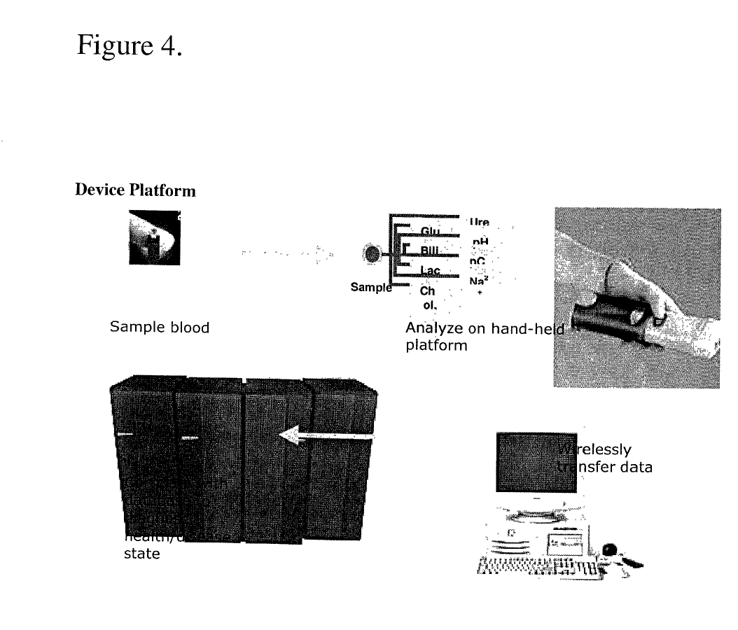

An exemplary device platform, e.g., for medical diagnostic or monitoring applications, is illustrated in FIG. 4. The platform includes, (a) a fluid sampler (e.g., for obtaining blood, urine, sweat, tears, cerebrospinal fluid, etc.); (b) a fluid test strip containing fluid-flow director and biosensor, or biosensor array, coupled to signal transduction mechanism; (c) a hand-held reader for measuring signals from biosensors; (d) a mechanism for wireless transmission of data to a receiver (e.g., either in the home or on a remote server, e.g., at a point of care such as a clinic, hospital or other service provider). Data is then transmitted from the biosensor device to a data collection and processing unit, e.g., in the home or on a server at a point of care. Data relating to analyte binding to the biosensor device is processed and transmitted back to the device, where it is interpreted and read-out for the user. In an implantable version of a biosensor device, the fluid sampler, test strip and hand-held reader are replaced by a single implantable sensor.

Instructions can relate to disease state, e.g., diagnosis or prognosis, or to management issues, such as regulation of dosage of a drug or drugs. The read-out can take the form of quantitative or qualitative values, to be interpreted by the user or a care provider, or can be directives, e.g., “It is time for your next dosage,” “You are at high risk for a heart attack, call 911,” and the like. Alternatively, and especially in an implantable device, e.g., coupled to an administration device such as an insulin pump, the processed data can be used to directly control treatment. For example, to reduce toxicity or to insure compliance with a protocol, a pill-dispenser could be controlled by the device, such that medication, e.g., pills, are only dispensed in response to information gathered and transmitted by the device.

Biosensors of the present invention can be used in connection with other devices, e.g., using a MicroElectroMechanical Systems (MEMS) based approach. For example, implantable biosensors can be connected to a pump, and the signal created by the sensor can be transmitted to the pump to deliver the medication instantaneously and in an appropriate dosage. A classic example is the artificial pancreas with a sensing unit (glucose oxidase) connected to an insulin pump. Implantable devices have to meet even more stringent criteria than single timepoint sensors. Due to the necessity of minor surgery the implantable sensor should last as long as possible without intervention which requires higher stability of the sensing biomolecule than most natural enzymes are designed for. Optimization of biomolecules that recognize biomarkers in vivo for disease diagnosis and treatment can be achieved by directed evolution, e.g., by shuffling.

Production of Novel Sensing Molecules and Arrays

Shuffling and other diversity generation reactions can be used either to optimize the binding/reaction of a single protein with its substrate, or to create a family of related proteins with different substrate preferences. These proteins can be used individually or as arrays on a solid support, for example an array on a glass slide or chip, a microtiter tray, or the like. The read-out from a biosensor can be either a single quantitative measurement, or a pattern of signals from the array. Individual signals can be quantitative or qualitative (i.e., yes/no indications, or more subtle intensity measurements), with overall patterns including any of: positive, negative, or partial signals. The arrays can be used to detect the binding or interaction between an array and one or many different molecules or other stimuli.

An alternative to the solid phase array is pooled bead biosensors, which can be in an array format, or can exist as individual array members. For example, dotting His-tagged protein could be achieved directly from a library by automated HTP protein purification followed by arraying the purified protein on a Nickel-NTA coated surface. Other binding motif/receptor partners would work analogously. The surface could be the beads used in the purification if the beads are individually addressed. Different size beads with different fluorophores (eg. quantum dots) can be distinguished by fluorescent correlation spectroscopy, or other methods. Also, combinatorial-chemistry sample tracking methods (e.g., GC tags, etc.) are applicable.

Each library clone is optionally given a defined bead or spatial address/marker and permanently bound, e.g., in bulk. In bead embodiments, the beads are pooled and used as a mixture. On exposure to a signal molecule, a particular bead that lights up is identified and decoded back to an original clone. For sensor applications, the pattern of clones or other bound elements that show a signal response corresponds to a specific molecule/stimulus (the intensity of the response should quantify the stimulus). For other applications, this technique can be used for catalyst screening when reuse of the same library (e.g. an active lipase library) many times for different purposes is desired.

Enzyme Activity Dependent on Molecule of Interest

In one aspect, the invention provides for diversification and selection, e.g., shuffling, for substrate specificity, enzymatic activity in response to an allosteric effector, such as a metal, a cell surface antigen, or some natural or synthetic small molecule. Selection can be for variants that respond to the molecule as a specific positive effector or an inhibitor of the enzyme. Presence of the analyte is then detected by activity of the enzyme.

Several enzymes require bound metals for stability and/or catalytic function. These sites are often highly specific for a particular ion, depending on the size of the metal binding pocket and ligand geometry and types. There are several reports of altering the metal dependence of enzymes by engineering (for review see Regan, L., TIBS, 1995, 20:280-285 and Shao & Arnold, Curr Opin Struct Biol, 1996, 6:513-518). For example, Haflon and Craik have engineered a trypsin mutant that is sensitive to submicromolar Cu2+ (J Am Chem Soc, 1996, 118:1227-1228). It should be possible to alter specificity of existing metal sites or create new ones by shuffling. In this way an enzyme can be made to be a sensor for a metal of choice.

Subtilisins require bound Ca²⁺ for proper folding. Previous work has shown that there is considerable variability in this requirement among different subtilisins. Variation is seen in the number and affinity of required Ca2+ sites. Engineering and directed evolution have been used previously to alter the affinity of Ca2+ binding and in one work subtilisin BPN was evolved to be stable in the absence of Ca2+. Shuffling could be used to create subtilisins that specifically require heavy metal ions of choice for activity. The presence of the metal ion in a sample could then be detected as protease activity using one of several existing sensitive and rapid colorimetric and fluorimetric protease assays.

Doi et al. (Doi et al.,FEBS Lett., 453,1999) have demonstrated that insertion of a protein domain containing a desired molecular binding site into a surface loop of GFP led to ligand binding with the fluorescent property of the protein. A similar concept can be applied to obtain enzyme-linked biosensors by shuffling proteins with inherent fluorescent property and screening for change of fluorescent properties as a result of binding of the analyte.

Similarly, a protein can be evolved so that it gives a desired signal (such as fluorescence) upon binding to an analyte. Shuffling and other diversity generation/selection methods can also be used to build multiplex detectors, including multiple multi-wavelength protein fluors, and the like.

Creation of Functionally Diverse Arrays

Antibodies can be diversified, e.g., shuffled, to create functional (binding) diversity. It is possible to include differential selection (e.g., to select antibodies that bind to proteins or other compounds present in a diseased sample (blood, CSF, tumour sample, etc.) toxin, biological warfare agent, or the like). Antibodies can be arrayed (optionally including control antibodies for array for normalization), and the arrays can be screened for disease markers in patient samples, environmental samples, biological warfare agents, and the like. For additional details regarding antibody diversification, see, e.g., WO 01/32712, published May 10, 2001. This same strategy can be applied to any other small molecule binding protein family, for example, lipocalins (see, e.g. Beste G. et al., 1999, Proc Natl Acad Sci USA, 96(5):1898-903).

Similarly, olfactory receptors can also be diversified, e.g., shuffled, to create functional (binding) diversity. Optionally, differential selection as used, e.g. to select receptors that bind to compounds present in positive sample (environmental agents, fragrances, metabolites, etc.). Olfactory receptors or binding domains derived therefrom, can be arrayed (optionally including control receptors in the array for normalization purposes). An array can be used, e.g., as an electronic “nose,” to screen for chemeical warfare agents, fragrances, metabolites, etc. in samples.

Light sensitive proteins can also be arrayed. For example, light sensitive bacteriorhodopsin, eye photoreceptor proteins (e.g., from rods/cones etc) can be evolved to change the responsive wavelength range (e.g., out to the UV/IR spectrum) for the protein. These proteins can be arrayed and molecular cameras, film, and the like can be made from arrays of these proteins.

Enzymes can be diversified into families with different substrate specificities. Enzymes can also be diversified into families with the same substrate specificity but different sensitivities to analytes which may bind to the enzyme and affect their activity competitively, non-competitively or allosterically. Arrays of such enzymes can thus be sued to measure the concentrations of multiple analytes of similar or dissimilar structures simultaneously.

Optimization of Physical Properties

Many array specific activities and activities related to function of a biopolymer as a biosensor, e.g., in a sensing device, can be selected for, including shuffling for stability in an array, e.g., in a specific array or device format. Similarly, sensor arrays can be selected, e.g., following diversification by shuffling or other procedures, to decrease array costs and to increase array storability. Thus, shuffling or other diversity generation/selection schemes can be used to increase stability of a biosensor biopolymer or array, stability to the arraying process(es), stability of the array to long term storage both in manufacturing and in any sensor device where the conditions will vary (e.g., at least one year stability with little no activity drop-off and some internal calibration can be produced), stability to the conditions that the sensor are used in (e.g., biological fluids for medical use, particular climates such as desert or cold climates for military use, zero gravity for space, pressure sensitivity or insensitivity), and the like.

It is also possible to select for orientation of array components on the arrays. For example, a protein sensor is reproducible if all the proteins in an array are held in the same orientation relative to a surface of the array. This can be accomplished, e.g., by attaching a binding tag/surface at the same point in each protein followed by shuffling for optimal activity. This creates regular 2D arrays of proteins, which are readily visualized/studied by AFM/Neutron scattering/X-rays, etc. Also, surface properties of protein coat can be made uniform (e.g., with respect to friction, hydrophilic/hydrophobic/aspects, etc.). Ridges of materials can form diffraction patterns, which can be modified by perturbations of the protein surface, e.g., as brought about by binding of materials to the protein, or by heat, light, or the like. Similarly, the array can provide an optical equivalent to surface plasmon resonance. This can also be achieved with membrane proteins on a tethered membrane surface.

In general, proteins are expensive to produce. To reduce array costs, the array components (e.g., proteins) can be selected during shuffling or other diversity generation/selection methods for easy production and purification. Over-expression mutants can be a goal of shuffling or other diversity generation methods. For example, shuffling can be used to provide a super folded mutant, increasing the yield of functional protein in a preparation. Similarly, fermentor time is expensive; thus, shuffling for fast/early expression, or permanent enzyme secretion in a filtration tank production fermentor for protein production can be performed. With respect to expression, it is sometimes desirable to select for super high potency operators/ribosome binding sites to increase the percentage of total carbon/nitrogen going to a protein of interest.

One aspect provides for selecting, e.g., following diversification, e.g., by shuffling, for avidity and/or selectivity. For example, an extremely diverse library can be made and screened for binding to a specific chemical (e.g., in a bead assay format) in the presence of high concentrations of other components and different chemical displaying beads. The bead of interest, comprising diversified, e.g., shuffled components of interest can be isolated to find out what bound to the bead. The library can be re-challenged with new bead bound chemicals to get new binders.

The detection limit/range of an enzyme (e.g., as a sensor) is partially dependent upon its Km for the substratelbinder of interest. Thus, the Km can be selected for to the value of interest for an intended sensor application. Of course, the relevant Km will vary, depending on the system of interest—for example, different sensitivities are appropriate for, e.g. a glucose sensor in blood as compared to glucose in fermentor.

Signal Transduction

Responses can trigger a cascade to increase sensitivity of a given assay. For example, a downstream cascade can be created or optimized by selecting the desires activities following diversification, e.g., by shuffling, of a library.

Similarly, the catalytic mechanism of the sensor protein can cause the production of a measurable side product (e.g., H₂O₂ by oxidases, for example, glucose oxidase). The enzyme is selected to be specific to other substrates, to have a Km in the desired range for the sensor application, to have a desired stability, to avoid the need for expensive/unstable cosubstrates/cofactors, etc.

Allosteric Biosensors

A common method for monitoring enzyme reactions is to use an analytical assay for specific or generic detection of products, e.g., by mass spectroscopy or by exploiting fluorescent or chromogenic properties of the compounds produced. Mass spectrometry is highly specific and sensitive, as well as broadly applicable, but is not amenable to ultra-high throughput. Chromogenic and fluorescent assays are readily adapted in scale and efficiency for high throughput applications; however, most enzyme products are not chromogenic or fluorescent, thus limiting the scope of metabolites that can be monitored.

The present invention provides ways of producing and identifying bifunctional enzymes that can be used as sensitive sensors for enzyme products, e.g., small organic molecules or ionic species. In some cases, binding of an analyte of interest at an allosteric site would be coupled to signal-transduction function, e.g., by inducing the proper active conformation of the evolved enzyme and monitoring the enzyme activity using simple optical methods like fluorescence or colorimetry. In other cases, enzymes are produced that are already fluorescent, and an increase or decrease in fluorescence is induced upon binding of an analyte. The following are illustrative examples of potential bifunctional enzyme based sensors.

Interdomain Cross-Talk:

In the case of Nitric oxide synthase (and other cytochrome P450s and some flavo-proteins) the binding of substrate to the active site causes a change in shape and reduction potential that allows the transfer of electrons from NAD(P)H reductase. This can be observed by NADPH depletion (e.g., by absorbance at 340 nm). The binding site of this system can be selected to be specific for the molecule the sensor is designed to detect.

For example, the heme-binding pocket is extremely widely used in nature to effect signaling and catalytic functions for many molecules. For example, the binding pocket is relevant to gases (O₂, CO, NO), ions (N₃, CN), small molecules (steroids, polyketides, aromatics (xenobiotic metabolism in animals and microbes), terpenes, fatty acids, amino acids. Some hemoproteins, such as cytochrome b5, primarily bind and transfer electrons. Nitric oxide synthase includes heme and calmodulin-reductase domains. In this system, electron transport and catalysis relies on calcium bound to calmodulin.

NADPH is expensive/unstable and thus not ideal for many sensor applications. A better signal generation approach is a direct measure of the change in reduction potential. Solid state electrochemical detectors perform this task and, because they can be microfabricated, are well suited to microarray technology. In this example, an array of individually addressed electrochemical detectors is created in a silicon chip (densities of ˜10 000 per square centimeter can readily be achieved). The surface of the silicon chip can be treated to provide an environment amenable to protein attachment and stability (e.g., lipids on a surface, PEGylated surfaces, specific charge environments, chemical functionalities such as Ni-NTA, etc.). The coating is designed not to interfere with the sensor (e.g., is not electrochemically active, etc.).

The sensor proteins are arranged or arrayed on top of the sensors. Binding of molecules to the heme pocket of the sensor protein changes the reduction potential of the protein and results in an electrical signal. Each binding event gives a signal leading to a quantitative response to binding. The sensor proteins are characterized before attachment or the pattern of response for each stimulus could be trained into the sensor. In order to stabilize the sensor, the surface coating is optionally polymerized to permanently attach the proteins and associated molecules to the surface.

Inter-subunit crosstalk (allosteric responses) can also be detected. For example, proteins change shape upon binding of their target. This movement can transduce energy across the molecule to cause secondary effects. Oxygen binding by hemoglobin is a classical example of this. Hemoglobin type structures can be used, e.g., where one subunit is sensitive to a molecule of interest, changes shape on binding and causes a shape change in other subunits, leading to a measurable change (catalysis etc.).

Diverse or specific binding domains can be generated. For example, an allosteric protein can be shuffled such that binding a target molecule initiates an allosteric change in other subunits of the molecule. The other subunits respond with a detectable change in binding or catalytic activity. For example, oxygen binding to hemoglobin changes the protein's absorption maximum, which can be read by a laser.

Transcriptional regulators can also be adapted, e.g., shuffled, and utilized as biosensors. For example, a cell-based sensor in which cells contain different transcription factors sensitive to binding events (e.g., a lac repressor, a regulators of aromatic catabolism, etc.) can be made. The presence of an activator produces a signal, e.g., transcription of GFP, luciferase, beta-galactosidase etc. Cell-based biosensors can be made, e.g., by making multiple related cells (e.g. by whole genome shuffling as noted in the references above) and detecting small molecules, e.g., by a respiration pattern of a microbe array.

Direct detection of small molecule binding to transcriptional regulator using an array of target DNAs (or RNAs) can also be performed. For example, a regulator is optionally physically bound to a target sequence, thereby measuring presence of activator molecules in sample. Direct physical detection of binding can be performed by measuring a change in the reduction potential. For example, cytochrome P450s change reduction potential on binding to a substrate. Measurement of this change is, thus, electrical, which is a preferred readout mechanism/effector. Surface plasmon resonance can also be used, e.g., to detect protein-protein interactions such as antibody-antigen binding. Other approaches include the monitoring of fluorophores on bacterial spores activated by binding of spore to target molecule.

In one aspect, an orientation change is measured. For example, if the proteins of a sensor array have been deposited to give a specific optical diffraction then binding events will perturb the signal. Surface plasmon resonance also responds to binding in this way. Each sensor protein (or closely grouped identical members of an array) acts as a pixel, which changes individually, based on binding to a specific agent. Small pixels are picked up by a CCD camera for example. A larger pixel can be visually observed in the protein equivalent of a LCD device.

The array can be printed onto a clear sheet and arranged so the surface becomes opaque on ligand binding. This provides a robust cheap sensor, suitable for industrial or military uses. For example, a helmet visor can be constructed using this technology to automatically respond to the presence of environmental agents, contaminants, toxins, chemical warfare agents, or the like, providing an immediate displayed response by the array.

Optical change provides one preferred approach to array monitoring. For example, a protein can be shuffled such that the sensor protein carries a fluorophore (e.g., GFP) which is quenched under normal circumstances (e.g., tryptophan can act as a quenching agent in the correct orientation/proximity). On binding of a target molecule to the array, the array protein members change shape and move the quenching agent away form the fluorophore, giving a measurable increase in quantum yield/emission wavelength. Other markers include FAD fluorescence or a fluorophore (e.g., FITC etc.) that is chemically conjugated to a specific lysine or cysteine, etc., which are also quenched until target molecule binding occurs.

Identification of Enzmes that can be used as Heavy Metal Detectors.

A number of known enzymes require bound metal ions for stability and/or catalytic function. The ion binding sites of these enzymes are often highly specific for a particular ion, and binding depends on the size of the metal binding pocket and ligand geometry and charge. There are several reports of altered metal dependence by protein engineering (for review, see, e.g., Regan, L., TIBS 1995, 20:280-285 and Shao, Z. & Arnold F. H., Curr Opin Struct Biol, 1996, 6:513-518). For example, Haflon and Craik have engineered a trypsin mutant that is sensitive to submicromolar Cu²⁺ (J Am Chem Soc, 1996, 118:1227-1228). In the methods of the invention, library arrays are produced that include members with altered specificity of existing metal sites or novel metal binding sites. These library arrays, or alternatively, selected library members, can be used as sensors for one or more metal ion of choice.

For example, subtilisins require bound Ca²⁺ for proper folding. Previous work has shown that there is considerable variability in this requirement among different subtilisins. Variation is seen in the position and affinity of required Ca²⁺ sites. Engineering and directed evolution have been used previously to alter the affinity of Ca²⁺ binding (Pantoliano, M. W., et al, Biochemistry, 1988, 27:8311-8317). In one work subtilisin BPN was evolved to be active and stable in the absence of Ca²⁺ (Strausberg, et al, Biotechnology, 13:669-673). DNA shuffling or other diversity generating methods can be used to produce a diverse library of subtilisins or any other enzyme class, wherein individual members specifically require various heavy metal ions or other analytes for activity. In essence, this produces an array of bifunctional heavy metal binding/protease enzymes. The presence of one or more metal ions in a sample is detected based on protease activity of the array of subtilisin variants using one of several existing sensitive and rapid protease assays. Similarly any enzyme or family of enzymes may be made dependent upon or may be made to be inhibited by any metal, ion or other small molecule. Comparison of the activity of an enzyme sensitive to the concentration of an analyte with a reference protein that is not sensitive or is differently sensitive to that analyte, will allow the concentration of the analyte to be determined.

Evolved Enzymes that can be used as Anionic Leaving Group Detectors.

Enzymatic reactions involving nucleophilic substitutions result in small organic or inorganic anionic leaving groups (e.g., chloride, fluoride, bromide, iodide, sulfate, phosphate, phenolate, carboxylate, etc.). A selective and sensitive method for quantitative measurement of these generic leaving groups is desirable for assays that can be applied to a variety of different enzyme classes. Doi et al. (Doi et al.,1999) have demonstrated that by inserting a protein domain containing a desired molecular binding site into a surface loop of GFP, they could couple ligand binding with the fluorescent property of the protein. A similar concept can be applied to obtain enzyme-linked biosensors by arraying libraries of bifunctional GFP-like proteins and screening for change of fluorescent properties of the protein upon binding of the analyte of interest.

Evolved Enzymes that can be used as Small Molecule Sensor.

Libraries of bifunctional reporter enzymes based on GFP-like proteins that indicate presence of a small molecule in a sample are also a feature of the invention. For example, binding of the specified small molecule, or group of molecules, is detected as induction or alteration in fluoresecence of the bifunctional GFP/binding protein.

Biosensors based on Calmodulin Variants

One particularly valuable attribute of many proteins is that they can change in conformation upon binding to a specific molecule, or analyte of interest, even in the presence of a wide variety of structurally similar or unrelated molecules. Even in a complex medium such as a cellular extract, or biological fluid such as blood, proteins specifically bind to a particular analyte in solution in a concentration dependent manner.

In the context of the present invention, proteins that change conformation (e.g., either allosterically in a fusion polypeptide or as an single polypeptide) in the presence of relevant (e.g., physiologic) concentrations of the analyte of interest. As described herein, the protein can be derived by directed evolution, e.g., shuffling, to alter specificity or affinity to detect the analyte of interest under specified conditions, thus, producing a protein (or collection of proteins) that binds to the analyte(s) and undergo a conformational shift which is detectable by the sensing element.

For example, the protein calmodulin exists in an extended conformation in the absence of Ca⁺⁺ (or under physiologically low levels of Ca⁺⁺). As calcium levels increase, the molecule curls around the Ca⁺⁺ ion to form a V shaped molecule. This brings the two ends of the protein into close proximity. In situ, this conformational change results in induction of downstream events. If desired, the selectivity of calmodulin for calcium can be modulated, e.g., by shuffling or other directed evolution procedures, for example, for selectivity for other divalent metal ions, other ions of interest, or other molecules of interest (e.g., by such methods as phage display).

In the context of a biosensor, the two ends of the molecule can be two individually inactive domains of a protein that when in brought into proximity by a conformation shift become active. Tyrosine Kinases are often activated in this manner in the signaling cascade. A candidate protein, such as alkaline phosphatase or horse radish peroxidase (or any protein that generates a detectable signal, e.g., colorimetric/fluorogenic or electrochemical signal) is constructed as a fusion protein such that the signal generating (e.g., catalytic) site is separated by the calmodulin domain into two separate domains (the two domains can be chosen randomly or on the basis of structural criteria, e.g., X-ray structures, etc). Variants are then selected that are active only in the presence of calcium.

Alternatively, the two domains separated by a calmodulin domain can be an electron transport protein (e.g., from a cofactor molecule) and, e.g., the catalytic unit of endothelial Nitric Oxide Synthase (eNOS). It will be appreciated that the conformational shift which activates production of a signal, can be either primary (i.e., induced directly by binding of the analyte) or secondary (i.e., due to displacement of an inhibitor by the analyte which induces a conformational change that activates the signal production domain(s). The latter are, generally considered, allosteric conformational changes, where binding of an analyte to a binding domain induces a conformational change that places the catalytic domain of the polypeptide or protein in the correct structural orientation to bind substrate, and catalyze the conversion of substrate to a detectable product. The analyte, or compound for which the binding domain of the biosensor is specific is typically unrelated the substrate catalyzed to generate a detectable product.

In one embodiment, the two signal generating domains separated by calmodulin contain spectrally matched dyes (e.g., GFP/RFP, Fluorescein/rhodamine, europium cryptate/APC etc.) enabling detection of the conformational change by FRET (or time resolved FRET). For example, using rational design strategies, GFP calmodulin fusions that are responsive to calcium have been produced (Baird et al. (1999) Proc. Natl. Acad. Sci. USA 28;96:11241-6; Topell et al. (1999) FEBS Letters 457:283-289). The methods, including a variety of diversification and selection procedures, including shuffling, can be used to produce binding domains that are specific for a wide variety of analytes, especially small molecule analytes, other than calcium ions.

In addition to calmodulin, numerous other proteins are known that undergo analogous conformational shifts. For example, two inactive catalytic domains of a G-protein coupled receptor become active when the sensing domain binds the target analyte and, e.g., dimerizes into a complex. Similarly, nuclear hormone receptors undergo a conformational shift that causes them to traverse the nuclear membrane and bind to specific DNA sequences upon binding to their ligand. Nucleic acids encoding such proteins can serve as the starting materials for the directed evolution of proteins, with specificity for an analyte of interest. Signal generation is based on optical detection of the conformational change as described herein.

For example, fusion proteins based on a bipartite green fluorescent protein (GFP) can be produced which have any of a variety of analyte binding domains, calmodulin, G-protein coupled receptors, nuclear receptors, olfactory receptor, lipocalins and antibodies being only a few of the examples. Directed evolution procedures, e.g., shuffling, can be used to derive binding site variants that are specific for an analyte of interest. In particular, such fusion proteins can be used to produce biosensors specific for a wide range of non-nucleic acid analytes, in particular small molecule and protein analytes. Similarly, procedures such as shuffling can be used to generate and select GFP fusion variants that exhibit the desired conformational changes, i.e., from inactive in the absence of analyte to fluroescent upon binding of the specified analyte.

Alternatively, a change in conformation can allow an electrochemically active group to contact (electrically, either directly or through intermediates) an electrode. In this case binding is measured by a change in current or potentiometrically, etc. The change in conformation can also be observed by binding to a specific surface by surface plasmon resonance (SPR) microbalances, or the like.

Optical Detection Methods

A variety of optical detection methods are employed in the context of the biosensors, sensor arrays and devices of the present invention, e.g., by ultraviolet spectrophotometry, visible light spectrophotometry, surface plasmon resonance, fluorescence polarization, fluorescent wavelength shift, fluroescence quenching, colorimetric quenching, fluorescence resonance energy transfer (FRET), liquid crystal displays (LCD), and the like. Numerous such methods are known in the art, and well described in the patent and technical literature.

In brief, surface plasmon resonance can be used to detect alterations in the diffraction of light due to binding of an analyte. For example, surface plasmon resonance detects a change in the angle at which light hits a detector between a substrate bound biopolymer that has bound an analyte molecule and a biopolymer that is unbound to analyte.

In fluorescence polariztion, a fluorescently labeled analyte is bound to a biopolymer. When analyte is added, e.g., in a sample, it displaces the labeled analyte from the protein. The liberated fluorophore has a significantly lower polarization than the bound fluorophore, resulting in a detectable change in the signal. Similarly, a fluroescent wavelength shift is based on the finding that many fluorophores exhibit a change in excitation and/or emission wavelengths and quantumyield of fluorescence (E) when released from, e.g., a hydrophobic binding site on a biopolymer, to an aqueous environment.

Fluorescence quenching also involves a change in fluorescence that is dependent on binding of an analyte to a biopolymer. In this case, however, the fluorophore is designed to be quenched, e.g., by the indole ring of tryptophan. To employ fluorescent quenching, the biopolymer, or members of a biopolymer array, are selected or engineered to incorporate a tryptophan optimally positioned to quench excitation of the bound fluorphore (i.e., in an orientation and proximity for pi cloud overlap). In one variation, fluorescent quenching involves the use of a fluroescently labeled analyte analogue. Upon binding of the analyte of interest, the labeled analogue is displaced and the quenching is removed. Alternatively, a fluorescent dye unrelated to the analyte is placed in proximity to the sensor biopolymer, e.g., by thethering as described below. Binding of an analyte induces a conformational change in the biopolymer that moves the dye relative to the tryptophan, releasing the dye from quenching.

In another variation of a fluorescent signal dependent on a conformational change in the biopolymer, two domains of a fluorescent protein, e.g., GFP, are separated by an analyte binding domain in the context of a fusion protein. Upon binding of the analyte, the two domains required for fluorescence are brought into proximity to allow detection of fluorescence, e.g., by FRET.

Another approach involves the use of calorimetric quenching. As described above a dye molecule is bound to the biopolymer. In this case, however, the dye is not fluorescent or optically quenched, rather the dye is an unstable molecule (such as the X of X-gal, or a standard indole), that is initially bound to the sensor biopolymer. When bound to the biopolymer the dye is unreactive, however, upon analyte binding, the dye is displaced and becomes reactive, e.g., to oxidative dimerization, resulting in the formation of an insoluble colored precipitate. This method is particularly suited to certain medical, diagnostic and monitoring applications as the biosensor can be prepared in a “dipstick,” tape or ribbon, capsule, or other easily stored and manipulated format.

An LCD involves a two dimensional liquid crystal of the sensor biopolymer, arranged in a single orientation on a substrate. Exposure to the analyte induced a conformation change in the biopolymer resulting in a deformation in the crystalline packing. This, in turn, makes it easier for the surrounding elements of the liquid crystal to bind and change conformation. This cooperative cascade alters the optical properties of the display, and amplifies the signal generated by analyte binding. In particular, use of such a detection method is preferred in applications (e.g., environmental monitoring), in which it is desirable that a certain analyte concentration be required to initiate the conformational shift, but once initiated, the signal develops rapidly and with increased amplitude.

In addition to the detection methods described above, a variety of light activated biopolymers can be used in the context of the biosensor devices of the invention, e.g., photoactivatable enzymes including photoactivatable nucleases or polymerases for sequencing, photoactivatable enzymes for complex combinatorial biosyntheses, e.g. using photographic masks and the like. For example, in the context of a biosensor array, one approach is to bind a first substrate to a support, add a second substrate and transiently photoactivatable enzyme, then photoactivate the array or array components using a mask or laser scanning method to activate only a desired subset of the enzyme. The enzyme is removed and a second substrate added, followed by addition of a third substrate and second transiently photoactivatable enzyme. This process is repeated using a second mask or laser scanning pattern. This is, essentially, a solid-support based combinatorial chemistry method. An array/library can be assayed, for example, for cellular effects (such as antiibiotic activity), by overlaying with an appropriate cell layer, optionally including an enzyme to cleave the linker binding the substrate to the solid support.

Another application utilizes proteins as electrical components. For example, arrays of proteins can form wires (e.g., where the proteins are electrically conductive), transistors/capacitors/gates (where the proteins or arrays of proteins have defined electrical properties), and the like. Similarly, an electrical input can be used to change the oxidation state of cofactors, giving an optical change. Protein memory devices can also be formed using the arrays of the invention. In another aspect, ligand activated cascades can function as switches.

In general, the detection methods described above involve a conformational change, e.g., in a biopolymer or biopolymer array, between a biopolymer and a bound analyte, between a biopolymer and a dye, between domains of a biopolymer, etc. Numerous variations of such detection methods can be produced by those of skill in the art for use in the context of biosensor devices and arrays, to detect non-nucleic acid analytes, e.g., small molecule analytes. The following examples are, therefore, provided to illustrate certain embodiments of the invention, and are not to be interpreted as limiting.

For example, antibody-antigen interactions interactions or lipocalin-substrate interactions can be used in the context of an optical biosensor for continuous detection of small molecule metabolites as well as proteins and peptides, e.g., in vivo or in vitro. Antibodies with binding affinity for a specified analyte are attached to the substrate, e.g., wall, of a sensing device. Also present within the confines of the device are large molecules (e.g., dextran, polyethyleneglycol, bovine serum albumin or other non-reactive proteins, etc.) that are covalently modified with the analyte of interest and a fluorescent moiety. A semi-permiable physical barrier (e.g., a molecular cut-off membrane) can be employed to insure retention of the analyte/fluroescence carrier molecule within the sensing device. When the sensor is placed in contact with a sample, e.g., in a collection vessel, or in an environmental sample in situ, or in a tissue of fluid in vivo, small molecules pass through the semi-permiable barrier and compete with the carrier molecule for binding sites on the antibody. If desired the sensing device is constructed to detect only molecules that are present free in solution and, thus, able to enter the detection range of the device through diffusion. Such a competition assay yields quantitative data that can be correlated with the concentration of the analyte in the sample. Similarly, by minor physical modifications in the device, the same principle can be used for detecting large proteins.

Another approach to developing a generalizable platform for detection of a wide range of analytes involves fusions of binding domains to an oxidase, e.g., glucose oxidase (GO). Such a platform takes advantage of the benefit of an electrochemically detectable signal generated by the oxidation, e.g., of glucose by glucose oxidase, combined with the broad spectrum of specificities available in binding molecules. Binding domains can be derived from antibodies, antibody domains, olfactory receptors, hormone receptors, lipocalins, enzymes and other binding molecules selected, e.g., using display systems. The oxidase-binding domain fusion protein(s) (e.g., GO-binding domain fusion proteins) is/are incubated with a sample (e.g., a biological fluid such as urine, plasma, saliva or blood) and binds the analyte of interest in the complex mixture. A sensor containing a surface derivatized with the analyte is used to capture any oxidase-binding domain fusion protein (e.g., GO-binding domain fusionprotein) with a free binding site. All unbound species are washed away and the bound portion is visualized by adding glucose. The signal created is inversely proportional to the concentration of the analyte. This type of sensor is particularly useful for detecting proteins and allowing standardized electrochemical detection of proteins and small molecule metabolites.

Another antibody based approach involves the use of a competitive enzyme linked immunosorbent assay (ELISA). A labeled analyte analogue is bound to an antibody (or other binding molecule, such as, a molecularly imprinted polymer, a receptor, or the like) immobilized, for example, on a surface or substrate, such as a chip, a plate, a bead, a membrane or other format for immobilization as described herein, e.g., in the context of biopolymer arrays. A detector, responsive to a signal generated by the marker is arranged to detect components of a sample that are not bound to the immobilized antibody. In the absence of the analyte of interest, the labeled analogue is bound to the antibody and signal is low. As the analyte concentration increases the analogue is displaced and the signal increases sigmoidally. Because this is an equilibrium measurement it can also be a real time continuous measurement.

In the case of a continuous sensor, the sensing region is isolated from the physiological or environmental sample, e.g., fluid, of interest by a semi-permiable physical barrier, as described above. The barrier can be selected such that molecules of the size of the analyte of interest would freely diffuse across the barrier, while molecules outside a specified range would be prohibited. In the case of applications in vivo, or in sensitive environments, the potentially toxic labeled analogue can be constructed (e.g., by polymerization or attachement to a pre-existing polymer such as dextran, dendrimers, beads, DNA, albumin, polyacrylomide, glucan, nylon, etc.) to be too large to traverse the barrier leading to functional isolation of the sensor from the surrounding sample or sample source. For in vivo applications, e.g., in a human subject, FDA approved polymers can be utilized.

Another approach for the continuous detection of an analyte (e.g., a protein of interest) involves detection of changes in a FRET signal. For example, in one variation, a surface or substrate is coated with an antibody, lipocalin (or any other binding protein) which is specific for the analyte of interest. This binding protein is labeled with a fluorophore, i.e. fluorescein. Analyte labeled with a second fluorophore that can act as part of a FRET pair with the fluorophore on the binding protein (i.e. rhodamine) is immobilized in proximity to the fluorescently tagged binding protein. The labeled analyte is attached to the surface by a tether (e.g., a polymer such as polyethylene glycol, a polypeptide, or peptide, or other linker molecule known to those of skill in the art) of defined length (which can be optimized empirically, taking into consideration effects on sensitivity related to the length and spacing of the tethered analyte). In the presence of the analyte of interest, the labeled analyte is displaced from the binding protein, leading to a decrease in FRET signal.

The above example relates, e.g., to the use of intact binding proteins, such as antibodies; however, fragments of such molecules, including minimal binding domains, e.g., Fab′ fragments, etc., are also favorably employed. For example, minimal binding domains from antibodies or other proteins (such as olfactory proteins, lipocalins, etc.), including artificial, e.g., shuffled, variants, that bind analytes of interest (either proteins or small-molecules) are constructed such that they can be easily labeled with two fluorophores which constitute a FRET pair (i.e., fluorescein and tetramethylrhodamine). Perturbations in protein structure upon binding of an analyte can be detected by changes in FRET signal. Affinity for the target analyte(s), as well as the extent of conformational change upon binding (which gives rise to the FRET signal) can be modified by directed evolution, e.g., by DNA shuffling. In addition, if desired, chemical modifications can be used to add fluorophores to the detector proteins. Alternatively, the minimal binding domain can be coupled to a fluorescent protein domain, e.g., in a fusion protein, eliminating the necessity of chemical modification steps. If desired, solid-phase binding domains, or other specific functionality can be engineered into the binding protein to facilitate its binding to a solid substrate or surface.

Any of the detection and/or analysis methods described above can be employed using a single (homogeneous) biopolymer, or using a heterogeneous array of functionally compatable biopolymers.

Optical Devices

To facilitate any of the methods or applications described above, luciferase, GFP, or other optically useful proteins can be optimized, e.g., by directed evolution procedures such as DNA shuffling, to emit light on application of an electrical or other stimuli, e.g., at defined wavlengths, upon binding of analytes, following conformational changes, etc., providing for lights, optical computing, bio-lasers, etc., in conjunction with the above described detection methods. Arrays of such proteins, including fusion proteins having a binding domain and an light emission domain can be used to form polychromatic displays, molecular posters, TVs, molecularly flat screen displays, or the like.

Similarly, light sensitive ocular (i.e., eye) proteins (derived from rods and/or cones, e.g., rhodopsin) can be optimized by directed evolution procedures to change or expand the wavelength range out to the ultraviolet or infrared range. Such proteins are useful, e.g., in conjunction with the light emitting detection methods described above, and can also be used, e.g., in an array format, in the production of molecular cameras and film.

Using Arrays of Biopolymers to Characterize Complex Samples.

The present invention relates to the production and utilization of libraries of nucleic acids and expression products, (RNA or polypeptide) as sensors for detecting a wide range of physical and biological stimuli. The diverse libraries of the invention are particularly useful for characterizing the constituents of complex samples, e.g., for medical diagnostics, environmental testing, biological and chemical warfare agent detection, metabolic profiling, drug screening, and the like. Typically, the libraries include variants of a nucleic acid or set of related nucleic acids, or expression products, e.g., protein variants encoded by the nucleic acids. Libraries of nucleic acid variants, e.g., libraries of diversified nucleic acid sequences, for example, shuffled DNA sequences encoding enzyme variants, have great potential in the realm of molecular detection. This potential is exploited in the methods of the invention by utilizing arrays of biopolymer libraries, such as libraries of diversified, e.g., shuffled, nucleic acids, or libraries of expression products encoded by diversified nucleic acid variants (e.g., shuffled nucleic acid variants), to detect a wide variety of biological, chemical and biochemical compounds.

The accuracy and sensitivity of sensing operations can be drastically increased by performing multiple assays, e.g., with enzymes of varying specificities and other properties. To accomplish this in an efficient and cost-effective manner, libraries of biopolymer variants, i.e., nucleic acid variants, or the expression products of nucleic acid variants, are logically or physically arrayed in any convenient manner, adapted to the specific test format or stimulus substrate of interest. The arrayed libraries are then used to rapidly determine (in parallel or rapid serial fashion) the activites engendered by the test stimulus or sample, generating a molecular signature or fingerprint corresponding to the stimulus or sample. By using libraries which include a large number of variants, it is possible to avoid the limitations in specificity of any single enzyme. Indeed, by using a large number of variants with overlapping specificities, it is possible to get an unambiguous fingerprint from the set of enzyme activities present in the array, even in the absence of a single component of the library with sufficient specificity for classical diagnostic applications.

In many cases, the library is sufficiently diverse that it can be used to simultaneously identify multiple stimuli, e.g., substrates, inhibitors, or effectors in a sample. This is accomplished by deconvoluting overlapping molecular fingerprints. In some applications, the library array consists exclusively of related enzymes capable of detecting stimuli of a particular class of molecules. Alternatively, the array consists of a variety of enzyme types, e.g., catalyzing a diverse set of reactions, to simultaneously detect several different molecules of interest, e.g., as are present in clinical fluid or biopsy samples, environmental samples, or the like.

In some preferred embodiments, the entire array is assayed using a single detection method. While this presents certain difficulties when using a heterogenous array, it can be accomplished, for example, by using a set of enzymes that give similar or similarly detected products, e.g., an array of oxidases that yield H₂O₂. Alternatively, a general electrochemical, microcalorimetric or optical detection method can be employed. Bifunctional detectors, having both binding or enzymatic activities, and reporter function, are particularly well suited to the library arrays of the invention.

The component biopolymers do not necessarily, themselves, transform the stimulus molecules for detection. In some cases, members of the arrayed libraries are differentially influenced, positively or negatively, by the presence of certain, e.g., inhibitor or effector, molecules. In this case, a particular inhibitor or positive effector generates a fingerprint on the array indirectly by influencing the catalytic reaction of the arrayed biopolymer.

In brief, diverse libraries of nucleic acids are produced by assembling natural or artificial variants of a nucleic acid or family of related nucleic acids, e.g., produced by recombining, mutagenizing, shuffling or other methods used to create variants of one or more parental nucleic acid. The diverse nucleic acids are arrayed, i.e., physically and/or logically organized, or expression products thereof are arrayed, to produce biopolymer arrays of interest. These arrays are optionally calibrated by contacting the array or a subset of the array to a known pattern forming stimulus (a molecule, light, heat, protons, etc.), to produce an array response (e.g., a signal or product). The arrays can then be contacted with unknown stimuli (e.g., unknown compounds) to produce a test array response. Comparison of the arrays response for the known stimulus to the arrays response for the test stimulus can be used to identify the test stimulus or stimuli.

Alternatively, instead of performing a comparative (e.g., diagnostic) function, the arrays can be used (e.g., in a re-usable format) to produce a product of interest. That is, the arrays can be thought of as reactors or reactor elements for producing products of interest.

Array responses can be characterized as molecular signatures or fingerprints (e.g., as in bar-coding strategies, diagnostic applications, monitoring applications, etc.), as products, or the like. Any signal from an array or biosensor can be stored in a database, typically by digitizing and storing the data in a computer or on computer readable media.

Such an indirect approach to detecting a stimulus is particularly useful, e.g., for the prediction of toxicity effects or efficacy of pharmaceutical agents. According to the methods of the invention, it is possible to perform a rapid spectrum test for, e.g., potential antibiotics or other pharmaceutical agents of interest. Combinatorial chemical libraries can also be rapidly screened against the array of variants to identify new specificities. Because the library array is likely to include functional space accessible to natural evolution, the array is also useful to predict, and counter, e.g., in the case of antibiotics, resistance mutations. For example, with respect to a potential antibiotic, a narrow spectrum (i.e., small number of array members responsive to the stimulus) can indicate that resistance is easily evolved.

Informatics Platform for Metabolomics

The value of capturing multiple signals in parallel from a systematically varied array of related proteins ensures a robust system with high precision and broad sensitivity. Each individual ‘pixel’ (spot of identical proteins in the array) will typically transduce a signal upon metabolite(s) binding to that particular protein. Some pixels are very specific for a given metabolite, whereas many confer promiscuous binding of different degrees to related metabolic compounds. The array, thus, encodes pixels corresponding to a high number of related proteins, each having its specific signature binding profile. The parallel multiplexed information gathered from such array will describe the combined metabolomic space, even if many, or even most, of the individual metabolites are unknown. The device generates a fingerprint of the tested sample, instead of a specific compound-by-compound reading, per se. The fingerprint can subsequently be convoluted to its individual substrate vectors, or alternatively (and more attractively) be used for a heuristically derived correlation with any number of physiological outputs or disease states. As the data, e.g., in a central database, increases, so does the significance of the prognostic and diagnostic outputs derived from the device.

There are several advantages to analyze the metabolome through pattern recognition instead of as individual signals. Firstly, the retrieved information can be parsed in a central database and the multidimensional information (one dimension for each pixel) can be used to cluster the sample with other samples from many representative disease states. The clustering can be done by neural net, partial least square or the use of any other statistical clustering tool. The output enables prognostic and diagnostic output from multiparameterized metabolite analysis in real time. Heuristic analysis of this type does not rely on an understanding of the disease model or identification of specific disease markers, but captures the full multidimensional spectrum of metabolite state in the sample as a function of binding to individual pixels in the array. In addition, the value and accuracy of the iterative database will increase as the accumulated data increases.

Secondly, for measuring individual metabolites, the individual metabolite does not have to be known or fully characterized, as long as it is structurally related to ensure binding to a specific subset of pixels in the array. A negative/positive validation of the array is done just once and all subsequent correlation is captured by the internal standard. Not only does the array identify absence/presence of the metabolite, but also all indirect effects of the altered metabolite levels is identified and used to validate the change.

Thirdly, internal standards can be included directly in the device. The quality of the array can be assessed by comparing the derived signal from pixels directed to the internal control with the signal from the pixels directed to the compounds of interest.

Exemplary Applications

The following exemplary applications are provided to illustrate the breadth of applicability of the methods of the invention, and should not be interpreted as limiting in scope or content.

Biosensors

The libraries of the invention can be arrayed to form a biosensor, e.g., a “nose,” which can be used to characterize/measure a broad spectrum of organic and non-organic molecules such as pheromones, chemical or biological warfare agents, hormones, proteins, etc. A sample of interest is added across the members of the array, which can be either a logical or a physical array of library members. Optionally, contact of the sample and array is followed by a washing step, depending on the precise format of the array. Binding is detected by a signal change (e.g., “on,” “off,” “increase,” “decrease,” etc.) at the binding array sites.

The biosensor is optionally composed of proteins or nucleic acids (DNA/RNA). Examples include olfactory receptor proteins, antibodies, lipocalins, phosphotransferases, permeases, transcription factors (e.g., small molecule regulated transcription factors), adhesion amplifiers, receptors and any other protein, DNA or RNA molecule that binds to a small molecule, protein, polymer or other compound to be asayed.

Binding can be measured by allosteric activation of enzymes, changes in redox potential, opening of an ion channel, any cellular signal transduction mechanism or by physical methods such as surface plasmon resonance.

In certain applications, a single library member, e.g., selected from a diversified library of variants, e.g., produced by shuffling or other diversification procedures, can itself be used as a “biosensor.” In cases where a single member of a library exhibits sufficient specificity and, either directly or indirectly, sufficient signal amplitude when linked to a suitable detector, a library member can be used outside the context of a library array to detect a stimulus. In most cases, such applications will be dedicated biosensors specific for a single, or small set of related compounds of interest, e.g., environmental toxins, biological warfare agents, serum components (e.g., glucose, ions, proteins, metabolic products, etc.), or the like.

Sample Profiling Using Enzyme Arrays

An array including activity diversity can be used to characterize constituents of a sample. In a manner analogous to the characterization of the functional limitations of individual members of a library using a set of substrates, the library as a group (or subgroup) can be utilized to profile the structural limitations, or components, of a sample or set of substrates. For example, a logical or physical array of enzymes can be used to acquire and characterize a fingerprint, i.e., a resulting array pattern or response, for a set of substrate molecules. The library arrays are conveniently categorized as either Single enzyme class (SEC) arrays or Multiple enzyme class (MEC) arrays. SEC arrays are composed of libraries of enzymes active on a certain class of molecular substrates. Typically, such libraries provide a complement of specificities that result in a fingerprint for each different substrate. By providing numerous, and, typically, overlapping, specificities, small differences can be detected between substrate molecules, enabling highly accurate diagnostic systems. Detection is readily performed using, e.g., microcalorimetry, electrochemical or optical detection methods, or physical partitioning on the nanoscale, e.g., in microfluidic or solid state devices.

In contrast, MEC arrays catalyze distinctly different reaction that may or may not give rise to a common product. For example, MEC arrays include enzymes which catalyze product formation from different classes of substrates (which can be related or unrelated), or catalyze the formation of different products from the same or related substrates. In some cases, MEC arrays are made up of multiple sets of diverse sub-libraries, including, e.g., SEC array libraries.

The use of multiple specificities from the library improves the accuracy of the procedure or system. Similarly, multiple simultaneous assays using the arrayed library improves the reliability. Libraries can be produced that detect a stimulus or class of stimuli either directly, e.g., by the catalytic conversion of the stimulus to a detectable product, or indirectly, e.g., by the modulating effect of the stimulus on the enzymatic activity of one or more library member. In some cases, cascade systems, e.g., in vivo activation of a reporter, can be used to increase sensitivity.

It will be apprecieated that enzyme libraries, such as the SEC and MEC libraries described above, can also be screened, e.g., in the context of an array, to identify a catalytic activity of interest (such as substrate binding, conversion of substrate to product, production of a compound of interest, and the like). An array of potential catalysts can be bound to a surface. The substrate of interest is applied to the array. Where catalysis is observed (heat/reduction potential/electrical change/colour, etc.) the protein is retrieved from storage and studied in more detail.

Medical or Environmental Diagnostics

Among the more widespread uses of the arrays of the invention are applications involved in medical, environmental and industrial diagnostic procedures and tools. For example, the libraries and arrays of the invention can be utilized in clinical biomedical applications, biomedical research, veterinary biomedical applications, and the like. Arrays useful in diagnostic (including prognostic) procedures include, e.g., libraries of enzymes (either SEC or MEC) involved in a cellular or metabolic pathway related to the physiologic or pathologic state defining the diagnosis in question. In other circumstances, libraries including antibodies, e.g., antibodies specific for one or multiple components (or products) of a metabolic or cellular pathway related to the diagnosis, or antibodies specific for one or multiple markers indicative of the diagnosis, can be used. In yet other circumstance, nucleic acid libraries rather than expression libraries are employed, e.g., to detect the presence or expression of nucleic acids correlated with the diagnosis. Numerous array formats are suitable and can be selected based on the specific diagnostic application. Examples of compounds that can be detected using biosensor arrays and/or array configurations of the invention include blood-glucose, ions, cytokines, cytokine receptors (at picogram/millileter sensitivity), antibodies, antigens (immunosensors), disease markers (e.g., as shown in Table 2), hormones (e.g., indicative of pregnancy, fertility, etc.), narcotics, steroids. viruses, bacteria, feedback regulators, food/beverage components, small molecule environmental compounds, metals (e.g., heavy metals), biological or chemical warfare agents, pharmaceutical agents, etc. Additional examples are provided throughout the specification.

In addition, in some biosensor formats, such non-chemical stimuli as temperature, sound, ultrasonic stimuli, mass, optical (i.e., light) and electrical (e.g., conductance) stimuli related to diagnostic and/or environmental state or condition can be detected.

For example, the arrays of the invention can be used for the detection of protein biomarkers associated with a disease, or other physiological condition, e.g., from cerebrospinal fluid, blood, biopsy samples. An array of binding proteins can be produced to provide detection of Alzheimers, hypertension, tumour identification etc. Direct detection of the presence or absence of specific protein variants (i.e., direct protein polymorphism/allele detection) can also be performed.

While any of a variety of array and detection formats as described herein are applicable for medical diagnostic applications, to simplify administration of a diagnostic test, certain formats are favorably employed. For example, where proteins are entrapped in a tape/capsule/dipstick or the like, the component can be dropped into a container of material to be sampled (urine, blood. etc.). This can be used to provide a home pregnancy test, or any other diagnostic assay that can be developed, including those noted herein. Any of the other detection techniques described can be also used in this format. A capsule can be dropped into a sample, with the color change recording, e.g., glucose level, pregnancy state, drug usage, estrous cycle, etc.

Similarly, the arrays of the invention are useful in environmental diagnostic procedures and tools, i.e., procedures aimed directly or indirectly at the detection of one or more chemical composition in an environment. In this context, an environment is generally considered other than the subject of a medical (including veterinary) diagnostic procedure, i.e., other than a human or non-human animal. It will be understood, however, that the distinction between medical and environmental diagnostics is largely a matter of convenience and is not based on limitations either in the array format or subject or sample under consideration. For example, the diagnosis of plant pathogens in a crop growing in under field conditions, evaluation of laboratory animals utilized as monitors for environmental toxins or pathogens, and monitoring the metabolic status of fungal (e.g., yeast) or bacterial cultures growing in a fermentor, as well as numerous other diagnostic applications fall clearly within the purview of the present invention as the following examples illustrate.

Process controllers can be used in the context of an industrial process determine components present in the product flow or waste stream for the process. The arrays of the invention can be used for such purposes, for example by monitoring accumulation of desired products (e.g., metabolites, reaction products, etc.), by-products or contaminants produced during an industrial process such as fermentation, refining, chemical production, etc. The arrays can also be used for feedback control of a complex reaction (fermentation media adjustment, sulfur levels in oil crackers, dioxins/CO₂/SO₂, in combustion effluents, etc.). Detection of impurities can be performed using an array, such as detection of agents that cause catalyst poisoning or unwanted byproducts (unwanted enantiomers/isomers, etc.).

The arrays can be also be used for environmental monitoring, e.g., detection of dangerous pollutants such as ozone/smog in cities (or, for that matter, more toxic compounds such as cyanogen bromide). Specific sensors can be placed, e.g., around a factory (e.g., designed to detect whatever the factory is making/storing), or can be placed in agricultural contexts to measure pesticides, methane, methyl bromide (e.g., in strawberry fields), etc. Similarly, the arrays can be utilized for environmental monitoring in such varied contexts as the military sector, security, agriculture etc.

In some embodiments, diverse libraries of biopolymers with improved specificities and activities relative to existing diagnostic reagents are produced by DNA shuffling or other nucleic acid diversification procedures. If desired, novel characteristics related to diagnostic activity or specificity can be identified, e.g., screened, from among the members of the library.

If the diversity of binding specificities in the array is appropriate, the array can be used to analyze a sample for multiple components in a sample. In cases where the binding specificities of materials to the array are known, array positions can be directly tied to a specific chemical signal. In the case of uncharacterized proteins, the arrays would be challenged with various stimuli and the pattern of response would be recorded. With multiple challenges, a map of responses could be derived empirically which would characterize the array. For each array design, a specific pattern of responses corresponds to a particular chemical “signature.” This can be trained into the imaging/analysis system and used to analyze replicate arrays. This is useful for disease diagnosis, nutrient analysis and can lead to a better understanding of diseases and methods of treatment.

The following list provides exemplary disease conditions, and putative markers amenable to detection using the biosensors of the present invention. It will be understood that this list is far from exhaustive, and is presented merely to provide a subset of illustrative examples.

TABLE 2
EXEMPLARY DISEASE CONDITIONS AND MARKERS

| Disease or Disease State | Markers |
|---|---|
| Congestive heart failure | N-acetylaspartate, creatine, choline, |
| | myosinositolm, serum uric acid, serum |
| | creatinine |
| Myocardial fibrosis | plasma procollagen type III amino- |
| | terminal peptide |
| Cardiotoxicity | brain natriuretic peptide (BNP) |
| Cancer | Prostate Specific Antigen, other cancer |
| | specific antigens, altered blood cell |
| | count, e.g., white blood cell, |
| | platelets, etc. |
| Brain/CNS function | atrial natriuretic peptide, brain |
| | natriuretic peptide, quinolinic acid, |
| | pyruvate |
| Endothelial function | palmitic (16:0) and palmitoleic (16:1) |
| | acids, linoleic acid (18:2 n6) and |
| | HDL-cholesterol, alfa-linolenic acid |
| | (18:3 n3) |
| Atherosclerosis | F(2)-isoprostanes, arachidonic acid, |
| | homocysteine |
| Ischaemia | (HCY), serum vitamin C concentration, |
| | hypoxanthine |
| Ischaemia-reperfusion injury | |
| Inflammatory vascular disease | |
| Peripheral arterial disease | |
| Hepatitis/liver function | bilirubin, hyaluronic acid (HA), |
| | tyrosine, branched-chain amino acids, |
| | aromatic amino acids, urinary |
| | porphyrins |
| Renal function | creatinine, creatine, uric acid, and p- |
| | aminohippuric acid. Urea or blood |
| | urea nitrogen (BUN), Bicarbonate, |
| | glucose |
| Bone formation/resorption/ | urinary pyridinoline, urinary de- |
| breakdown | oxypyridinoline, hydroxyproline, serum |
| | or urine calcium, serum or urine |
| | phosphate, Parathyroid hormone, 1,25- |
| | dihydroxyvitamin D3, osteocalcin, and |
| | C-terminal type I procollagen peptide, |
| | YKL40 glycoprotein |
| Mental illness/depression | homovanillic acid, 5-hydroxyindole- |
| | acetic acid, and 3-methoxy-4-hydroxy- |
| | phenylflycol, dopamine, serotonin, |
| | and norepineophrine |
| Skin | mycophenolate mofetil, mycophenolic |
| | acid |
| Multiple sclerosis | nitric oxide, IL6, corticosterone, serum |
| | amyloid A protein, creatine protein |
| Rheumatoid Arthritis | IL-1, cellular caspase inhibitory protein, |
| | vascular cell adhesion molecule 1, |
| | metalloproteinases, F2 isoprostanes, |
| | prostoglandin F(2α), prostoglandin |
| | E(2), dihydroxyvitamin D |
| Allergy | histamine, eosinophil cationic protein |
| General health | N-acetyl-asparate, creatine, choline and |
| | myo-inositol, N-acetylcyesteine con- |
| | jugates of valproic acid in urine, |

Detection of Secondary Metabolites at Low Concentrations.

Many substances of pharmaceutical importance are present in the body and blood at pico- or femtomolar concentrations. These molecules are typically very highly regulated due to their potent modulatory activity, such that a 2 to 10 fold increase in concentration has a therapeutically relevant physiological effect. In general, these molecules can be catogorized into a small number of classes: corticosteroids, prostagladins, eicosanoids, and peptide hormones (e.g., insulin, substance P, etc.).

Quantification of these molecules in vivo is particularly difficult for a number of reasons. Firstly, the stereochemistry and positional isomers of otherwise identical molecules give very different physiological responses and must, therefore, be distinguished. Secondly, these molecules tend to be hydrophobic and, therefore, their circulating blood concentration is very low, and frequently does not represent the physiologically relevant concentration at a relevant receptor or in a relevant membrane. Thirdly, the low concentration of these molecules leads to statistical sampling problems. For example, glucose monitoring is often performed on ˜1 μl of blood, but 1 μl of a femtomolar solution contains approximately a couple of hundred molecules of the analyte (for example: 10^{-15×10}⁻⁶/6×10²³). Accordingly, small variations in volume have huge affects on the number of molecules available for analysis, which leads to larger percentage variations in the signal. In addition, a couple of hundred detectable molecules provide a very small signal unless there is, e.g., a very large amplification and/or a very selective sensor.

The methods and devices of the present invention take advantage of the same system that an organism, e.g., a human body, uses to respond to these stimuli, by utilizing the binding properties of hormone receptors while modifying the output to yield an electrically or optically detectable signal.

In conjunction with the sensor platforms described herein, the following adaptations facilitate detection of hydrophobic analytes, such as the steroids described above. A comparatively large (1 ml) blood sample is collected, and contacted with a pre-concentration “pad” or membrane. As these molecules are hydrophobic, when passed over a suitable lipid bilayer membrane (or a polymer with similarly amphoteric characteristics), the analytes will concentrate in this membrane. Once the sample has been captured, the analytes are eluted as a concentrated bolus by an addition of detergents, organic solvents, chaotropes or the like. The eluted fraction is then applied to the array of sensing molecules.

Most of these hormones interact with either a G-protein coupled protein receptor or a nuclear hormone receptor. For example, G-protein coupled receptors are transmembrane proteins. For use in a sensing device, G-protein coupled receptors can be diversified and selected, e.g., shuffled, for activity and stability in a lipid bilayer membrane or inexpensive artificial membrane or membrane mimic, which allows diffusion of the proteins. In this manner, activity is retained that would otherwise be lost due to denaturation of the protein and adsorption onto the sensor surface.

Typically, these receptors respond to analyte binding in one of a small number of ways, e.g., for example they multimerize to form an open ion channel, or phosphorylate a kinase domain, thereby becoming catalytically active.

For example, in the case of receptors which act as ion channels, the receptor is manipulated, e.g., for screening and selection and under functional conditions, e.g., in a sensing device, in a hydrophobic environment such as a membrane, which is impermeable to ion flow, and placed over an electrode. In the presence of the analyte the channel opens allowing ions (Ca⁺⁺, K⁺ or the like) to flow to the electrode surface. This can be measured as a current flow. In order to optimize the signal, the receptor is selected for an unregulated opening (i.e., a single binding event leads to permanent channel opening).

In the case of kinases, e.g., tyrosine kinases, following phosphorylation in response to analyte binding, the kinase domain becomes catalytically active. The membrane can contain either an optically detectable (colorigenic, fluorogenic, luminogenic, etc.) substrate or an electrochemically active substrate, the product of which is detected at the underlying electrode. Alternatively, a different catalytic domain (e.g., with an simple assay such as a colorigenic, fluorogenic, luminogenic, etc. output) can be exchanged for the kinase domain, and optimized as desired.

Nuclear hormone receptors are small soluble intracellular proteins that change conformation upon ligand binding, this conformational change activates a DNA binding domain (often with dimerization) and initiates binding to a specific signal sequence in a target DNA modulating transcription of down-stream effector genes. Because these proteins bind to their ligand in solution they can be conveniently used in a multiplexed assay. All the receptors are pooled beneath a membrane, which is exposed to the collected sample. The analytes of interest diffuse through the membrane and bind to the receptor. The activated receptors then bind to their specific DNA sequence. Specific DNA sequences for all the receptors in the pool (along with, e.g., controls, etc.) are spotted on the surface of the detector at the base of the membrane bubble in a normal microarray format. The position at which the receptor binds is measured by standard methods (displacement of quenched fluorescent oligos, electrochemical change, etc.). The analyte is determined by the position of the response and the concentration by the level of response.

Alternatively, these nuclear hormone receptors can be engineered to form an active catalytic unit on dimerisation (either by bringing together the two halves of the protein or by conformational switch). Similarly, a FRET response is used to measure dimerization.

Sample Profiling to Predict Complex Properties.

A battery of easily detectable chemical and physical micro-tests can be used as predictors of application performance, e.g., of a pharmaceutical lead compound. For example, a battery of assays based, e.g., on effects on a library of protein variants of the invention, for example, due to binding, substrate conversion, conformational change, etc., can be used to generate an identifying profile or “fingerprint” for a compound. The correlation between performance, e.g., biochemical or physiological activity of the compound, and fingerprint data can then be determined. Minimal predictive fingerprint profiles are then utilized for screening a collection of compounds, such as a combinatorial chemical library for effects on a library of protein variants.

For example, multiple different factors affect the way a small molecule performs as a pharmaceutical agent or drug. Solubility, size, charge, positions of different active or structural groups, effect of pH on these or other properties, etc., as well as numerous other factors, all play a role in determining whether a pharmaceutical candidate molecule will in fact be suitable as a drug. Accordingly, a great deal of effort is expended in pharmaceutical discovery to develop an assay or model system that mimics the biological system of interest, such as a human being suffering from a specified disease. Such test systems tend to be low throughput due to the amount of care required to insure that the assay reproduces relevant features of the biological system.

The methods and arrays of the invention provide surrogate assays that can be performed at high-throughput and low cost. To illustrate, consider the example of a subtilisin protease. A single subtilisin protease performs differently at different pH, at different temperature, in different solvents, in the presence of different detergents, and on different substrates. None of these alone are good generalizable criteria for determining the ability of the protease to remove stains on clothes in a washing machine. However, all of the properties that are important for the desired application are measured in the simple assays corresponding to, e.g., pH, temperature, solvent or detergent conditions, and the like. Each of the simple assays can be treated as different dimensions, as can the more complex final assay. These dimensions can then be subjected to statistical analysis using such tools as principal component analysis. Significant components are those that distinguish between subtilisin variants that perform well in the wahing machine and those that do not. It is important that the statistical evaluation be able to distinguish between relevant and irrelevant information, in general the strength if the evaluation is increased by the presence of both good and poor performers in the sample set. The greater the number of variants present in an initial sample set, the greater the power of the statistical analysis, and, thus, the ability to distinguish between factors with good predictive value, and those with lesser predictive value.

Another example is to test candidate small molecules from a combinatorial (or in silico) library for their abilities to inhibit or stimulate multiple different enzymes, to inhibit or stimulate various signal transduction pathways, to induce protein toxicity indicators, and for their abilities to interact with an array of proteins, e.g., protein variants. The candidates are contacted with libraries of enzymes, signal transduction pathway components, etc., and binding is evaluated. Principle component analysis or other multivariate analysis method can be utilized as described in the example above to identify candidates that exhibit a binding or other interaction spectrum with members of the arrayed libraries, that correlate with compounds that perform well as drugs. In one approach, the small molecule candidates are evaluated in silico for interactions with a series of proteins, for example proteins for which the structure has been deduced by in silico protein folding algorithms, and the like

Drug Discovery

The action of a pharmaceutical compound is dependent on the changes that the compound causes to take place in the metabolism of an organism, e.g., within a cell. Both the positive effects, as well as undesired side effects, can often be related to changes in protein states (amount of protein present, protein localization, post-translational modification, etc.) which lead to other effects such as gene expression, opening or closing of ion channels, cell differentiation, etc. Development of new therapeutic agents often focuses on generating new compounds that maximize changes within a cell that are responsible for the desired therapeutic effects, while minimizing the cellular effects that lead to undesired side-effects. It is, therefore, of great interest to know the state of proteins within a cell upon treatment with a potential therapeutic candidate. Since mRNA levels often do not correlate well with corresponding protein levels, analysis of the protein composition within a cell, rather than simple expression profiling of RNA is a more relevant indication of the effect of a compound on a biological system, e.g., a cell or organism. In addition, mRNA expression analysis is prohibitively expensive to perform in high-throughput. In addition, many early cell-signaling events involve changes in protein localization or modification, e.g., phosphorylation, rather than the induction of RNA transcription.

The present invention provides methods for analyzing the protein complement of a cell. In one approach, extracts of cells treated with a potential therapeutic compound are bound in an array, e.g., on a microtiter plate treated to non-specifically bind protein. Protein can be bound by a variety of methods, depending on the substrate, including, e.g., electrostatic effects, or contact with functional groups on the solid surface which react with the protein to form covalent attachments. After the cellular proteins have been attached to the substrate, the soluble fraction of the extract is removed. If desired, the wells are washed, generally with a buffer, such as phosphate buffered saline (PBS). Additional blocking procedures, for example, using bovine serum albumin (BSA), can optionally be employed to prevent non-specific binding to sites on the surface of the substrate unoccupied by cellular proteins.

Each sample, e.g., each well on the microtiter plate or each of a set of duplicate wells containing the same cellular sample, is then contacted with a biosensor array of the invention. For example, using available instrumentation such as a Q-bot or other commercial instrumentation available to apply an array of proteins, e.g., with a range of binding specificities. Proteins suitable for this purpose include, antibodies with varying specificities for cellular proteins, receptors, lipocalins, other ligand-binding proteins, enzymes with protein or peptide substrates, and the like. A number of detection systems are suitable for evaluating binding of the biosensor library to the cellular proteins, as described in detail herein. For example, in the case of an antibody array, detection can be performed by such well established procedures as an enzyme-linked immunosorbent assay (ELISA). For additional details regarding application of ELISAs in a micro-array format, including performing multiple tandem assays in a single well of a microtiter plate, see, e.g., Mendoza et al. (1999) BioTechniques 27:778-788. To facilitate quantitation and/or detection, each of the antibodies contributing to the array can be generated in the same organism, e.g., a mouse. Where desired, e.g., to amplify signal, a secondary antibody conjugated to a reporter, e.g., biotin, IHRP, etc., can be applied. In this manner a diverse array of binding specificities can be used to characterize potential drug candidates. If desired, candidates can be analyzed in a rapid pre-screen using an array of binding proteins that is chosen to correlate with a particular pharmaceutical agent or class of agents.

Toxicity Screening

In another approach to drug discovery, arrays of gene variants of proteins relevant to detoxification (e.g., cytochrome P450s, including human P450s) are used to screen pharmaceutical candidates for potential toxicity.

Artificial Metabolism.

The biopolymer library arrays of the invention can be used to generate and identify novel metabolic pathways. For example the library arrays of the invention can be assayed for the ability to generate a signal or response indicative of utilization of a novel energy, e.g., carbon, source, thereby identifying library members with novel enzymatic activities relative to energy metabolism. Similarly, a wide range of enzymatic attributes can be elucidated, including substrate usage, cofactor usage, product generation. A significant benefit, of this approach to metabolic engineering, is the ability to engineer production hosts that are capable of generating the desired product while bypassing cellular metabolism in a way that minimally interferes with viability while simultaneously maximizing production. Likewise, the library arrays are useful in identifying enzymatic components that minimize crosstalk between metabolic pathways in order to eliminate a significant drain on input resources.

Protein Reagents for Research and Development Applications

Arrays of biopolymer libraries can be screened to identify characteristics that provide improved tools to accelerate discovery in biotechnology. For example, the arrayed libraries can be screened for novel restriction enzymes with, e.g., desired or improved specificity, activity, or reaction conditions. Similarly, the libraries can be used to identify DNA and RNA polymerases with increased fidelity, monomer specificity, or altered condition dependence. Many other enzyme activities, including ligases, endo- and exo- nucleases, recombinases, integrases, etc. can also be identified by screening the arrays of the invention.

For example, “designer restriction enzymes,” i.e., novel restriction enzymes with desired properties, e.g., novel or desired recognition sites, reaction conditions, etc., can be produced by directed evolution, e.g., shuffling. For example, one class of desirable restriction enzymes includes restriction enzymes that recognize long stretches of triplet repeats (e.g., as in many disease markers) and cleave them, but which do not cleave short stretches of such repeats. Restriction enzymes that recognize DNA superstructure (loops, triple helices, knots, histone or other protein induced superstructures, etc.) and cleave them can be produced This provides more choices in restriction enzyme design (e.g., entirely new classes of enzymes) for cloning flexibility, improved rates and specificities, formation of novel 3, 4, 5, 6, 7, and 8-base cutters, improved stability, etc. Restriction enzymes that cut and ligate specifically (e.g. recombinases like integrases/transposases: flp, cre/lox) can also be produced.

New polymerases can also be made, including those with high or low temp activity, high fidelity, low fidelity, even incorporation of unnatural bases, thermostability, Non-specific end addition (increased or decreased), activity or inactivity in the presence of impurities (e.g. humic acid, DMSO, ethanol, etc.), or the like.

Other enzymes/applications include new DNA ligases, enzymes with improved (or decreased) stability, thermostability, activity, improved activity for blunt-ended ligation, biotin ligase activity, co-factor regeneration, specificity, higher turnover, lower Km, disease diagnostics, etc.

Protein- or Site-Specific Enzymes

The directed evolution, e.g., shuffling, procedures described herein can also be used to produce protein modifying enzymes (e.g., proteases, lipases, glycosidases, etc.) or other proteins that modify proteins of interest, such as those linked to disease states with high sequence or structural selectivity. By evolving modifying enzymes specific for a protein of interest, it is possible to activate or destroy protein of interest, specifically modify target proteins to make them more visible/sensitive to the immune system, detect presence of specific protein variant (e.g., direct protein polymorphism/allele detection), etc., in a regulated manner.

Optimization of Fusion Proteins

In another application, libraries of fusion proteins physically or logically arrayed can be screened to identify fusion proteins with improved or optimized properties. Often, the linking of proteins (or polypeptides corresponding to a subportion of a protein) results in a decrease in one or more desirable activities due to the imperfect spatial arrangement of the domains of the fusion protein (or due to the addition of an affinity tag). Members of a library of diversified fusion proteins can be evaluated to identify fusion proteins with improved or optimized properties, e.g., increased catalytic activities, increased substrate binding, altered sensitivity to an inducer or inhibitor, etc.

Other Applications

Similarly, reporter genes/reporter systems can also be selected from a diversified library for any desired activity modification. Likewise, diagnostic antibodies can be produced (for extensive details on antibody shuffling see, e.g. Karrer et al., “Antibody Shuffling” WO 01/32712, published May 10, 2001) using the methods of the present invention, as described herein.

Making Diverse Libraries of Nucleic Acids

Diversifying Nucleic Acids

The nucleic acid variants comprising and/or encoding biosensor biopolymers, biosensor components, array components, or libraries of biopolymers of the invention can be produced or assembled in a number of ways. Random or selected sequences from one or more organism known or suspected to possess a particular trait relevant to the detection of a stimulus or set of stimuli can be arrayed for use in the present invention. Similarly, groups of naturally occurring related nucleic acids, or proteins, e.g., encoded by the related nucleic acids, can be arrayed for use in the methods of the invention. For example, multiple members of a gene family, and/or cognate genes from multiple species, are naturally occuring nucleic variants.

In many cases, however, improved precision, specificity, affinity, avidity, discrimination properties or the like, relative to that available through the use and expression of naturally occurring nucleic acids, is desirable. In such cases, the libraries of the invention are produced by diversification of naturally occurring or synthetic nucleic acids, to produce a library of nucleic acid variants. In some applications, e.g., detection of nucleic acids corresponding to a strain or substrain of bacteria or other organism, the diversified nucleic acid variants are themselves arrayed in the methods of the invention. Alternatively, expression products encoded by the diverse population of nucleic acids, are arrayed and serve as the bio-detectors of the invention.

A variety of diversity generating protocols are available and described in the art. The procedures can be used separately, and/or in combination to produce one or more variants of a nucleic acid or set of nucleic acids, as well variants of encoded proteins. Individually and collectively, these procedures provide robust, widely applicable ways of generating diversified nucleic acids and sets of nucleic acids (including, e.g., nucleic acid libraries), e.g., for use in the libraries and arrays of the present invention and for the engineering or directed evolution of nucleic acids, proteins, pathways, cells and/or organisms with new and/or improved characteristics.

While distinctions and classifications are made in the course of the ensuing discussion for clarity, it will be appreciated that the techniques are often not mutually exclusive. Indeed, the various methods can be used singly or in combination, in parallel or in series, to access diverse sequence variants.

Following diversification, any nucleic acids which are produced can be selected for a desired activity. In the context of the present invention, this can include testing for and identifying any activity that can be detected e.g., in an automatable format, by any of the assays in the art. A variety of related (or even unrelated) properties can be assayed for, using any available assay. Such properties include those which are useful to the format of the assay, such as enhanced stability of array members, orientation of protein binding, improved production, lower cost of manufacture, optimal activity of expressed members which comprise a tag, overexpression mutations, optimized protein folding, permanent enzyme secretion, improved operators, improved ribosome binding sites, avidity, selectivity, production of a detectable side product, and detection limit issues. Of course, activities of interest also include any activity relevant to the particular assay or array under developments, e.g., those which relate to the target of interest.

Descriptions of a variety of diversity generating procedures for generating modified nucleic acid sequences suitable for use in the biosensor arrays and applications described herein are found in the following publications and the references cited therein: Soong, N. et al. (2000) “Molecular breeding of viruses” Nat Genet 25(4):436-439; Stemmer, et al. (1999) “Molecular breeding of viruses for targeting and other clinical properties” Tumor Targeting 4:1-4; Ness et al. (1999) “DNA Shuffling of subgenomic sequences of subtilisin” Nature Biotechnology 17:893-896; Chang et al. (1999) “Evolution of a cytokine using DNA family shuffling” Nature Biotechnology 17:793-797; Minshull and Stemmer (1999) “Protein evolution by molecular breeding” Current Opinion in Chemical Biology 3:284-290; Christians et al. (1999) “Directed evolution of thymidine kinase for AZT phosphorylation using DNA family shuffling” Nature Biotechnology 17:259-264; Crameri et al. (1998) “DNA shuffling of a family of genes from diverse species accelerates directed evolution” Nature 391:288-291; Crameri et al. (1997) “Molecular evolution of an arsenate detoxification pathway by DNA shuffling,” Nature Biotechnology 15:436-438; Zhang et al. (1997) “Directed evolution of an effective fucosidase from a galactosidase by DNA shuffling and screening” Proc. Natl. Acad. Sci. USA 94:4504-4509; Patten et al. (1997) “Applications of DNA Shuffling to Pharmaceuticals and Vaccines” Current Opinion in Biotechnology 8:724-733; Crameri et al. (1996) “Construction and evolution of antibody-phage libraries by DNA shuffling” Nature Medicine 2:100-103; Crameri et al. (1996) “Improved green fluorescent protein by molecular evolution using DNA shuffling” Nature Biotechnology 14:315-319; Gates et al. (1996) “Affinity selective isolation of ligands from peptide libraries through display on a lac repressor ‘headpiece dimer’” Journal of Molecular Biology 255:373-386; Stemmer (1996) “Sexual PCR and Assembly PCR” In: The Encyclopedia of Molecular Biologv. VCH Publishers, New York. pp.447-457; Crameri and Stemmer (1995) “Combinatorial multiple cassette mutagenesis creates all the permutations of mutant and wildtype cassettes” BioTechniques 18:194-195; Stemmer et al., (1995) “Single-step assembly of a gene and entire plasmid form large numbers of oligodeoxy-ribonucleotides” Gene, 164:49-53; Stemmer (1995) “The Evolution of Molecular Computation” Science 270: 1510; Stemmer (1995) “Searching Sequence Space” Bio/Technology 13:549-553; Stemmer (1994) “Rapid evolution of a protein in vitro by DNA shuffling” Nature 370:389-391; and Stemmer (1994) “DNA shuffling by random fragmentation and reassembly: In vitro recombination for molecular evolution.” Proc. Natl. Acad. Sci. USA 91:10747-10751.

Mutational methods of generating diversity include, for example, site-directed mutagenesis (Ling et al. (1997) “Approaches to DNA mutagenesis: an overview” Anal Biochem. 254(2): 157-178; Dale et al. (1996) “Oligonucleotide-directed random mutagenesis using the phosphorothioate method” Methods Mol. Biol. 57:369-374; Smith (1985) “In vitro mutagenesis” Ann. Rev. Genet. 19:423-462; Botstein & Shortle (1985) “Strategies and applications of in vitro mutagenesis” Science 229:1193-1201; Carter (1986) “Site-directed mutagenesis” Biochem. J. 237:1-7; and Kunkel (1987) “The efficiency of oligonucleotide directed mutagenesis” in Nucleic Acids & Molecular Biology (Eckstein, F. and Lilley, D. M. J. eds., Springer Verlag, Berlin)); mutagenesis using uracil containing templates (Kunkel (1985) “Rapid and efficient site-specific mutagenesis without phenotypic selection” Proc. Natl. Acad. Sci. USA 82:488-492; Kunkel et al. (1987) “Rapid and efficient site-specific mutagenesis without phenotypic selection” Methods in Enzymol. 154, 367-382; and Bass et al. (1988) “Mutant Trp repressors with new DNA-binding specificities” Science 242:240-245); oligonucleotide-directed mutagenesis (Methods in Enzymol. 100: 468-500 (1983); Methods in Enzymol. 154: 329-350 (1987); Zoller & Smith (1982) “Oligonucleotide-directed mutagenesis using M13-derived vectors: an efficient and general procedure for the production of point mutations in any DNA fragment” Nucleic Acids Res. 10:6487-6500; Zoller & Smith (1983) “Oligonucleotide-directed mutagenesis of DNA fragments cloned into M13 vectors” Methods in Enzymol. 100:468-500; and Zoller & Smith (1987) “Oligonucleotide-directed mutagenesis: a simple method using two oligonucleotide primers and a single-stranded DNA template” Methods in Enzymol. 154:329-350); phosphorothioate-modified DNA mutagenesis (Taylor et al. (1985) “The use of phosphorothioate-modified DNA in restriction enzyme reactions to prepare nicked DNA” Nucl. Acids Res. 13: 8749-8764; Taylor et al. (1985) “The rapid generation of oligonucleotide-directed mutations at high frequency using phosphorothioate-modified DNA” Nucl. Acids Res. 13: 8765-8787 (1985); Nakamaye & Eckstein (1986) “Inhibition of restriction endonuclease Nci I cleavage by phosphorothioate groups and its application to oligonucleotide-directed mutagenesis” Nucl. Acids Res. 14: 9679-9698; Sayers et al. (1988) “Y-T Exonucleases in phosphorothioate-based oligonucleotide-directed mutagenesis” Nucl. Acids Res. 16:791-802; and Sayers et al. (1988) “Strand specific cleavage of phosphorothioate-containing DNA by reaction with restriction endonucleases in the presence of ethidium bromide” Nucl. Acids Res. 16: 803-814); mutagenesis using gapped duplex DNA (Kramer et al. (1984) “The gapped duplex DNA approach to oligonucleotide-directed mutation construction” Nucl. Acids Res. 12: 9441-9456; Kramer & Fritz (1987) Methods in Enzymol. “Oligonucleotide-directed construction of mutations via gapped duplex DNA” 154:350-367; Kramer et al. (1988) “Improved enzymatic in vitro reactions in the gapped duplex DNA approach to oligonucleotide-directed construction of mutations” Nucl. Acids Res. 16: 7207; and Fritz et al. (1988) “Oligonucleotide-directed construction of mutations: a gapped duplex DNA procedure without enzymatic reactions in vitro” Nucl. Acids Res. 16: 6987-6999).

Additional suitable methods include point mismatch repair (Kramer et al. (1984) “Point Mismatch Repair” Cell 38:879-887), mutagenesis using repair-deficient host strains (Carter et al. (1985) “Improved oligonucleotide site-directed mutagenesis using M13 vectors” Nucl. Acids Res. 13: 4431-4443; and Carter (1987) “Improved oligonucleotide-directed mutagenesis using M13 vectors” Methods in Enzymol. 154: 382-403), deletion mutagenesis (Eghtedarzadeh & Henikoff (1986) “Use of oligonucleotides to generate large deletions” Nucl. Acids Res. 14: 5115), restriction-selection and restriction-purification (Wells et al. (1986) “Importance of hydrogen-bond formation in stabilizing the transition state of subtilisin” Phil. Trans. R. Soc. Lond. A 317: 415-423), mutagenesis by total gene synthesis (Nambiar et al. (1984) “Total synthesis and cloning of a gene coding for the ribonuclease S protein” Science 223: 1299-1301; Sakamar and Khorana (1988) “Total synthesis and expression of a gene for the a-subunit of bovine rod outer segment guanine nucleotide-binding protein (transducin)” Nucl. Acids Res. 14: 6361-6372; Wells et al. (1985) “Cassette mutagenesis: an efficient method for generation of multiple mutations at defined sites” Gene 34:315-323; and Grundstrom et al. (1985) “Oligonucleotide-directed mutagenesis by microscale ‘shot-gun’ gene synthesis” Nucl. Acids Res. 13: 3305-3316), double-strand break repair (Mandecki (1986) “Oligonucleotide-directed double-strand break repair in plasmids of Escherichia coli: a method for site-specific mutagenesis” Proc. Natl. Acad. Sci. USA, 83:7177-7181; and Arnold (1993) “Protein engineering for unusual environments” Current Opinion in Biotechnology 4:450-455). Additional details on many of the above methods can be found in Methods in Enzymology Volume 154, which also describes useful controls for trouble-shooting problems with various mutagenesis methods.

Additional details regarding various diversity generating methods can be found in the following U.S. patents, PCT publications and applications, and EPO publications: U.S. Pat. No. 5,605,793 to Stemmer (Feb. 25, 1997), “Methods for In Vitro Recombination;” U.S. Pat. No. 5,811,238 to Stemmer et al. (Sep. 22, 1998) “Methods for Generating Polynucleotides having Desired Characteristics by Iterative Selection and Recombination;” U.S. Pat. No. 5,830,721 to Stemmer et al. (Nov. 3, 1998), “DNA Mutagenesis by Random Fragmentation and Reassembly;” U.S. Pat. No. 5,834,252 to Stemmer, et al. (Nov. 10, 1998) “End-Complementary Polymerase Reaction;” U.S. Pat. No. 5,837,458 to Minshull, et al. (Nov. 17, 1998), “Methods and Compositions for Cellular and Metabolic Engineering;” WO 95/22625, Stemmer and Crameri, “Mutagenesis by Random Fragmentation and Reassembly;” WO 96/33207 by Stemmer and Lipschutz “End Complementary Polymerase Chain Reaction;” WO 97/20078 by Stemmer and Crameri “Methods for Generating Polynucleotides having Desired Characteristics by Iterative Selection and Recombination;” WO 97/35966 by Minshull and Stemmer, “Methods and Compositions for Cellular and Metabolic Engineering;” WO 99/41402 by Punnonen et al. “Targeting of Genetic Vaccine Vectors;” WO 99/41383 by Punnonen et al. “Antigen Library Immunization;” WO 99/41369 by Punnonen et al. “Genetic Vaccine Vector Engineering;” WO 99/41368 by Punnonen et al. “Optimization of immunomodulatory Properties of Genetic Vaccines;” EP 752008 by Stemmer and Crameri, “DNA Mutagenesis by Random Fragmentation and Reassembly;” EP 0932670 by Stemmer “Evolving Cellular DNA Uptake by Recursive Sequence Recombination;” WO 99/23107 by Stemmer et al., “Modification of Virus Tropism and Host Range by Viral Genome Shuffling;” WO 99/21979 by Apt et al., “Human Papillomavirus Vectors;” WO 98/31837 by del Cardayre et al. “Evolution of Whole Cells and Organisms by Recursive Sequence Recombination;” WO 98/27230 by Patten and Stemmer, “Methods and Compositions for Polypeptide Engineering;” WO 98/27230 by Stemmer et al., “Methods for Optimization of Gene Therapy by Recursive Sequence Shuffling and Selection,” WO 00/00632, “Methods for Generating Highly Diverse Libraries,” WO 00/09679, “Methods for Obtaining in Vitro Recombined Polynucleotide Sequence Banks and Resulting Sequences,” WO 98/42832 by Arnold et al., “Recombination of Polynucleotide Sequences Using Random or Defined Primers,” WO 99/29902 by Arnold et al., “Method for Creating Polynucleotide and Polypeptide Sequences,” WO 98/41653 by Vind, “An in Vitro Method for Construction of a DNA Library,” WO 98/41622 by Borchert et al., “Method for Constructing a Library Using DNA Shuffling,” and WO 98/42727 by Pati and Zarling, “Sequence Alterations using Homologous Recombination;” WO 00/18906 by Patten et al., “Shuffling of Codon-Altered Genes;” WO 00/04190 by del Cardayre et al. “Evolution of Whole Cells and Organisms by Recursive Recombination;” WO 00/42561 by Crameri et al., “Oligonucleotide Mediated Nucleic Acid Recombination;” WO 00/42559 by Selifonov and Stemmer “Methods of Populating Data Structures for Use in Evolutionary Simulations;” WO 00/42560 by Selifonov et al., “Methods for Making Character Strings, Polynucleotides & Polypeptides Having Desired Characteristics;” WO 01/23401 by Welch et al., “Use of Codon-Varied Oligonucleotide Synthesis for Synthetic Shuffling;” and PCT/US01/06775 “Single-Stranded Nucleic Acid Template-Mediated Recombination and Nucleic Acid Fragment Isolation” by Affholter. As noted, array-based formats, particularly for expression of diversified products are also described in the references above.

In brief, several different general classes of sequence modification methods, such as mutation, recombination, etc. are applicable to the present invention and set forth, e.g., in the references above. Any number of these procedures can be utilized to generate diverse libraries suitable for the biosensor arrays, methods and applications described herein.

The following exemplify some of the different types of preferred formats for diversity generation in the context of the present invention, including, e.g., certain recombination based diversity generation formats.

Nucleic acids can be recombined in vitro by any of a variety of techniques discussed in the references above, including e.g., DNAse digestion of nucleic acids to be recombined followed by ligation and/or PCR reassembly of the nucleic acids. For example, sexual PCR mutagenesis can be used in which random (or pseudo random, or even non-random) fragmentation of the DNA molecule is followed by recombination, based on sequence similarity, between DNA molecules with different but related DNA sequences, in vitro, followed by fixation of the crossover by extension in a polymerase chain reaction. This process and many process variants is described in several of the references above, e.g., in Stemmer (1994) Proc. Natl. Acad. Sci. USA 91:10747-10751. Thus, in vitro diversification methods can be used to produce a diverse library of nucleic acids for use in the biosensor applications of the present invention, or from which libraries of enzymes or other proteins suitable for use as biosensors can be expressed.

Similarly, nucleic acids can be recursively recombined in vivo, e.g., by allowing recombination to occur between nucleic acids in cells. Many such in vivo recombination formats are set forth in the references noted above. Such formats optionally provide direct recombination between nucleic acids of interest, or provide recombination between vectors, viruses, plasmids, etc., comprising the nucleic acids of interest, as well as other formats. Details regarding such procedures are found in the references noted above. Thus, in vivo recombination methods can be utilized to produce a diverse library of nucleic acids suitable for use in the applications described herein.

Whole genome recombination methods can also be used in which whole genomes of cells or other organisms are recombined, optionally including spiking of the genomic recombination mixtures with desired library components (e.g., genes corresponding to the pathways of the present invention). These methods have many applications, including those in which the identity of a target gene is not known. Details on such methods are found, e.g., in WO 98/31837 by del Cardayre et al. “Evolution of Whole Cells and Organisms by Recursive Sequence Recombination;” and in, e.g., WO 00/04190 by del Cardayre et al., also entitled “Evolution of Whole Cells and Organisms by Recursive Sequence Recombination.” Such methods can be particularly favorable in generating libraries including variants from uncharacterized species, e.g., bacterial species capable of growth in an environment in which a compound of interest such as a toxin is present, and therefore likely to possess nucleic acids encoding proteins contributing to metabolism of the compound.

Synthetic recombination methods can also be used, in which oligonucleotides corresponding to targets of interest are synthesized and reassembled in PCR or ligation reactions which include oligonucleotides which correspond to more than one parental nucleic acid, thereby generating new recombined nucleic acids. Oligonucleotides can be made by standard nucleotide addition methods, or can be made, e.g., by tri-nucleotide synthetic approaches. Details regarding such approaches are found in the references noted above, including, e.g., WO 00/42561 by Crameri et al., “Olgonucleotide Mediated Nucleic Acid Recombination;” WO 01/23401 by Welch et al., “Use of Codon-Varied Oligonucleotide Synthesis for Synthetic Shuffling;” WO 00/42560 by Selifonov et al., “Methods for Making Character Strings, Polynucleotides and Polypeptides Having Desired Characteristics;” and WO 00/42559 by Selifonov and Stemmer “Methods of Populating Data Structures for Use in Evolutionary Simulations.”

In silico methods of recombination can be effected in which genetic algorithms are used in a computer to recombine sequence strings which correspond to homologous (or even non-homologous) nucleic acids. The resulting recombined sequence strings are optionally converted into nucleic acids by synthesis of nucleic acids which correspond to the recombined sequences, e.g., in concert with oligonucleotide synthesis/gene reassembly techniques. This approach can generate random, partially random or designed variants. Many details regarding in silico recombination, including the use of genetic algorithms, genetic operators and the like in computer systems, combined with generation of corresponding nucleic acids (and/or proteins), as well as combinations of designed nucleic acids and/or proteins (e.g., based on cross-over site selection) as well as designed, pseudo-random or random recombination methods are described in WO 00/42560 by Selifonov et al., “Methods for Making Character Strings, Polynucleotides and Polypeptides Having Desired Characteristics” and WO 00/42559 by Selifonov and Stemmer “Methods of Populating Data Structures for Use in Evolutionary Simulations.” Extensive details regarding in silico recombination methods are found in these applications. This methodology is generally applicable to the present invention in providing for generation of large and diverse nucleic sequence libraries in silico and/or the generation of corresponding nucleic acids or proteins. Such methods are of particular use in the development of, e.g., multifunctional proteins suitable for use in the biosensor arrays and applications of the present invention.

Many methods of accessing natural diversity, e.g., by hybridization of diverse nucleic acids or nucleic acid fragments to single-stranded templates, followed by polymerization and/or ligation to regenerate full-length sequences, optionally followed by degradation of the templates and recovery of the resulting modified nucleic acids can be similarly used. In one method employing a single-stranded template, the fragment population derived from the genomic library(ies) is annealed with partial, or, often approximately full length ssDNA or RNA corresponding to the opposite strand. Assembly of complex chimeric genes from this population is then mediated by nuclease-base removal of non-hybridizing fragment ends, polymerization to fill gaps between such fragments and subsequent single stranded ligation. The parental polynucleotide strand can be removed by digestion (e.g., if RNA or uracil-containing), magnetic separation under denaturing conditions (if labeled in a manner conducive to such separation) and other available separation/purification methods. Alternatively, the parental strand is optionally co-purified with the chimeric strands and removed during subsequent screening and processing steps. Additional details regarding this approach are found, e.g., in “Single-Stranded Nucleic Acid Template-Mediated Recombination and Nucleic Acid Fragment Isolation” by Affholter, PCT/US01/06775.

In another approach, single-stranded molecules are converted to double-stranded DNA (dsDNA) and the dsDNA molecules are bound to a solid support by ligand-mediated binding. After separation of unbound DNA, the selected DNA molecules are released from the support and introduced into a suitable host cell to generate a library enriched sequences which hybridize to the probe. A library produced in this manner provides a desirable substrate for further diversification using any of the procedures described herein.

Any of the preceding general recombination formats can be practiced in a reiterative fashion (e.g., one or more cycles of mutation/recombination or other diversity generation methods, optionally followed by one or more selection methods) to generate a more diverse set of recombinant nucleic acids.

Mutagenesis employing polynucleotide chain termination methods have also been proposed (see e.g., U.S. Pat. No. 5,965,408, “Method of DNA reassembly by interrupting synthesis” to Short, and the references above), and can be applied to the present invention. In this approach, double stranded DNAs corresponding to one or more genes sharing regions of sequence similarity are combined and denatured, in the presence or absence of primers specific for the gene. The single stranded polynucleotides are then annealed and incubated in the presence of a polymerase and a chain terminating reagent (e.g., ultraviolet, gamma or X-ray irradiation; ethidium bromide or other intercalators; DNA binding proteins, such as single strand binding proteins, transcription activating factors, or histones; polycyclic aromatic hydrocarbons; trivalent chromium or a trivalent chromium salt; or abbreviated polymerization mediated by rapid thermocycling; and the like), resulting in the production of partial duplex molecules. The partial duplex molecules, e.g., containing partially extended chains, are then denatured and reannealed in subsequent rounds of replication or partial replication resulting in polynucleotides which share varying degrees of sequence similarity and which are diversified with respect to the starting population of DNA molecules. Optionally, the products, or partial pools of the products, can be amplified at one or more stages in the process. Polynucleotides produced by a chain termination method, such as described above, are suitable substrates for any other described recombination format.

Diversity also can be generated in nucleic acids or populations of nucleic acids using a recombinational procedure termed “incremental truncation for the creation of hybrid enzymes” (“ITCHY”) described in Ostermeier et al. (1999) “A combinatorial approach to hybrid enzymes independent of DNA homology” Nature Biotech 17:1205. This approach can be used to generate an initial a library of variants which can optionally serve as a substrate for one or more in vitro or in vivo recombination methods. See, also, Ostermeier et al. (1999) “Combinatorial Protein Engineering by Incremental Truncation,” Proc. Natl. Acad. Sci. USA, 96: 3562-67; Ostermeier et al. (1999), “Incremental Truncation as a Strategy in the Engineering of Novel Biocatalysts,” Biological and Medicinal Chemistry, 7: 2139-44.

Mutational methods which result in the alteration of individual nucleotides or groups of contiguous or non-contiguous nucleotides can be favorably employed to introduce nucleotide diversity, e.g., for making biosensors and/or biosensor arrays. Many mutagenesis methods are found in the above-cited references; additional details regarding mutagenesis methods can be found in following, which can also be applied to the present invention.

For example, error-prone PCR can be used to generate nucleic acid variants. Using this technique, PCR is performed under conditions where the copying fidelity of the DNA polymerase is low, such that a high rate of point mutations is obtained along the entire length of the PCR product. Examples of such techniques are found in the references above and, e.g., in Leung et al. (1989) Technique 1:11-15 and Caldwell et al. (1992) PCR Methods Applic. 2:28-33. Similarly, assembly PCR can be used, in a process which involves the assembly of a PCR product from a mixture of small DNA fragments. A large number of different PCR reactions can occur in parallel in the same reaction mixture, with the products of one reaction priming the products of another reaction.

Oligonucleotide directed mutagenesis can be used to introduce site-specific mutations in a nucleic acid sequence of interest. Examples of such techniques are found in the references above and, e.g., in Reidhaar-Olson et al. (1988) Science, 241:53-57. Similarly, cassette mutagenesis can be used in a process that replaces a small region of a double stranded DNA molecule with a synthetic oligonucleotide cassette that differs from the native sequence. The oligonucleotide can contain, e.g., completely and/or partially randomized native sequence(s).

Recursive ensemble mutagenesis is a process in which an algorithm for protein mutagenesis is used to produce diverse populations of phenotypically related mutants, members of which differ in amino acid sequence. This method uses a feedback mechanism to monitor successive rounds of combinatorial cassette mutagenesis. Examples of this approach are found in Arkin & Youvan (1992) Proc. Natl. Acad. Sci. USA 89:7811-7815.

Exponential ensemble mutagenesis can be used for generating combinatorial libraries with a high percentage of unique and functional mutants. Small groups of residues in a sequence of interest are randomized in parallel to identify, at each altered position, amino acids which lead to functional proteins. Examples of such procedures are found in Delegrave & Youvan (1993) Biotechnology Research 11:1548-1552.

In vivo mutagenesis can be used to generate random mutations in any cloned DNA of interest by propagating the DNA, e.g., in a strain of E. coli that carries mutations in one or more of the DNA repair pathways. These “mutator” strains have a higher random mutation rate than that of a wild-type parent. Propagating the DNA in one of these strains will eventually generate random mutations within the DNA. Such procedures are described in the references noted above.

Other procedures for introducing diversity into a genome, e.g. a bacterial, fungal, animal or plant genome can be used in conjunction with the above described and/or referenced methods. For example, in addition to the methods above, techniques have been proposed which produce nucleic acid multimers suitable for transformation into a variety of species (see, e.g., Schellenberger U.S. Pat. No. 5,756,316 and the references above). Transformation of a suitable host with such multimers, consisting of genes that are divergent with respect to one another, (e.g., derived from natural diversity or through application of site directed mutagenesis, error prone PCR, passage through mutagenic bacterial strains, and the like), provides a source of nucleic acid diversity for DNA diversification, e.g., by an in vivo recombination process as indicated above.

Alternatively, a multiplicity of monomeric polynucleotides sharing regions of partial sequence similarity can be transformed into a host species and recombined in vivo by the host cell. Subsequent rounds of cell division can be used to generate libraries, members of which, include a single, homogenous population, or pool of monomeric polynucleotides. Alternatively, the monomeric nucleic acid can be recovered by standard techniques, e.g., PCR and/or cloning, and recombined in any of the recombination formats, including recursive recombination formats, described above.

Methods for generating multispecies expression libraries have been described (in addition to the reference noted above, see, e.g., Peterson et al. (1998) U.S. Pat. No. 5,783,431 “Methods for Generating and Screening Novel Metabolic Pathways,” and Thompson, et al. (1998) U.S. Pat. No. 5,824,485 Methods for Generating and Screening Novel Metabolic Pathways) and their use to identify protein activities of interest has been proposed (In addition to the references noted above, see, Short (1999) U.S. Pat. No. 5,958,672 “Protein Activity Screening of Clones Having DNA from Uncultivated Microorganisms”). Multispecies expression libraries include, in general, libraries comprising cDNA or genomic sequences from a plurality of species or strains, operably linked to appropriate regulatory sequences, in an expression cassette. The cDNA and/or genomic sequences are optionally randomly ligated to further enhance diversity. The vector can be a shuttle vector suitable for transformation and expression in more than one species of host organism, e.g., bacterial species, eukaryotic cells. In some cases, the library is biased by preselecting sequences which encode a protein of interest, or which hybridize to a nucleic acid of interest. Any such libraries can be provided as substrates for any of the methods herein described.

The above described procedures have been largely directed to increasing nucleic acid and/or encoded protein diversity. However, in many cases, not all of the diversity is useful, e.g., functional, and contributes merely to increasing the background of variants that must be screened or selected to identify the few favorable variants. In some applications, it is desirable to preselect or prescreen libraries (e.g., an amplified library, a genomic library, a cDNA library, a normalized library, etc.) or other substrate nucleic acids prior to diversification, e.g., by recombination-based mutagenesis procedures, or to otherwise bias the substrates towards nucleic acids that encode functional products. For example, in the case of antibody engineering, it is possible to bias the diversity generating process toward antibodies with functional antigen binding sites by taking advantage of in vivo recombination events prior to manipulation by any of the described methods. For example, recombined CDRs derived from B cell cDNA libraries can be amplified and assembled into framework regions (e.g., Jirholt et al. (1998) “Exploiting sequence space: shuffling in vivo formed complementarity determining regions into a master framework” Gene 215: 471) prior to diversifying according to any of the methods described herein.

Libraries can be biased towards nucleic acids which encode proteins with desirable enzyme activities. For example, after identifying a clone from a library which exhibits a specified activity, the clone can be mutagenized using any known method for introducing DNA alterations. A library comprising the mutagenized homologues is then screened for a desired activity, which can be the same as or different from the initially specified activity. An example of such a procedure is proposed in Short (1999) U.S. Pat. No. 5,939,250 for “Production of Enzymes Having Desired Activities by Mutagenesis.” Desired activities can be identified by any method known in the art. For example, WO 99/10539 proposes that gene libraries can be screened by combining extracts from the gene library with components obtained from metabolically rich cells and identifying combinations which exhibit the desired activity. It has also been proposed (e.g., WO 98/58085) that clones with desired activities can be identified by inserting bioactive substrates into samples of the library, and detecting bioactive fluorescence corresponding to the product of a desired activity using a fluorescent analyzer, e.g., a flow cytometry device, a CCD, a fluorometer, or a spectrophotometer.

Libraries can also be biased towards nucleic acids which have specified characteristics, e.g., hybridization to a selected nucleic acid probe. For example, application WO 99/10539 proposes that polynucleotides encoding a desired activity (e.g., an enzymatic activity, for example: a lipase, an esterase, a protease, a glycosidase, a glycosyl transferase, a phosphatase, a kinase, an oxygenase, a peroxidase, a hydrolase, a hydratase, a nitrilase, a transaminase, an amidase or an acylase) can be identified from among genomic DNA sequences in the following manner. Single stranded DNA molecules from a population of genomic DNA are hybridized to a ligand-conjugated probe. The genomic DNA can be derived from either a cultivated or uncultivated microorganism, or from an environmental sample. Alternatively, the genomic DNA can be derived from a multicellular organism, or a tissue derived therefrom. Second strand synthesis can be conducted directly from the hybridization probe used in the capture, with or without prior release from the capture medium or by a wide variety of other strategies known in the art. Alternatively, the isolated single-stranded genomic DNA population can be fragmented without further cloning and used directly in, e.g., a recombination-based approach, that employs a single-stranded template, as described above.

“Non-Stochastic” methods of generating nucleic acids and polypeptides are alleged in Short “Non-Stochastic Generation of Genetic Vaccines and Enzymes” WO 00/46344. These methods, including proposed non-stochastic polynucleotide reassembly and site-saturation mutagenesis methods be applied to the present invention as well. Random or semi-random mutagenesis using doped or degenerate oligonucleotides is also described in, e.g., Arkin and Youvan (1992) “Optimizing nucleotide mixtures to encode specific subsets of amino acids for semi-random mutagenesis” Biotechnology 10:297-300; Reidhaar-Olson et al. (1991) “Random mutagenesis of protein sequences using oligonucleotide cassettes” Methods Enzymol. 208:564-86; Lim and Sauer (1991) “The role of internal packing interactions in determining the structure and stability of a protein” J. Mol. Biol. 219:359-76; Breyer and Sauer (1989) “Mutational analysis of the fine specificity of binding of monoclonal antibody 51F to lambda repressor” J. Biol. Chem. 264:13355-60); and “Walk-Through Mutagenesis” (Crea, R; U.S. Pat. Nos. 5,830,650 and 5,798,208, and EP Patent 0527809 BI.

It will readily be appreciated that any of the above described techniques suitable for enriching a library prior to diversification can also be used to screen the products, or libraries of products, produced by the diversity generating methods.

Kits for mutagenesis, library construction and other diversity generation methods are also commercially available. For example, kits are available from, e.g., Stratagene (e.g., QuickChange™ site-directed mutagenesis kit; and Chameleon™ double-stranded, site-directed mutagenesis kit), Bio/Can Scientific, Bio-Rad (e.g., using the Kunkel method described above), Boehringer Mannheim Corp., Clonetech Laboratories, DNA Technologies, Epicentre Technologies (e.g., 5 prime 3 prime kit); Genpak Inc, Lemargo Inc, Life Technologies (Gibco BRL), New England Biolabs, Pharmacia Biotech, Promega Corp., Quantum Biotechnologies, Amersham International plc (e.g., using the Eckstein method above), and Anglian Biotechnology Ltd (e.g., using the Carter/Winter method above).

The above references provide many mutational formats, including recombination, recursive recombination, recursive mutation and combinations or recombination with other forms of mutagenesis, as well as many modifications of these formats. Regardless of the diversity generation format that is used, the nucleic acids of the invention can be recombined (with each other, or with related (or even unrelated) sequences) to produce a diverse set of recombinant nucleic acids, including, e.g., sets of homologous nucleic acids, as well as corresponding polypeptides.

Array Construction and Use

Many arraying methods are well known for arraying nucleic acids and proteins. General methods include spotting materials, chip-masking light synthetic techniques and many others. In addition to Ausubel, supra., reviews of nucleic acid arrays include Sapolsky et al. (1999) “High-throughput polymorphism screening and genotyping with high-density oligonucleotide arrays.” Genetic Analysis: Biomolecular Engineering 14:187-192; Lockhart (1998) “Mutant yeast on drugs” Nature Medicine 4:1235-1236; Fodor (1997) “Genes, Chips and the Human Genome.” FASEB Journal 11:A879; Fodor (1997) “Massively Parallel Genomics.” Science 277: 393-395; and Chee et al. (1996) “Accessing Genetic Information with High-Density DNA Arrays.” Science 274:610-614.

In addition to those in Ausubel, examples of protein-based arrays include various advanced immuno arrays (see, e.g., http://arrayit.com/protein-arrays/; Holt et al. (2000) “By-passing selection: direct screening for antibody-antigen interactions using protein arrays.” Nucleic Acids Research 28(15) E72-e72), superproteins arrays (see, e.g., http://www.jst.go.jp/erato/project/nts P/nts P.html), yeast two and other “n” hybrid array systems (see, e.g. Uetz et al. (2000) “A comprehensive analysis of protein-protein interactions in Saccharomyces cerevisiae” Nature 403, 623-627, and Vidal and Legrain (1999) “Yeast forward and reverse ‘n’-hybrid systems.” Nucleic Acids Research 27(4) 919-929); the universal protein array or “UPA” system (Ge et al. (2000) “UPA, a universal protein array system for quantitative detection of protein-protein, protein-DNA, protein-RNA and protein-ligand interactions.” Nucleic Acids Research, 28(2): E3-e3) and the like. Commercial companies such as Ciphergen (Freemont, Calif.); www.ciphergen.com, Beckman Coulter Inc. (Brea, Calif.); and others also provide commercial protein chip arrays.

Many publications relating generally to arrays and the use of arrays are available, including, e.g., Wilson et al. (2000) “Multiple differences in gene expression in regulatory V 24J Q T cells from identical twins discordant for type I diabetes” Proceedings of the National Academy of Sciences of the USA 97: 13, 7411-7416; Harrington et al. (2000) “Monitoring gene expression using DNA microarrays.” Microbiology 3: 3, 285-29; Kaminski et al. (2000) “Global analysis of gene expression in pulmonary fibrosis reveals distinct programs regulating lung inflammation and fibrosis.” Proceedings of the National Academy of Sciences of the USA 97:1778-1783; Lai et al. (2000) “Overexpression of human UDP-glucose pyrophosphorylase recuses galactose-1 phosphate uridyltransferase-deficient yeast.” Biochem Biophys Res Commun 10: 271, 392-400; Luthi-Carter et al. (2000) “Decreased expression of striatal signaling genes in a mouse model of Huntington's disease.” Human Molecular Genetics 9: 9, 1259-1271; Nau et al. (2000) “Technical Assessment of the Affymetrix Yeast Expression GeneChip YE6100 Platform in a Heterologous Model of Genes That Confer Resistance to Antimalarial Drugs in Yeast.” Journal of Clinical Microbiology 38: 5, 1901-1908; Warrington et al. (2000) “Gene-Expression Changes in Rol-Induced Cardiomyopathy” Proc. Natl. Acad. Sci. USA 9:4826-4831; and Webb et al. (2000) “Expression profiling of pancreatic cells: Glucose regulation of secretory and metabolic pathway genes” Proceedings of the National Academy of Sciences of the USA 97:11, 5773-5778; Anderson, R., et al. (2000) “A miniture integrated device for automated multistep genetic assays.” Nucleic Acids Research 28: 12, E60-e60; Wen-Hsiang Wen, et al (2000) “Comparison of TP53 Mutations Identified by Oligonucleotide Mircoarray and Conventional DNA Sequence Analysis.” Genomika 60:2716-2722; Lipshutz, R., et al. (2000) “Parallel Genotyping of Human SNPs Using Generic High-Density Oligonuclestide Tag Arrays.” Nature Biotechnology 10: 6, 853-860; Ahrendt et al. (1999) “Rapid p53 sequence analysis in primary lung cancer using an oligonucleotide probe array.” Proceedings of the National Academy of Sciences of the USA 96:7382-7387; Alon et al. (1999) “Broad patterns of gene expression revealed by clustering analysis of tumor and normal colon tissues probed by oligonucleotide arrays.” Proceedings of the National Academy of Sciences of the USA 96:6745-6750; Iwata et al. (1999) “Interleukin-1 (IL-1) Inhibits growth of cytomegalovirus in human marrow stromal cells: inhibition is reversed upon removal of IL-1.” Blood 94:572-578; Troesch et al. (1999) “Mycobacterium species identification and rifampin resistance testing with high-density DNA probe arrays.” Journal of Clinical Microbiology 37:49-55; Alon et al. (1999) “Broad Patterns of Gene Expression Revealed by Clustering Analysis of Tumor and Normal Colon Tissues Probed by Oligonucleotide Arrays.” Proceedings of the National Academy of Sciences of the USA 96:6745-6750; Bulyk et al. (1999) “Quantifying DNA-protein interactions by double-stranded DNA arrays.” Nature Biotechnology 17:573-577; Cheol-Koo Lee et al. (1999) “Gene Expression Profile of Aging and Its Retardation by Caloric Restriction.” Science 285: 1390-1393; Fambrough et al. (1999) “Diverse signaling pathways activated by growth factor receptors induce broadly overlapping, rather than independent, sets of genes.” Cell 97: 727-741; Galitski, et al. (1999) “Ploidy Regulation of Gene Expression.” Science 285: 251-254; Golub et al. (1999) “Molecular classification of cancer: class discovery and class prediction by gene expression monitoring.” Science 286:531-537; Harkin et al. (1999) “Induction of GADD45 and JNK/SAPK—Dependent apoptosis following inducible expression of BRACA1.” Cell 97:575-586; Jelinsky et al. (1999) “Global response of saccharomyces cerevisiae to an alkylating agent.” Proceedings of the National Academy of Sciences of the USA 96:1486-1491; Lee et al. (1999) “The Wilms Tumor Suppressor WT1 Encodes a Transcriptional Activator of amphiregulin.” Cell 98:663-673; Li et al. (1999) “Novel strategy yields candidate Gsh-1 homebox Gene Target Using Hypothalamus progenitor cell lines.” Developmental Biology 211:64-76; Madhani et al. (1999) “Effectors of a developmental mitogen-activated protein kinase cascade revealed by expression signatures of signaling mutants.” Proceedings of the National Academy of Sciences of the USA 96: 12530-12535; Mamatha et al. (1999) “A high-density probe array sample preparation method using 10- to 100- fold fewer cells.” Nature Biotechnology 17:1134-1136 1999; Lelivelt and Culbertson (1999) “Yeast Upf Proteins Required for RNA Surveillance Affect Global Expression of the Yeast Transcriptome.” Molecular and Cellular Biology 19: 6710-6719; Winzeler et al. (1999) “Functional Characterization of the S. cerevisiae Genome by Gene deletion and Parallel Analysis.” Science 285:901-906; Wyrick et al. (1999) “A Chromosomal landscape of nucleosome-dependent gene expression and silencing in yeast.” Science 402: 418-421; Fidanza, J. A., & McGall, G. H. (1999) “High-density nucleoside analog probe arrays for enhanced hybridization.” Nucleosides & Nucleotides 18:1293-1295; Glazer, M., et al. (1999) “High surface area substrates for DNA arrays Organic/Inorganic Hybrid Materials II.” Material Research 576:371-376; Lipshutz, R. J., et al. (1999) “High density synthetic oligonucleotide arrays.” Nature Genetics Chipping Forecast 21:20-24; Mazzola, L. T., et al. (1999) “Discrimination of DNA hybridization using chemical force microscopy.” Biophysical Journal 76: 2922-2933; Cargill, M., et al. (1999) “Characterization of single-nucleotide polymorphisms in coding regions of human genes.” Nature Genetics 22:231-238; Cho, R. J., et al. (1999) “Genome-wide mapping with biallelic markers in arabidopsis thaliana.” Nature Genetics 23:203-207; Gentalen, E, and Chee, M. (1999) “A Novel method for determining linkage between DNA sequences: hybridization to paired probe arrays.” Nucleic Acids Research 27:1485-1491; Hacia, J. G., et al. (1999) “Determination of ancestral alleles for human single-nucleotide polymorphisms using high-density oligonucleotide arrays.” Nature Genetics 22: 164-167; Halushka, M., et al. (1999) “Patterns of single-nucleotide polymorphisms in candidate genes for blood-pressure homeostasis.” Nature Genetics 22:239-247; Sapolsky, R, et al. (1999) “High-throughput polymorphism screening and genotyping with high-density oligonucleotide arrays.” Genetic Analysis: Biomolecular Engineering 14:187-192; Winzeler, E. A., et al. (1999) “Whole genome genetic-typing in yeast using high-density oligonucleotide arrays.” Parasitology 118:S73-S80; Winzeler, E. A., et al. (1999) “Functional Characterization of the S. cerevisiae Genome by Gene deletion and Parallel Analysis.” Science 285:901-906; Sapolsky, R, et al. (1999) “High-throughput polymorphism screening and genotyping with high-density oligonucleotide arrays.” Genetic Analysis: Biomolecular Engineering 14:187-192; “The Chipping Forecast” Nature Genetics 21(suppl): 1-60, 1999; Bassett DE Jr., et al. (1999) “Gene expression informaticsit's all in your mine.” Nature Genetics 21:51-55; Cheung VG, et al. (1999) “Making and reading microarrays.” Nature Genetics 21:15-19; Duggan DJ, et al. (1999) “Expression profiling using cDNA microarrays.” Nature Genetics 21:10-14; Iyer VR, et al. (1999) “The transcriptional program in the response of human fibroblasts to serum.” Science 283:83-87; Montagu J, and Weiner N. (1999) “Fluorescence array scanner employing a flying objective.” Science, Jan. 20, 1999; Yeung, G., et al. (1999) “Cloning of a Novel Epidermal Growth Factor Repeat Containing Gene EGFL6: Expressed in Tumor and Fetal Tissues.” Genomics 62:2; Mulero, J. J., et al. (1999) “IL1HY1: A Novel Interleukin-1 Receptor Antagonist Gene.” Biochemical and Biophysical Research Communications 263: 3; Mulero, J. J., et al. (1999) “CD39L4 is a secreted human apyrase, specific for the hydrolysis of nucleoside diphosphate.” J. Biol. Chem. 274, 20064-74;; Gunthard, et al. (1998) “Comparative Performance of High Density Oligonucleotide Sequencing of HIV Type 1 Pol from Clinical Samples.” -14:869-876; Hacia et al. (1998) “Strategies for mutational analysis of the large multiexon ATM gene using high-density oligonucleotide arrays.” Genome Research 8:1245-1258;

Zhu, H., et al. (1998) “Cellular Gene Expression Altered by Human Cytomegalovirus: Global Monitoring with Oligonucleotide Array.” Proceedings of the National Academy of Sciences of the USA 95:14470-14475; Cho, R. J., et al. (1998) “A Genome-Wide Transcriptional Analysis of the Mitotic Cell Cycle.” Molecular Cell 2:65-73; Cho, R. J., et al. (1998) “Parallel Analysis of Genetic Selections Using Whole Genome Oligonucleotide Arrays.” Proceedings of the National Academy of Sciences of the USA 95:3752-3757; DeSaizieu, A., et al. (1998) “Bacterial Transcript Imaging by Hybridization of Total RNA to Oligonucleotide Arrays.” Nature Biotechnology 16:45-48; Der, S. D., et al (1998) “Identification of genes differentially regulated by interferon x, B, or Y using oligonucleotide arrays.” Proceedings of the National Academy of Sciences of the USA 95:15623-15628; Gray, N. S., et al. (1998) “Exploiting Chemical Libraries, Structure, and Genomics in the Search for Kinase Inhibitors.” Science 281:533-537; Holstege, F. C. P., et al. (1998) “Dissecting the regulatory circuitry of a Eukaryotic Genome.” Cell 95:717-728; Paxton, W. A. et al. (1998) “Reduced HIV-1 Infectability of CD4(+) Lymphocytes from Exposed-Uninfected Individuals: Association with Low Expression of CCR5 and High Production of Beta-Chemokines.” Virology 244:66-73; Beecher, J. and Tirrell, D. (1998) “Synthesis of Protected Derivatives of 3-pyrrolylalanine.” Tetrahedron Letters 39:3927-3930; Forman, J. E., et al. (1998) “Thermodynamics of Duplex Formation and Mismatch Discrimination Onphotolithographically Synthesized Oligonucleotide Arrays.” Acs Symposium Series 682:206-228; Hacia, J. G., et al. (1998) “Enhanced high density oligonucleotide array-based sequence analysis using modified nucleoside triphosphates.” Nucleic Acids Research 26:4975-4982; Pirrung M. C., et al (1998) “Proofing of Photolithographic DNA Synthesis with 3′,5′-Dimethoxy-benzoinyloxycarbonyl-Protected Deoxynucleoside Phosphoramidites.” Journal of Organic Chemistry 63:241-246; Fan, J. B. (1998) “Accessing the Human Genome with High-Density DNA Arrays.” European Journal of Human Genetics 6:134-134; Gingeras, T. R., et al. (1998) “Simultaneous Genotyping and Species Identification Using Hybridization Pattern Recognition Analysis of Generic Mycobacterium DNA Arrays.” Genome Research 8:435-448; Gunderson, K. L., et al. (1998) “Mutation Detection by Litigation to Complete n-mer DNA Arrays.” Genome Research 8:1142-1153; Hacia J. G., et al. (1998) “Two Color Hybridization Analysis Using High Density Oligonuleotide Arrays and Energy Transfer Dyes.” Nucleic Acids Research 26(16):3865-6; Hacia, J. G., et al (1998) “Evolutionary Sequence Comparisons Using High-Density Oligonucleotide Arrays.” Nature Genetics 18:155-158; Wang, D. G., et al. (1998) “Large-Scale Identification, Mapping, and Genotyping of Single-Nucleotide Polymorphism's in the Human Genome.” Science 280:1077-1082; Winzeler, E. A., et al. (1998) “Direct Allelic Variation Scanning of the Yeast Genome.” Science 281:1194-1197; Lockhart, D. J. (1998) “Mutant yeast on drugs.” Nature Medicine 4:1235-1236; Cheung V C, et al. (1998) “Linage-equilibrium mapping without genotyping.” Nature Genetics 18:225-230; Ermolaeva O, et al. (1998) “Data management and analysis for gene expression arrays.” Nature Genetics 20:19-23; Jordan B. (1998) “Large-scale expression measurement by hybridization methods: from high density membranes to ““DNA chips”.” Journal of Biochemistry 124:251-258; Lemieux B, et al. (1998) “Overview of DNA chip technology.” Molecular Breeding 4:277-289; Rose S. D.(1998) “Application of a novel microarraying system in genomics research and drug discovery.” JALA 3:53-56; Welford, S. M. et al. (1998) “Detection of differentially expressed genes in primary tumor tissues using representational differences analysis coupled to microarray hybridization.” Nucleic Acid Research 26:3059-3065; Drmanac S., et al. (1998) “Accurate sequencing by hybridization for DNA diagnostics and individual genomics.” Nature Biotechnology 16: 54-58; Winters et al. (1997) “Human Immunodeficiency Virus Type 1 Reverse Transcriptase Genotype and Drug Susceptibility Changes in Infected Individuals Receiving Dideoxyinosine Monotherapy for 1 to 2 years.” Antimicrobial Agents and Chemotherapy 41:757-762; Wodicka, L. et al. (1997) “A Genome-Wide Expression Monitoring in Saccharomyces Cerevisiae.” Nature Biotechnology 15:1359-1367; Wodicka, L. et al. (1997) “Genome-Wide Expression Monitoring in Saccharomyces Cerevisiae” Nature Biotechnology 15:1359-1367; Bains, W. (1997) “Litigation Escalates for Patents on a Chip.” Nature Biotechnology 15:406-406; Beecher, Jody E., et al (1997) “Chemically Amplified Photolithography for the Fabrication of High Density Oligonucleotide Arrays Polymer/Material.” Science Engineering 76:597-598; Fodor, S. A. (1997) “Massively Parallel Genomics.” Science 277:393-395; Gette, W. and Kreiner, T. (1997) “Precision Scanning Technology for Complex Genetic Analysis.” American Laboratory 29:15-17; McGall, G. H., et al. (1997) “The efficiency of light-directed synthesis of DNA arrays on glass substrates.” Journal of the American Chemical Society 119(22): 5081-5090; Wallraff, G., et al. (1997) “DNA Sequencing on a Chip” Chemtech 27:22-32; Fan, J., et al. (1997) “Genetic Mapping: Finding and Analyzing Single-Nucleotide Polymorphisms with High-Density DNA Arrays.” American Journal of Human Genetics 61: 1601-1601; Hacia, J. G., et al. (1997) “Mutation Screening and Phylogenetic Analysis of Hereditary Breast Cancer Genes Using High Density Oligonucleotide Arrays.” American Journal of Human Genetics 61:361-361; Liu, W. W., et al. (1997) “Genetic Mapping: Finding and Analyzing Single-Nucleotide Polymorphisms with High-Density DNA Arrays.” American Journal of Human Genetics 61:1494-1494; Fodor S. (1997) “Genes, Chips and the Human Genome.” FASEB Journal 1 1:A879; Fodor, S. A. (1997) “Massively Parallel Genomics.” Science 277:393-395; DeRisi J. L., et al. (1997) “Exploring the metabolic and genetic control of gene expression on a genomic scale.” Science 278:680-686; Lashkari D. A., et al. (1997) Yeast microarrays for genome wide parallel genetic and gene expression analysis Proceedings of the National Academy of Sciences of the USA 94:13057-13062; Zhang L, et al. (1997) “Gene expression profiles in normal and cancer cells.” Science 276:1268-1272; Kozal et al. (1996) “Extensive Polymorphisms Observed in HIV-1 Clade B Protease Gene using High Density Oligonucleotide Arrays: implications for Therapy.” Nature Medicine 7:753-759; Lockhart, D. J., et al. (1996) “Expression Monitoring by Hybridization to High-Density Oligonucleotide Arrays.” Nature Biotechnology 14:1675-1680; Kreiner, T. (1996) “Rapid Genetic Sequence Analysis Using a DNA Probe Array System.” American Laboratory 28:39-43; McGall, G., et al. (1996) “Light-Directed Synthesis of High-Density Oligonucleotide Arrays Using Semiconductor Photoresists.” Proceedings of the National Academy of Sciences of the USA 93:13555-13560; Cronin, M., et al. (1996) “Detecting Cystic-Fibrosis Mutations by Hybridization to DNA-Probe.” Biologicals 24:209-209; Cronin, M. T., et al. (1996) “Cystic Fibrosis Mutation Detection by Hybridization to Light-Generated DNA Probe Arrays.” Human Mutation 7:244-255; Hacia, J. G., et al. (1996) “Detection of Heterozygous Mutations in BRCA1 using High Density Oligonucleotide Arrays and two-color Fluorescence Analysis.” Nature Genetics 14:441-447; Sapolsky, R. J. and Lipshutz, R. J. (1996) “Mapping Genomic Library Clones Using Oligonucleotide Arrays.” Genomics 33:445-456; Shoemaker D. D., et al. (1996) “Quantitative Phenotypic Analysis of Yeast Deletion Mutants using a Highly Parallel Molecular bar-coding Strategy.” Nature Genetics 14:450-456; Chee, M., et al. (1996) “Accessing Genetic Information with High-Density DNA Arrays.” Science 274:610-614; DeRisi J, et al. (1996) “Use of a cDNA microarray to analyze gene expression patterns in human cancer.” Nature Genetics 14:457-460; Pietu G, et al. (1996) “Novel gene transcripts preferentially expressed in human muscles revealed by quantitative hybridization of a high density cDNA array.” Genome Research 6:492-503; Schena M, et al. (1996) “Parallel human genome analysis: microarray-based expression monitoring of 1000 genes.” Proceedings of the National Academy of Sciences of the USA 93:10614-10619; Shalon D, et al. (1996) “A DNA microarray system for analyzing complex DNA samples using two-color fluorescent probe hybridization.” Genome Research 6:639-645; Drmanac S., et al. (1996) “Gene-representing cDNA clusters defined by hybridization of 57,419 clones from infant brain libraries with short oligonucleotide probes.” Genomics 37: 29-40; Milosavljevic A, et al. (1996) “Discovering distinct genes represented in 29,570 clones from infant brain cDNA libraries by applying sequencing by hybridization methodology.” Genome Research 6:132-141; Kozal et al. (1996) Nature Medicine 2(7): 753-759; Kozal et al. (1995) “Natural Polymorphism of HIV-1 Clade-B Protease Gene and Implications for Therapy Journal of Acquired Immune Deficiency Syndromes and Human Retrovirology 10:76; Mazzola, L. T. and Fodor, S. A. (1995) “Imaging Biomolecule Arrays by Atomic Force Microscopy.” Biophysical Journal 88:1653-1660; Lipshutz, R. J., et al. (1995) “Using Oligonucleotide Probe Arrays to Access Genetic Diversity.” BioTechniques 19:442-447; Schena M., et al. (1995) “Quantitative monitoring of gene expression patterns with complementary DNA microarray.” Science 270:467-470;Gallop, M., et al. (1994) “Applications of Combinatorial Technologies To Drug Discovery. 1Background and Peptide Combinatorial Libraries.” Journal of Medicinal Chemistry 37:1233-1251; Gordon, E., et al. (1994) “Applications of Combinatorial Technologies to Drug Discovery. 2.Combinatorial Organic-Synthesis, Library Screening Strategies, and Future-Directions.” Journal of Medicinal Chemistry 37:1385-1401; Jacobs, J. and Fodor, S. (1994) “Combinatorial Chemistry—Applications of Light-Directed Chemical Synthesis.” Trends In Biotechnology 12:19-26; Lipshutz R. J. and Fodor S. (1994) “Advanced DNA-Sequencing Technologies.” Current Opinion in Structural Biology 4:376-380; Lipshutz, R. J., et al. (1994) “DNA Sequence Confidence Estimation Genomics 19:417-424; Mazzola, L. T., and Fodor, S. (1994) “Direct Imaging of 2-Dimensional Biomolecule Arrays Using Atomic-Force.” Microscopy Biophysical Journal 66:A279-A279; Pease, A. C., et al. (1994) “Light-Generated Oligonucleotide Arrays for Rapid DNA Sequence Analysis.” Proceedings of the National Academy of Sciences of the USA 91:5022-5026; Drnanac S and Drmanac R. (1994) “Processing of cDNA and genomic kilobase-size clones for massive screening, mapping and sequencing by hybridization.” Biotechniques, 17: 328-336; Blondelguindi, S., et al. (1993) “Affinity Panning of A Library of Peptides Displayed on Bacteriophages Reveals the Binding-Specificity of BIP” Cell 75:771-728; Cho, C., et al. (1993) “An Unnatural Biopolymer.” Science 261:1303-1305; Fodor, S. A., et al. (1993) “DNA Sequencing by Hybridization” Proceedings of The Robert A. Welch Foundation 37th Conference on Chemical Research, 40 Years of the DNA Double Helix, Houston, Tex.; Fodor, S. A., et al. (1993) “Multiplexed Biochemical Assays with Biological Chips.” Nature 364:555-556; Lipshutz, R. (1993) “Likelihood DNA-Sequencing by Hybridization.” Journal of Biomolecular Structure & Dynamics 11:637-653; Sheldon, E., et al (1993) “DNA Hybridization.” Clinical Chemistry 39:718-719; Smith V., et al (1993) “Preparation and Fluorescent Sequencing of M13 Clones—Microtiter Methods.” Methods in Enzymology 218:173-187; Drmanac R., et al. (1993) “DNA sequence determination by hybridization: a strategy for efficient large-scale sequencing.” Science, 260:1649-52; Sheldon et al. (1993) Clinical Chemistry 39(4): 718-719; Rozsnyai, L., et al. (1992) “Photolithographic Immobilization of Biopolymers on Solid Supports.” Angewandte Chemie-International Edition in English 31:759-761; Dower, W. and Fodor, S. (1991) “The Search for Molecular Diversity 2. Recombinant and Synthetic Randomized Peptide Libraries.” Annual Reports in Medicinal Chemistry 26:271-280; Fodor, S., et al. (1991) “Light-Directed, Spatially Addressable Parallel Chemical Synthesis.” Science 251:767-773; Fodor et al. (1991) Science, 251: 767-777; Drmanac R., et al. (1989) “Sequencing of megabase plus DNA by hybridization: theory of the method.” Genomics 4:114-28.

Further details regarding array construction are found, e.g., in U.S. Pat. No. 5,143,854, to Pirrung et al.; in PCT/US98/11969 (WO98/56956); Fodor et al., PCT Publication No. WO 92/10092; and Hubbell U.S. Pat. No. 5,571,639.

Furthermore, companies such as Affymetrix (e.g., VLSIPS® arrays; Santa Clara, Calif.), Hyseq (Mountain View, Calif.), Research Genetics (e.g., the GeneFilters® microarrays; Huntsville Ala.), Axon Instruments (GenePix®; Foster City, Calif.), Operon (e.g., OpArrays®, Alameda, Calif.), Ciphergen (Freemont, Calif.); www.ciphergen.com, Beckman Coulter Inc. (Brea, Calif.), and many others provide diverse technologies for making physical arrays of nucleic acids, proteins and other molecules. For example, arrays have been used for Disease Management issues, Expression Analysis, GeneChip Probe Array Technologies, Genotyping and Polymorphism analysis, Spotted Array Technologies, and the like.

Several protocols for making arrays, e.g., of nucleic acids are also found on the internet, e.g., at http://www.protocolonline.net/molbio/DNA/dna_microarray.htm, in addition to the other references noted above. For example, this site provides relevant details regarding Protocols for Making Drosophila Arrays, PCR amplification of cDNAs for printing, polylysine slide preparation, “post-processing” and direct labeling of cdna probes, preparation of slides; preparation of dna samples, post-processing of arrays preparation of fluorescent DNA Probe from Yeast mRNA, preparation of fluorescent probe from human RNA preparation of fluorescent probe from E. coli RNA, preparation of fluorescent DNA probe from genomic DNA, cyanine dye HPLC purification, modified eberwine (“antisense”) RNA Amplification Protocol, hybridization of arrays, preparation of total RNA from cultured human cells preparation of PolyA+ mRNA from total Human RNA amplification and purification of cDNAs for microarray manufacture, microarray manufacture and processing, generating control mRNAs by In Vitro transcription; generating fluorescent cDNA controls by linear PCR, preparation of fluorescent probes from total human mRNA, cDNA microarray hybridization and washing, gene expression analysis with microarrays, mutation detection with oligonucleotide microarrays, comparative gene expression study using microarrays, microarray hybridization protocols, etc. Futher details regarding arraying methods are also found PCT/US01/01056, filed Jan. 10, 2001.

In one aspect, the invention includes putting proteins (e.g., expression products of shuffled nucleic acids) into arrays. In addition to the references noted above, proteomics approaches using various forms of protein arrays have been utilized by a number of investigators. For example, Nelson et al. (20000) “Biosensor chip mass spectrometry: a chip-based proteomics approach” Electrophoresis 21(6): 1155-63 (see also, Intrinsic Bioprobes, Inc., Tempe, Ariz. ibi@inficad.com) describe an interface of two general, instrumental techniques, surface plasmon resonance-biomolecular interaction analysis (SPR-BIA) and matrix-assisted laser desorption/ionization time-of-flight (MALDI-TOF) mass spectrometry, into a single concerted approach for use in the functional and structural characterization of proteins. Also, biomolecular interaction analysis—mass spectrometry (BIA-MS) is described for the detailed characterization of proteins and protein-protein interactions and the development of biosensor chip mass spectrometry (BCMS) as a chip-based proteomics approach. This approach can be adapted to the present invention by constructing appropriate protein arrays and following the methods noted by Nelson et al.

Similarly, Konig et al., (20000) “Multimicrobial sensor using microstructured three-dimensional electrodes based on silicon technology.” Anal Chem 72(9):2022-8 describe a system in which two microbial strains with different substrate spectra were immobilized separately within a single biosensor chip featuring four individually addressable platinum electrodes. These were sputtered onto the inner surface of four isolated pyramidal cavities (“containments”) micromachined on a silicon wafer. The biosensor chip was integrated into a flow-through system to measure the oxygen consumption of the immobilized microorganisms in the presence of assimilable analytes. The simple and mass-producible containment sensor exhibited good performance data: lower detection limit 0.1 mg/L naphthalene and 1 mg/L sensor-BOD; calibration range up to 30 mg/L; precision 3-6%; response time 2-3 min; service life up to 40 days; shelf life at 4° C. for 6 months, etc. The multimicrobial sensor was demonstrated by measuring ordinary municipal wastewater samples as well as various aqueous samples contaminated with PAH. Using chemometrical data analysis, the multimicrobial sensor provides a foundation for developing an “electronic tongue”. As adapted to the present invention, this array format utilizes shuffled components (e.g., shuffled or otherwise diversified proteins).

In another approach, Sonezaki (2000) “Analysis of the interaction between monoclonal antibodies and human hemoglobin (native and cross-linked) using a surface plasmon resonance (SPR) biosensor.” J Immunol Methods 238(1-2):99-106 describe a stable immuno-assay system for quantification of human hemoglobin as well as the interaction between various antibodies and Hb using a surface plasmon resonance (SPR) biosensor in a BlAcore with an immobilized anti-Hb antibody sensor chip. When polyclonal antibodies were used, the immuno-reactivity of purified and commercially available Hb decreased drastically with incubation times up to 14 h. This instability of immuno-reactivity of Hb is attributable to the conformational changes in Hb induced by oxidation. On the other hand, of the sixteen monoclonal antibodies tested, four antibodies (MSU-102,-103,-106 and -115) were found to maintain their immuno-reactivities at least up to 24 h. During long-term storage, however, the immuno-reactivity of Hb with these monoclonal antibodies decreased significantly. The chemical betabeta-cross-linking of Hb was effectively able to stabilize the structure of Hb and immuno-reactivity with monoclonal antibodies such as MSU-103 for periods at least up to 70 days. Therefore, the combination of specific monoclonal antibodies such as MSU-103 and a betabeta-cross-linked Hb standard was used for the quantification of Hb. As adapted to the present invention, antibodies can be shuffled for stability in an array format, and these shuffled antibodies used, e.g., as noted by Sonezaki et al.

Katerkamp et al. (1999) “Disposable optical sensor chip for medical diagnostics: new ways in bioanalysis.” Anal Chem 71(23):5430-5 describe an optical sensor system which is suited for medical point-of-care diagnostics. The system allows for several immunochemical assay formats and consists of a disposable sensor chip and an optical readout device. The chip is built up from a ground and cover plate with in- and outlet and, between, of an adhesive film with a capillary aperture of 50 microns. The ground plate serves as a solid phase for the immobilization of biocomponents. In the readout device, an evanescent field is generated at the surface of the ground plate by total internal reflection of a laser beam. This field is used for the excitation of fluorophor markers. The generated fluorescence light is detected by a simple optical setup using a photomultiplier tube. Because of the evanescent field excitation, washing or separation steps can be avoided. With this system the pregnancy hormone chorionic gonadotropin (hCG) was determined in human serum with a detection limit of 1 ng/mL. Recovery values were 86, 106, and 102% for 5, 50, and 100 ng/mL hCG, respectively. The SD in repeated measurements (n=10) was 5.6%. Furthermore, the feasibility of the system in competitive-type immunoassays was demonstrated for serum theophylline. A linear calibration curve of signal vs theophylline between 1 and 50 mg/L was obtained. Recovery values varied between 118% (10 mg/L) and 81.0% (20 mg/L). This approach can be adapted to the present invention using shuffled components on the solid phase.

Patton (2000) “Making blind robots see: the synergy between fluorescent dyes and imaging devices in automated proteomics” Biotechniques 28(5):944-8, 950-7 review various systems for examination of rare proteins, including fluorescence methods which deliver streamlined detection protocols, superior detection sensitivity, broad linear dynamic range and compatibility with modem microchemical identification methods such as mass spectrometry. Two general approaches to fluorescence detection of proteins are described: the covalent derivatization of proteins with fluorophores or noncovalent interaction of fluorophores either via the SDS micelle or through direct electrostatic interaction with proteins. One described approach for quantifying fluorescence is to use a photomultiplier tube detector combined with a laser light scanner. In addition, fluorescence imaging is performed using a charge-coupled device camera combined with a UV light or xenon arc source. Fluorescent dyes with bimodal excitation spectra may be broadly implemented on a wide range of analytical imaging devices, permitting their widespread application to proteomics studies and incorporation into semiautomated analysis environments. Any of these detection schemes can be used with the biosensors and biosensor arrays of the invention.

Rohlff (2000) “Proteomics in molecular medicine: applications in central nervous systems disorders.” Electrophoresis (2000) April 21(6):1227-34 describe proteomics appraoches relevant to CNS disorders. For example, bodily fluids such as cerebrospinal fluid (CSF) and serum are analysed at the time of presentation and throughout the course of the disease. Changes in the protein composition of CSF are indicative of altered CNS protein expression pattern with a causative or diagnostic disease link. Isolation strategies of clinically relevant cellular material such as laser capture micro-dissection, protein enrichment procedures and proteomic approaches to neuropeptide and neurotransmitter analysis are used to map out complex cellular interaction at a high level of detail. The resulting proteome database bypasses ambiguities of experimental models and facilitates pre- and clinical development of more specific disease markers and new selective fast acting therapeutics. Similarly, the present invention uses shuffled components to provide proteomic analysis. In another approach, de Lange (2000) “Detection of complement factor B in the cerebrospinal fluid of patients with cerebral autosomal dominant arteriopathy with subcortical infarcts and leukoencephalopathy disease using two-dimensional gel electrophoresis and mass spectrometry. Neurosci Lett 282(3): 149-52 investigated cerebrospinal fluid (CSF) from three CADASIL cases with known mutations in Notch-3 using two-dimensional gel electrophoresis. CSF from these patients was compared to that of six controls. A single spot in the protein maps of patients which was absent from all the controls was observed. In-gel tryptic digestion of this protein followed by mass spectrometric analysis of the tryptic fragments and a database search identified the spot as human complement factor B. In an approach of the present invention, similar approaches are used with shuffled components.

Alaiya (2000) “Cancer proteomics: from identification of novel markers to creation of artifical learning models for tumor classification.” Electrophoresis 21(6):1210-7describe an artificial learning models for tumor classification. The artificial learning approach has potential to improve tumor diagnosis and cancer treatment prediction. Similarly, neural networks and artificial learning processes can be used to correlate the empirical results of anything observed in an array-based system with any known disease or other condition.

Larsson et al. (2000) “Use of an affinity proteomics approach for the identification of low-abundant bacterial adhesins as applied on the Lewis(b)-binding adhesin of Helicobacter pylori.” FEBS Lett. 469(2-3):155-8 describe a carbohydrate-containing crosslinking probe to select bacterial surface adhesins for trypsin digestion, MALDI-TOF mass spectrometry and identification against genome sequence. Protein identification was obtained through the enrichment of approximately 300 fmol of adhesin from solubilized cells. Similar purification approaches can be used for the shuffled components of the present invention.

Thus, any of a variety of array configurations can be used in the systems herein. One common array format for use in the modules herein is a microtiter plate array, in which the array is embodied in the wells of a microtiter tray. Such trays are commercially available and can be ordered in a variety of well sizes and numbers of wells per tray, as well as with any of a variety of functionalized surfaces for binding of assay or array components. Common trays include the ubiquitous 96 well plate, with 384 and 1536 well plates also in common use.

In addition to liquid phase arrays, components can be stored or fixed in solid phase arrays, which are preferred in some of the applications noted herein. These arrays fix materials in a spatially accessible pattern (e.g., a grid of rows and columns) onto a solid substrate such as a membrane (e.g., nylon or nitrocellulose), a polymer or ceramic surface, a glass surface, a metal surface, or the like. Components can be accessed, e.g., by local rehydration (e.g., using a pipette or other fluid handling element) and fluidic transfer, or by scraping the array or cutting out sites of interest on the array. Alternately, the array can be used as an in-situ device component on its own, i.e., in many embodiments herein, the array is itself a product of interest.

While arrays are most often thought of as physical elements with a specified spatial-physical relationship, the present invention can also make use of “logical” arrays, which do not have a straightforward spatial organization. For example, a computer system can be used to track the location of one or several components of interest which are located in or on physically disparate components. The computer system creates a logical array by providing a “look-up” table of the physical location of array members. Thus, even components in motion can be part of a logical array, as long as the members of the array can be specified and located.

To facilitate production and operation of the devices and methods of the invention, populations of nucleic acids can be arranged into one or more physical or logical recombinant nucleic acid or expression product arrays. A duplicate of at least one of the one or more physical or logical recombinant nucleic acid arrays can be produced in the process of amplifying, sequencing, or expressing members of the nucleic acid array. Similarly, arrays of nucleic acids can be expressed and the expression products arrayed, in a manner that retains information about the position or type of nucleic acids in the parental nucleic acid array. The duplication process can be performed manually or in any automated or automatable format. In one typical embodiment, the system includes a nucleic acid or protein master array which physically or logically corresponds to positions of the nucleic acids and/or proteins in the reaction mixture array. This master array can be accessed as necessary, e.g., where access of reaction mixtures or other duplicated nucleic acid arrays is not feasible.

The following illustrates one exemplary automatable array copying format, e.g., for use in conjunction with diversified, e.g., shuffled, nucleic acid libraries. For example, arrays can be copied in an automated format to produce duplicate arrays, master arrays, amplified arrays and the like, e.g., where any operation is contemplated which could make recovery or detection of nucleic acids from an original array problematic (e.g. where a process to be performed destroys the original nucleic acids, e.g., recombination methods that change the nature of product nucleic acids as compared to starting nucleic acids), or where an elevated stability for the array would be helpful (e.g., where an amplified array can be produced to stabilize accessible copies of nucleic acids), or where a normalization of components (e.g., to provide similar concentrations of reactants or products) is useful for recombination, expression or analysis purposes. Copies can be made from master arrays, reaction mixture arrays or any duplicates thereof.

For example, nucleic acids can be dispensed into one or more master multiwell plates and, typically, amplified to produce a master array of elongated nucleic acids (e.g., by PCR) to produce an amplified array of elongated nucleic acids. The array copy system then transfers aliquots from the wells of the one or more master multiwell plates to one or more copy multiwell plates.

An array of reaction mixtures can be formed, e.g., by separate or simultaneous addition of an in vitro transcription reagent and an in vitro translation reagent to one or more copy multiwell plates (or other spatially organizing set of containers), or to a duplicate set thereof, to diversified nucleic acids.

In addition to adding reaction mixture components directly to arrays, reaction mixture components are commonly added to duplicate arrays of shuffled or otherwise diversified nucleic acids. For example, the reaction mixtures can be produced by adding in vitro transcription/translation reactants to a duplicate nucleic acid array, which is duplicated from a master array of the shuffled nucleic acids produced by spatially or logically separating members of a population of the shuffled nucleic acids.

Arraying techniques for producing both master and duplicate arrays from populations of shuffled or otherwise diversified nucleic acids can involve any of a variety of methods. For example, when forming solid phase arrays (e.g., as a copy of a liquid phase array, or as an original array), members of the population can by lyophilized or baked on a solid surface to form a solid phase array, or chemically coupled or printed (e.g., using ink-jet printing or chip-masking and photo-activated synthesis methods) to the solid surface. Similarly, population members can be converted from a solid phase to a liquid phase by rehydrating members of the population, or by cleaving chemically coupled members of the population of shuffled nucleic acids from the solid surface to form a liquid phase array. One or more physically separated logical or physical array member can be accessed from one or more sources of shuffled or otherwise diversified nucleic acids and moved to one or more array destination site (e.g., by pipetting into microtiter trays), where the one or more destinations constitute a logical array of the shuffled nucleic acids.

Individual members of an array can be copied in a number of ways. For example, members can be amplified and aliquots removed and placed in a duplicate array. Alternately, where the sequences of array members are deconvoluted (e.g., sequenced) copies can be produced synthetically and placed into copy arrays. Two preferred ways of copying array members are to use a polymerase (e.g., in amplification or transcription formats) or to use an in vitro nucleic acid synthesizer for copying operations. Typically, a fluid handling system will deposit copied array members in destination locations, although non-fluid based member transport (e.g., transfer in a solid or gaseous phase) can also be performed.

Expressing Diverse Libraries of Nucleic Acids

Making Nucleic Acids

Providing nucleic acids which are identified or generated as noted above (e.g., by the various diversity generation protocols), or which are to be diversified in the protocols noted above, generally takes one of two basic forms.

First, where a nucleic acid is selected which corresponds to a physically existant nucleic acid, that nucleic acid can be acquired by cloning, PCR amplication or other nucleic acid isolation methods as is common in the art. An introduction to such methods is found in available standard texts, including Berger and Kimmel, Guide to Molecular Cloning Techniques, Methods in Enzymology volume 152 Academic Press, Inc., San Diego, Calif. (Berger); Sambrook et al., Molecular Cloning—A Laboratory Manual (2nd Ed.), Vol. 1-3, Cold Spring Harbor Laboratory, Cold Spring Harbor, N.Y., 1989 (“Sambrook”) and Current Protocols in Molecular Biology, F. M. Ausubel et al., eds., Current Protocols, a joint venture between Greene Publishing Associates, Inc. and John Wiley & Sons, Inc., (supplemented through 1999) (“Ausubel”)). Examples of techniques sufficient to direct persons of skill through in vitro amplification methods, useful in identifying, isolating and cloning nucleic acid diversity targets, including the polymerase chain reaction (PCR) the ligase chain reaction (LCR), Qβ-replicase amplification and other RNA polymerase mediated techniques (e.g., NASBA), are found in Berger, Sambrook, and Ausubel, as well as Mullis et al., (1987) U.S. Pat. No. 4,683,202; PCR Protocols A Guide to Methods and Applications (Innis et al. eds) Academic Press Inc. San Diego, Calif. (1990) (Innis); Arnheim & Levinson (Oct. 1, 1990) C&EN 36-47; The Journal Of NIH Research (1991) 3, 81-94; (Kwoh et al. (1989) Proc. Natl. Acad. Sci. USA 86, 1173; Guatelli et al. (1990) Proc. Natl. Acad. Sci. USA 87, 1874; Lomell et al. (1989) J. Clin. Chem 35, 1826; Landegren et al., (1988) Science 241, 1077-1080; Van Brunt (1990) Biotechnology 8, 291-294; Wu and Wallace, (1989) Gene 4, 560; Barringer et al. (1990) Gene 89, 117, and Sooknanan and Malek (1995) Biotechnology 13: 563-564. Improved methods of cloning in vitro amplified nucleic acids are described in Wallace et al., U.S. Pat. No. 5,426,039. Improved methods of amplifying large nucleic acids by PCR are summarized in Cheng et al. (1994) Nature 369: 684-685 and the references therein, in which PCR amplicons of up to 40kb are generated. One of skill will appreciate that essentially any RNA can be converted into a double stranded DNA suitable for restriction digestion, PCR expansion and sequencing using reverse transcriptase and a polymerase. See, Ausubel, Sambrook and Berger, all supra.

Host cells can be transduced with nucleic acids of interest, e.g., cloned into vectors, for production of nucleic acids and expression of encoded molecules (these encoded molecules can be used, e.g., as controls to determine a baseline activity to compare encoded activities of a diverse library of nucleic acids to). In addition to Berger, Sambrook and Ausubel, a variety of references, including, e.g., Freshney (1994) Culture of Animal Cells, a Manual of Basic Technique, third edition, Wiley- Liss, New York and the references cited therein, Payne et al. (1992) Plant Cell and Tissue Culture in Liquid Systems John Wiley & Sons, Inc. New York, N.Y.; Gamborg and Phillips (eds) (1995) Plant Cell, Tissue and Organ Culture; Fundamental Methods Springer Lab Manual, Springer-Verlag (Berlin Heidelberg New York) and Atlas and Parks (eds) The Handbook of Microbiological Media (1993) CRC Press, Boca Raton, Fla. provide additional details on cell culture, cloning and expression of nucleic acids in cells.

Sources for physically existant nucleic acids include nucleic acid libraries, cell and tissue repositories, the NIH, USDA and other governmental agencies, the ATCC, zoos, nature and other sources familiar to one of skill. While these diverse sources provide many nucleic acids, there are many others which exist only as a result of computer algorithms as described above, or, even though existant, are difficult to acquire.

The second basic method for acquiring nucleic acids does not rely on the physical pre-existance of a nucleic acid. Instead, nucleic acids are generated sythetically, e.g., using well-established nucleic acid synthesis methods. For example, nucleic acids can be synthesized using commercially available nucleic acid synthesis machines which utilize standard solid-phase methods. Typically, fragments of up to about 100 bases are individually synthesized, then joined (e.g., by enzymatic or chemical ligation methods, or polymerase mediated recombination methods) to form essentially any desired continuous sequence. For example, the polynucleotides and oligonucleotides of the invention can be prepared by chemical synthesis using, e.g., the classical phosphoramidite method described by Beaucage et al., (1981) Tetrahedron Letters 22:1859-69, or the method described by Matthes et al., (1984) EMBO J. 3: 801-05., e.g., as is typically practiced in modern automated synthetic methods. According to the phosphoramidite method, oligonucleotides are synthesized, e.g., in an automatic DNA synthesizer, assembled and, optionally, cloned in appropriate vectors. In addition, essentially any nucleic acid can be custom ordered from any of a variety of commercial sources, such as The Midland Certified Reagent Company (mcrc@oligos.com), The Great American Gene Company (http://www.genco.com), ExpressGen Inc. (www.expressgen.com), Operon Technologies Inc. (Alameda, Calif.) and many others. Similarly, peptides and antibodies (useful in various embodiments noted below) can be custom ordered from any of a variety of sources, such as PeptidoGenic (pkim@ccnet.com), HTI Bio-products, inc. (http:/Iwww.htibio.com), BMA Biomedicals Ltd (U.K.), Bio.Synthesis, Inc., Research Genetics (Huntsville, Ala.) and many others.

Synthetic approaches to nucleic acid generation have the advantage of easy automation. Oligonucleotide synthesis machines can easily be interfaced with a digital system that instructs which nucleic acids to be synthesized (indeed, such digital interfaces are generally part of standard oligonucleotide synthesis devices). Similarly, ordering nucleic acids from commercial sources can be automated through simple computer programming and use of the internet (e.g., by having the user select nucleic acids which are desired and providing an automated ordering system), with provisions for user inputs (nucleic acid selection) and outputs (synthesis of nucleic acids which are ordered).

Synthetic approaches can also be used to automate simultaneous sequence acquisition and diversity generation, i.e., through “oligonucleotide shuffling” and related technologies (see also, “Oligonucleotide Mediated Nucleic Acid Recombination” by Crameri et al., filed Feb. 5, 1999 WO 00/42561, published Jul. 20, 2000; and “Use of Codon-Based Oligonucleotide Synthesis for Synthetic Shuffling” by Welch et al., filed WO 01/23401, published May 5, 2001; and “Methods for Making Character Strings, Polynucleotides and Polypeptides Having Desired Characteristics” by Selifonov and Stemmer, WO 00/42560, published Jul. 20, 2000. In these methods, nucleic acid oligonucleotides corresponding to multiple parental nucleic acids are synthesized, mixed and PCR assembled to produce recombinant nucleic acids which have subsequences corresponding to multiple parental nucleic acid types.

In general, of course, nucleic acids provided by either of these basic approaches can be used as substrates in the various diversity generation protocols noted above.

High-Throughput Cloning and Expression

In addition to in vitro transcription/translation, high throughput cloning and expression can be used to generated products to screen for product activity and/or to be arrayed in any of the various arraying methods noted herein. This approach has the advantage of expressing products in a system that is similar to the eventual intended expression site for many products (e.g., in cells).

Basic cloning methodology is set forth in Sambrook, Ausubel and Berger, supra, and this basic methodology can be used to produce proteins or nucleic acids of interest. In one high-throughput system, diversified nucleic acids (e.g., a shuffled DNAs) are transformed into cells. The cells are sorted (e.g., by FACS), e.g., by expression of a marker protein such as GFP, where the marker expression is encoded by a full-length copy of a corresponding nucleic acid, e.g., where the full-length nucleic acid also encodes a full-length product of interest. Cells that have been selected are transferred to a micro-chamber or array where they express the shuffled gene. The micro-chamber or array optionally contains a substrate for the shuffled protein whose optical properties (i.e. absorbance or fluorescence) are changed by catalysis by the enzyme. After a period of time, (e.g., ca. minutes to hours) the array of micro-chambers is “read” with a laser, CCD camera or other high density optical device. Those chambers in which the change in optical properties exceeds some threshold (i.e. a defining activity) are emptied, one into each well of a high density microtitre plate (96, 384, 1500 well etc), and the cells are then grown for the second assay. This provides a high-throughput format as a pre-screen for active clones.

In vitro Transcription/Translation

While simple cellular expression of nucleic acids to produce products to be arrayed can be performed, in one embodiment of the invention, libraries of nucleic acids produced by the various diversity generation methods set forth herein (shuffling, mutation, etc.) are transcribed (i.e., where the diverse nucleic acids are DNAs) into RNA and translated into proteins in vitro, which are screened by any appropriate assay or used as a biosensor arra as herein. Extensive discussions of such approaches are found in “Integrated Systems and Methods for Diversity Generation and Screening” by Bass et al., PCT/US01/01056, filed Jan. 10, 2001.

In brief, common in vitro transcription and/or translation reagents include reticulocyte lysates (e.g., rabbit reticulocyte lysates) wheat germ in vitro translation (IVT) mixtures, E coli lysates, canine microsome systems, HeLa nuclear extracts, the “in vitro transcription component,” (see, e.g., Promega technical bulletin 123), SP6 polymerase, T3 polymerase, T7 RNA polymerase (e.g., Promega # TM045), the “coupled in vitro transcription/translation system” (Progen Single Tube Protein System 3) and many others. Many of translation systems are described, e.g., in Ausubel, supra. as well as in the references below, and many transcription/translation systems are commercially available.

Methods of processing (transcribing and/or translating) diversified nucleic acids (shuffled, mutagenized, etc.) are provided. In the methods, a physical or logical array of reaction mixtures is provided, in which a plurality of the reaction mixtures include one or more member of a first population of nucleic acids (including shuffled, mutagenized or otherwise diversified nucleic acids). A plurality of the plurality of reaction mixtures further comprise an in vitro transcription or translation reactant. One or more in vitro translation products produced by a plurality of members of the physical or logical array of reaction mixtures is then detected. The physical or logical array of reaction mixtures produced by these methods are also a feature of the invention, i.e., when appropriate for use as biosensor elements as set for the herein.

Generally, cell-free transcription/translation systems can be employed to produce polypeptides from solid or liquid phase arrays of DNAs or RNAs as provided by the present invention. Several transcription/translation systems are commercially available and can be adapted to the present invention by the appropriate addition of transcription and or translation reagents to arrays of diversified nucleic acids, e.g., produced by shuffling target nucleic acids and arraying the resulting nucleic acids. A general guide to in vitro transcription and translation protocols is found in Tymms (1995) In vitro Transcription and Translation Protocols: Methods in Molecular Biology Volume 37, Garland Publishing, NY. Any of the reagents used in these systems can be flowed or otherwise directed into contact with nucleic acid array members, e.g., to produce arrays of transcribed or translated products to be used as biosensors.

Typically, in the present invention, in vitro transcription and/or translation reagents are added to an array (or duplicate thereof) that embodies the diverse populations of nucleic acids generated by diversity generating procedures. For example, where the nucleic acids of interest are plated on microtiter trays, the in vitro transcription/translation reagents are added to the wells of the trays to form arrays of reaction mixtures that individually comprise the in vitro transcription/translation reagents, the nucleic acids of interest and any other reagents of interest.

Several in vitro transcription and translation systems are well known and described in Tymms (1995), id. For example, an untreated reticulocyte lysate is commonly isolated from rabbits after treatment of the rabbits with acetylphenylhydrazine as a cell-free in vitro translation system. Similarly, coupled transcription/translation systems often utilize an E. coli S30 extract. See also, the Ambion 1999 Product Catalogue from Ambion, Inc (Austin Tex.).

A variety of commercially available in vitro transcription and translation reagents are commercially available, including the PROTEINscript-PROTM kit (for coupled transcription/translation) the wheat germ IVT kit, the untreated reticulocyte lysate kit (each from Ambion, Inc (Austin Tex.)), the HeLa Nuclear Extract in vitro Transcription system, the TnT Quick coupled Transcription/translation systems (both from Promega, see, e.g., Technical bulletin No. 123 and Technical Manual No. 045), and the single tube protein system 3 from Progen. Each of these available systems (as well as many other available systems) have certain advantages which are detailed by the product manufacturer.

In addition, the art provides considerable detail regarding the relative activities of different in vitro transcription translation systems, for example as set forth in Tymms, id.; Jermutus et al. (1999) “Comparison of E. Coli and rabbit reticulocyte ribosome display systems” FEBS Lett. 450(1-2):105-10 and the references therein; Jermutus et al. (1998) “Recent advances in producing and selecting functional proteins by using cell-free translation” Curr. Opin. Biotechnol. 9(5):534-48 and the references therein; Hanes et al. (1988) “Ribosome Display Efficiently Selects and Evolves High-Affinity Antibodies in vitro from Immune Libraries” PNAS 95:14130-14135 and the references therein; and Hanes and Pluckthun (1997) “In vitro Selection and Evolution of Functional Proteins by Using Ribosome Display.” Biochemistry 94:4937-4942 and the references therein.

For example, an untreated rabbit reticulocyte lysate is suitable for initiation and translation assays where the prior removal of endogenous globin mRNA is not necessary. The untreated lysate translates exogenous mRNA, but also competes with endogenous mRNA for limiting translational machinery.

Similarly, The PROTEINscript-PRO™ kit from Ambion is designed for coupled in vitro transcription and translation using an E. coli S30 extract. In contrast to eukaryotic systems, where the transcription and translation processes are separated in time and space, prokaryotic systems are coupled, as both processes occur simultaneously. During transcription, the nascent 5′-end of the mRNA becomes available for ribosome binding, allowing transcription and translation to proceed at the same time. This early binding of ribosomes to the mRNA maintains transcript stability and promotes efficient translation. Coupled transcription: translation using the PROTEINscript-PRO Kit is based on this E. coli model.

The Wheat Germ IVT™ Kit from Ambion, or other similar systems, is/are a convenient alternative, e.g., when the use of a rabbit reticulocyte lysate is not appropriate for in vitro protein synthesis. The Wheat Germ IVT™ Kit can be used, e.g., when the desired translation product comigrates with globin (approx. 12-15 kDa), when translating mRNAs coding for regulatory factors (such as transcription factors or DNA binding proteins) which may already be present at high levels in mammalian reticulocytes, but not plant extracts, or when an mRNA will not translate for unknown reasons and a second translation system is to be tested.

The TNT® Quick Coupled Transcription/Translation Systems (Promega) are single-tube, coupled transcription/translation reactions for eukaryotic in vitro translation. The TNT® Quick Coupled Transcription/Translation System combines RNA Polymerase, nucleotides, salts and Recombinant RNasin® Ribonuclease Inhibitor with the reticulocyte lysate to form a single TNT® Quick Master Mix. The TNT® Quick Coupled Transcription/Translation System is available in two configurations for transcription and translation of genes cloned downstream from either the T7 or SP6 RNA polymerase promoters. Included with the TNT® Quick System is a luciferase-encoding control plasmid and Luciferase Assay Reagent, which can be used in a non-radioactive assay for rapid (<30 seconds) detection of functionally active luciferase protein.

Many other systems are well known, well characterized and set forth in the references noted herein, as well as in other references known to one of skill. It will also be appreciated that one of skill can produce transcription/translation systems similar to those which are commercially available from available materials, e.g., as taught in the references noted above.

The methods of the invention can include in-line or off-line purification of one or more reaction product biosensor/array members. In line purification is performed as part of the transfer process from an in vitro transcription/translation reaction to a product detection or identification module, whereas off-line purification can be performed before or after transfer, or in a parallel module.

In any case, once expressed, proteins can be purified, either partially or substantially to homogeneity, according to standard procedures known to and used by those of skill in the art. Polypeptides of the invention can be recovered and purified from arrays by any of a number of methods well known in the art, including ammonium sulfate or ethanol precipitation, acid or base extraction, column chromatography, affinity column chromatography, anion or cation exchange chromatography, phosphocellulose chromatography, hydrophobic interaction chromatography, hydroxylapatite chromatography, lectin chromatography, gel electrophoresis and the like. Protein refolding steps can be used, as desired, in completing configuration of mature proteins. High performance liquid chromatography (HPLC) can be employed in final purification steps where high purity is desired. Once purified, partially or to homogeneity, as desired, the polypeptides may be used (e.g., as assay components, therapeutic reagents or as immunogens for antibody production).

In addition to the references noted supra, a variety of purification/protein folding methods are well known in the art, including, e.g., those set forth in R. Scopes, Protein Purification, Springer-Verlag, N.Y. (1982); Deutscher, Methods in Enzymology Vol. 182: Guide to Protein Purification, Academic Press, Inc. N.Y. (1990); Sandana (1997) Bioseparation of Proteins, Academic Press, Inc.; Bollag et al. (1996) Protein Methods, 2^{nd} Edition Wiley-Liss, NY; Walker (1996) The Protein Protocols Handbook Humana Press, NJ, Harris and Angal (1990) Protein Purification Applications: A Practical Approach IRL Press at Oxford, Oxford, England; Harris and Angal Protein Purification Methods: A Practical Approach IRL Press at Oxford, Oxford, England; Scopes (1993) Protein Purification: Principles and Practice 3^{rd} Edition Springer Verlag, NY; Janson and Ryden (1998) Protein Purification: Principles, High Resolution Methods and Applications, Second Edition Wiley-VCH, NY; and Walker (1998) Protein Protocols on CD-ROM Humana Press, NJ; and the references cited therein. Any of these approaches to protein purification can be used to purify proteins, e.g., for array synthesis.

As noted, those of skill in the art will recognize that after synthesis, expression and/or purification, proteins can possess a conformation substantially different from the native conformations of the relevant parental polypeptides. For example, polypeptides produced by prokaryotic systems often are optimized by exposure to chaotropic agents to achieve proper folding. During purification from, e.g., lysates derived from E. coli, the expressed protein is optionally denatured and then renatured. This is accomplished, e.g., by solubilizing the proteins in a chaotropic agent such as guanidine HCl.

In general, it is occasionally desirable to denature and reduce expressed polypeptides and then to cause the polypeptides to re-fold into the preferred conformation. For example, guanidine, urea, DTT, DTE, and/or a chaperonin can be added incubated with a transcription product of interest. Methods of reducing, denaturing and renaturing proteins are well known to those of skill in the art (see, the references above, and Debinski, et al. (1993) J. Biol. Chem., 268: 14065-14070; Kreitman and Pastan (1993) Bioconjug. Chem.,4: 581-585; and Buchner, et al., (1992) Anal. Biochem., 205: 263-270). Debinski, et al., for example, describe the denaturation and reduction of inclusion body proteins in guanidine-DTE. The proteins can be refolded in a redox buffer containing, e.g., oxidized glutathione and L-arginine. Refolding reagents can be flowed or otherwise moved into contact with the one or more polypeptide or other expression product, or vice-versa.

Various systems are also available for simultaneous synthesis and folding of complex proteins. For example, the control of redox potential, the use of helper proteins (from both bacterial and eukaryotic systems) and the like can be used to provide for improved cell free translation. In addition to the references noted above, additional details regarding cell free protein translation can be found at http://chemeng.stanford.edu/html/swartz.htm.

RNA or protein or other products of a translation reaction can be tagged with any available tag (biotin, His tag, etc.), and captured to an array position following expression, if desired. The products are optionally released, e.g., by cleavage of an incorporated cleavage site, or other releasing methods (salt, heat, acid, base, light, or the like). In alternate embodiments, products are free in solution or encapsulated in mini-reaction compartments such as inverted micelles or liposomes.

As noted it can be desirable to reconstitute expression products in liposomes, inverted micelles, or other lipid systems. Thus, the arrays or systems which include the arrays can include a source of one or more lipid. Typically this lipid is flowed into contact with the one or more polypeptide or other reaction product (or vice-versa), or into contact with the physical or logical array of reaction mixtures. Similarly, the lipid can be flowed into contact with one or more shuffled or mutagenized nucleic acids (or transcription products thereof), thereby producing one or more liposomes or micelles comprising the polypeptide or other reaction product, reaction mixture components, and/or nucleic acids.

Liposomes and related structures are particularly attractive systems for use in the present invention, because they serve to concentrate reagents of interest into small volumes and because they are amenable to FACS and other high-throughput methods. In addition to standard FACS methods, microfabricated FACSs for use in sorting cells and certain subcellular components such as molecules of DNA have also been described in, e.g., Fu, A. Y. et al. (1999) “A Microfabricated Fluorescence-Activated Cell Sorter,” Nat. Biotechnol. 17:1109-1111; Unger, M., et al. (1999) “Single Molecule Fluorescence Observed with Mercury Lamp Illumination,” Biotechniques 27:1008-1013; and Chou, H. P. et al. (1999) “A Microfabricated Device for Sizing and Sorting DNA Molecules,” Proc. Nat'l. Acad. Sci. 96:11-13. These sorting techniques utilizing microfabricated FACSs generally involve focusing cells using microchannel geometry and can be adapted to the present invention by the inclusion of a chip-based FACS system in the in vitro transcription/translation module of the system.

Data storage and Manipulation for Arrays and Array Products

During operation of the methods or devices of the invention, arrays are used, e.g., as sensors or as bioreactors to produce products of interest. Thus, in one significant aspect, the methods, devices or integrated systems herein have one or more product identification or purification modules. These product identification/purification modules identify and/or purify one or more members of the array or products of the array.

Common methods of assaying for array member or array product activities include any of those available in the art, including enzyme and/or substrate assays, cell-based assays, reporter gene expression, second messenger induction or signaling, etc.

In addition to array member identification, product identification or purification, and the like, such modules can also include an instruction set for discriminating between members of the array based upon detectable characteristics, such as a physical characteristic of the array members, bound test or control samples, array products, activities of members, bound components, products or reactants, and concentrations of the products or reactants. For example “hit picking” software is available which permits the user to select criteria to identify members of an array that display one or more activity which is sufficient to be of interest for further analysis, or to provide molecular signature information. For example, software for array analysis includes, e.g., Scanalyze® and NOMAD (see, e.g., http://www.microarrays.orWsoftware.html), as well as many other packages.

In general, the systems of the invention can include detection and/or selection modules which facilitate detection or selection of array members or array products. Such modules can include, e.g., an array reader which detects one or more member of the array of reaction products. Array readers are commercially available, generally constituting a microscope or CCD and a computer with appropriate software for identifying or recording information. In particular, array readers which are designed to interface with chips and standard microtiter trays and other common array systems are both commercially available.

Further, where a non-standard array format is used, or were non-standard assays are to be detected by the array reader, common detector elements can be used to form an appropriate array reader. For example, common detectors include, e.g., spectrophotometers, fluorescent detectors, microscopes (e.g., for fluorescent microscopy), CCD arrays, scintillation counting devices, pH detectors, calorimetry detectors, photodiodes, cameras, film, and the like, as well as combinations thereof. Examples of suitable detectors are widely available from a variety of commercial sources known to persons of skill.

Signals are preferably monitored by the array reader, e.g., using an optical detection system. For example, fluorescence based signals are typically monitored using, e.g., in laser activated fluorescence detection systems which employ a laser light source at an appropriate wavelength for activating the fluorescent indicator within the system. Fluorescence is then detected using an appropriate detector element, e.g., a photomultiplier tube (PMT), CCD, microscope, or the like. Similarly, for screens employing colorometric signals, spectrophotometric detection systems are employed which detect a light source at the sample and provide a measurement of absorbance or transmissivity of the sample. See also, The Photonics Design and Applications Handbook, books 1, 2, 3 and 4, published annually by Laurin Publishing Co., Berkshire Common, P.O. Box 1146, Pittsfield, Mass. for common sources for optical components.

In alternative aspects, the array reader comprises non-optical detectors or sensors for detecting a particular characteristic of the system. Such sensors optionally include temperature sensors (useful, e.g., when a product bound array component or array member produces or absorbs heat in a reaction, or when array is used in a reaction that involves cycles of heat as in PCR or LCR), conductivity, potentiometric (pH, ions), amperometric (for compounds that can be oxidized or reduced, e.g., O₂, H₂O₂, I₂, oxidizable/reducible organic compounds, and the like)S, mass (mass spectrometry), plasmon resonance (SPR/BIACORE), chromatography detectors (e.g., GC) and the like.

For example, pH indicators which indicate pH effects of receptor-ligand binding can be incorporated into the array reader, where slight pH changes resulting from binding can be detected. See also, Weaver, et al., Bio/Technology (1988) 6:1084-1089.

As noted, one conventional system carries light from a specimen field to a CCD camera. A CCD camera includes an array of picture elements (pixels). The light from the array specimen is imaged on the CCD. Particular pixels corresponding to regions of the array substrate (or beads, or plates, etc.) are sampled to obtain light intensity readings for each position. Multiple positions are processed in parallel and the time required for inquiring as to the intensity of light from each position is reduced. Many other suitable detection systems are known to one of skill.

Data obtained (and, optionally, recorded) by the detection device is typically processed, e.g., by digitizing image data and storing and analyzing the image in a computer system. A variety of commercially available peripheral equipment and software is available for digitizing, storing and analyzing a signal or image. A computer is commonly used to transform signals from the detection device into sequence information, reaction rates, molecular signatures (bar codes) or the like. Further details regarding arrays and probe tagging strategies is found, e.g., in Morris et al. EP 0799897A1 “Methods and Compositions for Selecting Tag Nucleic Acids and Probe Arrays” and in Shoemaker D. D., et al. (1996) “Quantitative Phenotypic Analysis of Yeast Deletion Mutants using a Highly Parallel Molecular bar-coding Strategy.” Nature Genetics 14:450-456.

Software for examining array patterns, determining reaction rates or monitoring formation of products by arrays are available or can easily be constructed by one of skill using a standard programming language such as Visualbasic, Fortran, Basic, Java, or the like, or can even be programmed into simple end-user applications such as Excel or Access. Software for array analysis is also commercially available, e.g., Scanalyze® and NOMAD (see, http://www.microarrays.org/software.html).

Any controller or computer which can incorporate a database of the invention optionally includes a monitor which is often a cathode ray tube (“CRT”) display, a flat panel display (e.g., active matrix liquid crystal display, liquid crystal display), or others. Computer circuitry is often placed in a box which includes numerous integrated circuit chips, such as a microprocessor, memory, interface circuits, and others. The box also optionally includes a hard disk drive, a floppy disk drive, a high capacity removable drive, and other elements for database storage. Inputing devices such as a keyboard, mouse or touch screen optionally provide for input from a user.

In addition to array readers, the product deconvolution module can include enzymes which convert one or more member of the array of reaction products into one or more detectable products, or substrates which are converted by the array of reaction products into one or more detectable products, or other features that provide for detection of product activity. For example, the array deconvolution/detection/data storage modules or others can include cells which produce a detectable signal upon incubation with members of the arrays, or products of the arrays, and reporter genes which are induced by one or more member of the array or products of the arrays. Similarly, the module can include promoters which are induced by one or more array member or product and, e.g., which directs expression of one or more detectable products. Enzyme or receptor cascades can be triggered which are induced by the one or more member of the array of reaction products, with any of the products of the cascade serving as a detectable event.

Any available system for detecting proteins or nucleic acids or other expression products (directly or indirectly) can be incorporated into the modules. Common product identification or purification elements include size/charge-based electrophoretic separation units such as gels and capillary-based polymeric solutions, as well as affinity matricies, plasmon resonance detectors (e.g.,BIACOREs), GC detectors, epifluorescence detectors, fluorescence detectors, fluorescent arrays, CCDs, optical sensors, FACS detectors, temperature sensors, mass spectrometers, stereo-specific product detectors, coupled H₂O₂ detection systems, enzymes, enzyme substrates, Elisa reagents or other antibody-mediated detection components (e.g., an antibody or an antigen), mass spectroscopy, or the like. The particular system to be used depends on the system at issue, the throughput desired and available equipment.

The product detection module can also include a substrate addition module which adds one or more substrate to a plurality of members of array or products of the array, e.g., where the product has an activity on the substrate. In this embodiment, the devices/array deconvolution modules can include a substrate conversion detector which monitors formation of a secondary product produced by contact between the substrate and one or more products. Formation of the product can be monitored directly or indirectly, or formation can be monitored by monitoring the substrate directly or indirectly (e.g., formation of the product can be monitored by monitoring loss of the substrate over time). Primary or secondary product formation can be monitored stereo selectively or non-selectively.

Formation of the secondary product can be monitored by detecting formation of peroxide, heat, entropy, changes in mass, charge, fluorescence, luminescence, epifluorescence, absorbance, or any of the other techniques previously noted or otherwise available for array member, array product or product activity detection which result from contact between a substrate and a product.

Commonly, product detectors can include a protein detector and the overall system will include protein purification means such as those noted for product purification generally. However, nucleic acids can also be members or products of the array, and can be similarly detected.

Array members can be moved into proximity to product identification modules, or vice versa. For example, the product identification module can perform an xyz translation of either the identification module or the array (e.g., by conventional robotics), thereby moving the product identification module proximal to the array of reaction products. Similarly, the one or more reaction product array members can be flowed into proximity to the product identification module. In-line or off-line purification systems can purify the one or more reaction product array members from associated materials.

Commonly detected array members or products include detection of or by: radiation, a polymer, a chemical moiety, a biopolymer, a nucleic acid, an RNA, a DNA, a protein, a ligand, an enzyme, a chemo-specific enzyme, a regio-specific enzyme, a stereo-specific enzyme, a nuclease, a restriction enzyme, a restriction enzyme which recognizes a triplet repeat, a restriction enzyme that recognizes DNA superstructure, a restriction enzyme with an 8 base recognition sequence, an enzyme substrate, a regio-specific enzyme substrate, a stereo-specific enzyme substrate, a ligase, a thermostable ligase, a polymerase, a thermostable polymerase, a co-factor, a lipase, a protease, a glycosidase, a toxin, a contaminant, a metal, a heavy metal, an immunogen, an antibody, a disease marker, a cell, a tumor cell, a tissue-type, cerebro-spinal fluid, a cytokine, a receptor, a chemical agent, a biological agent, a fragrance, a pheromone, a hormone, an olfactory protein, a metabolite, a molecular camera protein, a rod protein, a cone protein, a light-sensitive protein, a lipid, a pegylated material, an adhesion amplifier, a drug, a potential drug, a lead compound, a protein allele, a catalyst or the like.

The present invention also provides for array duplication. For example, secondary product arrays can be produced by re-arraying members of reaction products made using a first array, or the members of the first array, e.g., at a selected concentration of product members in the secondary product array. The selected concentration can be approximately the same for a plurality of product members in the secondary product array (sometimes all of the array members are plated at the same concentration, but it is also possible to plate members at different concentrations to provide multi-concentration datapoints, e.g., for kinetic analysis). This normalization of concentration simplifies analysis by product detection modules. Further details on array copy systems, including copying of product arrays, array normalization, and the like, are found in “Integrated Systems and Methods for Diversity Generation and Screening” by Bass et al., PCT/US01/01056, filed Jan. 10, 2001.

In addition to (or in place of ) actually re-arraying materials, detection modules (or a separate module) can include an instruction set for determining a correction factor which accounts for variation in product concentration at different positions in the relevant array. For example, where product concentrations are known, a concentration dependent correction can be applied to correct observed activity data.

### EXAMPLES

The following examples are provided by way of illustration and do not limit the invention in any way.

### Example 1

### Production of a Library of Transcription Regulator Variants

Twenty transcriptional regulators with binding activities for small organic molecules were identified in a search of publicly available databases, and are represented by the following Genbank accession numbers: A47078, CAA48174.1, BAA09883.1, CAA62584.1, S47095, CAB52211.1, P06519, AAA84988.1, AAC32451.1, CAA93242.1, AAD09866.1, AAC44567.1, AAC77386.1, BAA87867.1, BAA34177.1, AAD03979.1, AAB57638.1, BAA84117.1, A26804, and AAA26030.1. DNA corresponding to the above accession numbers is isolated, e.g., by purification from the appropriate bacterial strain or by amplification by a PCR using appropriate primers. The isolated DNA is fragmented, e.g., by any of the previously described techniques, and fragments from any or all of the isolated genes encoding transcriptional regulators, are combined in vitro, and reassembled via PCR to generate full length recombinant nucleic acids encoding transcription regulators. Alternatively, in vivo, in silico or other recombination methods are employed, as described herein.

The resulting library of nucleic acid variants is introduced into a population of host cells, e.g., E. coli or B. subtilis, under appropriate regulatory control, e.g., a constitutive or inducible promoter of a bacterial expression vector, e.g., pET3 series vectors, Stratagene, La Jolla, Calif.). Individual or pooled library members are transformed into host cells having a luciferase reporter under the control of a responsive promoter region, e.g., an aromatic catabolism operon cis regulatory region. Replicate subcultures are grown in the presence of small organic molecules of interest, and the subsultures screened for luciferase activity to identify recombinant (i.e., chimeric) transcriptional regulators with desired small organic molecule binding characteristics, e.g., specificity, affinity, etc.

### Example 2

### Transcription Regulator Array

The library members possessing desirable binding activities are recovered and the bacterial strains preserved in the presence of glycerol and frozen. Individual transformants are arrayed in a gridded matrix, and each transformant is assigned a unique identifier. If desired, information regarding the content and identification of library member pools is deconvoluted and the member components apportioned prior to establishing the array. Alternatively, the transformed host cells are cloned or pooled without screening and arranged in a stable array for storage and assay.

The gridded library members are accessed and cultures established for subsequent assay. For example, the gridded frozen cultures are accessed manually, or with robotic assistance, and new cultures are established preserving the information content of the array, e.g., in microtiter plates, for assay, e.g., by the luciferase reporter assay described above.

Alternatively, protein expression products are recovered from the identified transformants and arrayed on a responsive matrix as described above, e.g., a photoelectric chip sensitive to conformational changes induced by binding of the transcriptional regulator to a ligand.

### Example 3

### Detection of Stimulus Compounds

Regardless of the format of the library array, calibration and standardization is performed by exposing the array components to one or more known standard, e.g., calibrating or pattern forming, stimulus. For example, to standardize and calibrate the array for detection of small organic molecules, the array is contacted with known organic molecules, e.g., phenol, toluene, xylenol, and selected derivatives. The resulting response, e.g., luciferase or GFP activity, or “calibrating” array pattern, is detected and recorded, for example, by a CCD camera or other photoelectric device. The array is then exposed to one or more test stimulus. In the case of cultures, this can be accomplished by exposing replicate cultures to one or more test compounds, while in the case of proteins arrayed on a chip, this is best accomplished by washing under conditions amenable to preservation of the array, followed by subsequent exposure to the test compounds.

Alternative formats for performing detection assays, e.g., on microfluidic devices (e.g., LabMicrofluidic device® high throughput screening system (HTS) by Caliper Technologies, Mountain View, Calif. or the HP/Agilent technologies Bioanalyzer using LabChip™ technology by Caliper Technologies Corp. See, also, www.calipertech.com) are available and favorably employed in the context of the present invention.

### Example 4

### Diversification of Substrate Binding Properties

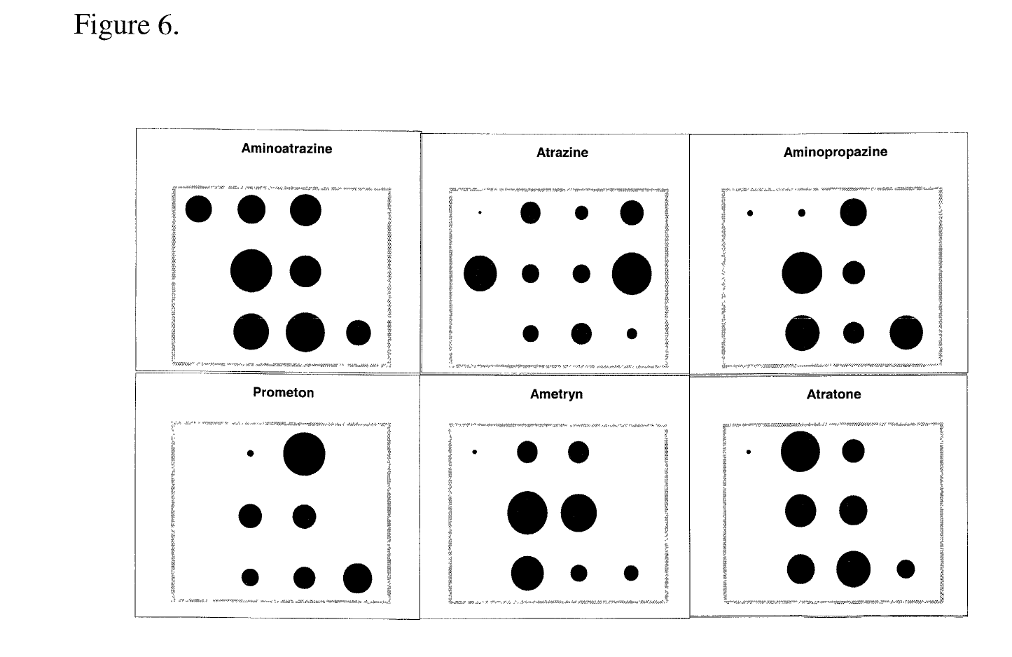

A set of related enzymes that recognize a diversity of substrates can be produced by diversification, by such procedures as DNA shuffling, of one or more parental enzymes. Approaches involving a single parental enzyme involve first mutagenizing the nucleic encoding the parental enzyme, e.g., by use of error prone amplification, e.g., error prone polymerase chain reaction (PCR). Two closely related triazine hydrolase enzymes were shuffled, resulting in a large set of enzymes with differing substrate specifcities, including activities towards five substrates that were not hydrolyzed by either of the parental enzymes. FIG. 6 shows the activities of an array of twelve of the triazine hydrolase homologue proteins towards six different, but chemically extremely similar, substrates (aminoatrazine, atrazine, aminopropazine, prometon, ametryn and atratone). The area of the circles are proportional to the activities of the enzyme towards the substrate. Three of the enzymes recognize only atrazine. Such enzymes are good candidates for single-analyte biosensors specific for atrazine when coupled to a signal transduction platform (as described above). In this example, however, none of the other enzyme variants could be used to uniquely identify any of the other substrate compounds: i.e., the enzyme variants have overlapping substrate specificities. Nonetheless, it is clear from FIG. 6, that the twelve enzymes have a different fingerprint of activities depending upon which compound present. Thus, the compounds can be identified based on the activities of the set of enzymes rather than the acitivity of any single enzyme. While the above example is relatively simple analytically, more complex samples can be deconvoluted using a (typically) computer assisted bioinformatics approach (as described above). Further details are provided, e.g., in “Triazine Degrading Enzymes,” PCT/US01/06654, filed Feb. 28, 2001.

Thus, DNA shuffling or other directed evolution methods can be used to produce substrate binding specificities and catalytic diversity suitable for detection of a wide variety of analytes, such as small molecule analytes, including those analytes for which no naturally occuring binding or catalytic specificity exists.

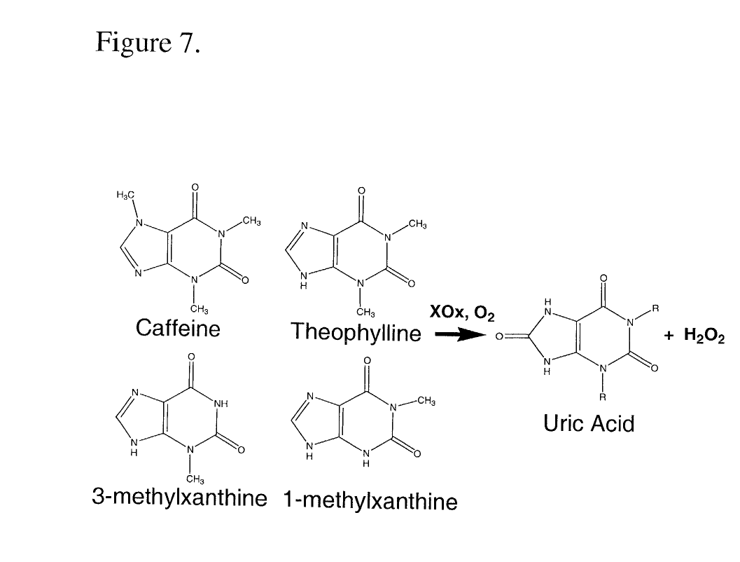

This general approach is useful for developing biosensors capable of detecting other classes of small molecules (e.g., with related structurs). For example, xanthine oxidase can be evolved to adapt it to an oxidase-based biosensor platform for the detection of the pharmaceutical drug theophylline. However, the enzyme is unable to differentiate between theophyllin and other metabolites with similar structures (as shown in FIG. 7). As described above with respect to atrazine, a set of enzymes (enzyme variants) can be produced by directed evolution, that differentiate between these compounds. As discussed above, activity data from a set of such enymes can be used, e.g., in the context of a multi-analyte array to determine which of the (one or more) compounds is present in a sample, such as a serum, blood or urine sample.

### Example 5

### Warfarin Biosensor

The Cytochrome P450 family is one of the largest and oldest superfamilies of enzymes known (see, e.g., dmelson.utmem.edu/CytochromeP450.html). It contains over 200 known families, thousands of sequences and several crystal structures. The superfamily is structurally and functionally well conserved but very diverse in sequence and substrate space (see, e.g., drnelson.utmem.edu/PIR.P450.description.html). Cytochrome P450 isozymes provide an example of a generic recognition element with a variety of substrate specificities, and a common mediator based electrochemical read out.

Cytochrome P450s are hemoproteins which catalyse an extremely large number of biological oxidations upon substrates as varied as steroids, polyketides, polyaromatics, fatty acids and many xenobiotics and drugs. In spite of the variation in substrates, the mechanism of catalysis is identical. The cytochrome P450 oxidation system consists of two components, the P450 itself, which is the catalytic moiety, and the electron transport chain. The electron transport chain differs between eukaryotes and prokaryotes but functions in a similar manner. In all cases, the first step of the catalytic cycle is substrate binding. This displaces an active site water and causes the iron to switch spin state. This changes the reduction potential of the heme, and at this point the electron transport proteins can transfer an electron to the iron in the heme group. Once the iron is reduced oxygen binds. After a few rearrangements, the active iron-oxo species is generated and oxidation of the substrate occurs. This system is designed to prevent the generation of the active oxidizing species in the absence of substrate and help prevents autoinactivation of the protein.

As a result of this mechanism, cytochrome P450s are an ideal family of proteins to directly connect with electronic systems. A cytochrome P450 directly deposited on the surface of an electrode gives a measurable change in the electrode, either by cyclic voltametry or current flow. Another method is to use the P450 as the gate electrode of a field effect transistor (BioFET). FETs are readily manufactured in dense arrays and are modulated by a change in the electrical potential at the gate electrode. ChemFETs work due to a pHlion change in a polymer on the surface of the FET whereas we would electrically contact the haem group to the electrode. Examples of simpler devices are described, e.g., in Brand et al. (1991) Appl Microbiol Biotechnol 36:167-172.

Several features of the Cytochrome P450 family make it ideally suited for biosensor applications. Firstly, the entire family of enzymes has similar redox potentials making it possible to employ a single mediator, even across a multi-analyte array. Secondly, all of the enzymes perform the same chemistry. It is known that you can take the active site of a cytochrome P450 with specificity X, and graft it onto the catalytic domain of P450 Y, to get a cytochrome P450 protein with specificity X. Further details are provided in, e.g., WO 00/09682, published Feb. 24, 2000. However, essentially any redox active protein is amenable to the approach herein described.

In addition to the properties and features described above, the greatest single class of mediators of drug metabolism in humans are cytochrome P450s, see, e.g., www.georgetown.edu/departments/pharmacology/davetab.html. The specificities of these isozymes are well described and include most compounds of pharmaceutical importance.

Cytochrome P450s are capable of oxidizing unactivated C-H bonds. Therefore, essentially any substrate analyte that binds well can be measured. In general, P450 ligands are hydrophobic (e.g., steroids, terpenes, alkanes, fatty acids etc) but this property is not exclusive (e.g., ethanol, erythromycin precursor, etc.). Furthermore, Flavin dependent oxidases tend to oxidize hydrophilic substrates (amino acids, sugars etc), and are also suitable for the strategy described herein. Thus, the two families provide adaptable binding specificities for most of the compounds of interest for sensing applications.

Finally, bacterial P450s are soluble, readily expressed and recovered proteins that are typically produced at greater that 10 mg/L of protein in E coli. In addition, the proteins are easy to produce and screen in vivo (red/brown colonies).

Cytochrome P450 isozyme variants are used to produce a biosensor for the cardiac drug Warfarin, in the following example. Warfarin is a very effective therapeutic agent for the control of angina. Systemic administration of Warfarin reduces the viscosity of blood, i.e., it “thins” the blood, reducing the symptoms of angina. However, Warfarin has a very narrow therapeutic range and significant potential toxicity which limits its use. A biosensor for home or clinical use is of significant value, enabling a patient to control the concentration of Warfarin in the body, reducing potential side effects, and increasing applicability of the drug as well as its efficacy.

Warfarin is a coumarin derivative with which no obvious flavin oxidase activity is associated in the literature. However, the interactions with cytochrome P450 have been well described. In vivo, Warfarin is oxidized by cytochrome P450 2C9, which is one of the major drug metabolizing isozymes described to date, see, e.g., www.georgetown.edu/departments/pharmacology/davetab.html. It is also oxidized by bacterial cytochrome P450 isozyme 105 D1. The latter enzyme has several closely related homologues in the database (drnelson.utmem.edu/bacteria.2000.html) many more should be accessible using well-known techniques. The domain structure of this protein has also been described (www.expasv.ch/cgi-bin/get-sprot-entry P26911, http://p450.abc.hu/P450domains.html). P450 105D1 is derived from the bacterial species S. griseus and has a molecular weight of ˜40 kDa. This isozyme has been expressed in E. coli at ˜12 mg/L and has been shown to be active (if at reduced levels) after immobilization to DE 52 resin. (see, e.g., BBRC., 279, 708-711, 2000).

Following isolation of one or more homologues, the parental sequences are diversified, e.g., by shuffling or other procedures, to generate a diverse sequence library encoding cytochrome P450 variants. An initial screen for P450 activity can be performed by induction on a solid surface (agar or nylon) followed by detection of colonies that have become brown due to P450 induced Haem synthesis and incorporation. Reduction and carbon monoxide treatment enables the detection of productively folded P450s on the surface of the agar or membrane.

Purification and immobilization of the active proteins on an electrode array can be accomplished by any of the means described above, e.g., with respect to a glucose oxidase based sensor, with the exception that different redox mediator may be required or desirable. Alternative redox mediators are known in the art, and of skill in the art is able to empirically determine which candidate redox mediator is suitable for a particular application. It should be noted that the redox potential for P450 isozymes (see, e.g., www.uky.edu/Pharmacy/ps/porter/CPR enzymology.htm) typically drops by ˜100 mV on substrate binding to ˜−270mV and in most cases the electrons are provided to the P450 isozymes by reduced flavins.

Following assembly of the library, or a selected or random subset of the library into an array, the array is not only adjusted for activity towards Warfarin, but also to a large number of other molecules with different chemical structures, providing data useful in generating molecular signatures or fingerprints, and subsequently for the generation of algorithms associating the fingerprints with analyte identity.

Upon identification of a single enzyme variant that binds Warfarin with high selectivity, a single response element can then be produced analogous to the glucose sensor described above. Arrays of less selective, or less sensitive enzymes can be, nonetheless, utilized as an array, for example, as an array with predictive value in predicting drug metabolism.

The expected structure space covered by this initial library should overlap with the specificity of cytochrome 2C9. Further libraries that covered the structure space of the other important human P450s (www.georgetown.edu/departments/pharmacology/davetab.html) would then be constructed and finally an array of arrays would give the full spectrum of P450 specificities seen in man.

One significant barrier to the formation of an array, such as those described above, is the intractability of the protein to handling. For example, eukaryotic proteins are membrane associated. The methods described herein can be used to diversify and select a family of Cytochrome P450s, either whole or a truncated form, for stability and activity in an immobilized array. The following properties are selected, sequentially or simultaneously, from among members of the diversified library. Initially, the activity of the immobilized proteins will be assessed by the ability to form a CO difference spectra, an activity which directly measures the spin state change, but not substrate binding, and by use of turnover with the peroxide shunt, which measures productive substrate binding. Finally, binding is assessed on the surface of an electrode, enabling the production of appropriate signal processing software and hardware.

For example, in one approach, the surface of an electrode is coated with a Nickel-NTA (Ni-NTA) mixture, or other small molecule binding motif. and a masked permanent attachment site.

The biosensor protein or library of biosensor proteins are then expressed as fusion proteins including a Histidine tag (or other domain corresponding to the small molecule binding motif) and the cells lysed. The cell lysate is then spotted onto a masked surface to which the Ni-NTA is adhered under conditions where the His Tag binds to the Ni-NTA. The non-specifically bound proteins are then washed off the surface. In order to prevent the multiple proteins in an array from cross-contaminating, spots on the surface corresponding to individual members of the library can be demarcated by a hydrophobic surface. If a larger surface volume for binding is required, then the entire process can be performed in an etched pit on the surface or other three dimensional format.

Once the protein or proteins have been purified and attached to the surface, they are covalently attached. This is achieved by unmasking permanent attachment sites, e.g., a surface covered with diol moieties. Once the protein of interest was held at the surface by the interaction between the His tag moiety of the protein and the Ni-NTA on the surface, a solution of periodate is washed on. The vicinal diols are cleaved to form an aldehyde, which forms a Schiff's base with the surface amines of the protein. A second wash with sodium cyanoborohydride permanently affixes the protein to the surface. Other chemistries are easily designed, such as alkenes that are osmium tetroxide/NaIO₄ treated to form an aldehyde. Alternatively a masked thiol is used followed by a bifunctional S-N coupling reagent. Even a masked amine can be unmasked followed by a glutaraldehyde treatment. A number of similar chemical compounds are commercially available, e.g., from Pierce (Rockford, Ill.) and Molecular Probes (Eugene, Oreg.) sell many similar components. In cases where electrical connection to the surface is desired, a conjugating system, such as an activated thiophene, can be used to attach the protein(s) to the surface.

This two stage attachment protocol offers a number of advantages. Firstly, this procedures enables affinity purification of the protein, or library of proteins, from a complex mixture. Secondly, only after the protein is substantially pure does final attachment to the surface take place. Finally, this method avoids purification of the proteins prior to attachment to the surface.

This method also facilitates optimization of proteins for use in a biosensor by providing a simple, cost-effective, immbolization method and format suitable for screening variants for desired properties. Using this method, a library of diversified proteins can be analysed for their catalytic (or other) characteristics in an environment much closer to the desired working format.

The cytochrome P450 superfamily, has two primary functions in nature. A first subset of P450 family members are involved in catabolism, these are extremely specific for their intended substrates, e.g., steroids and polyketides. Such P450 isozymes make excellent specific detectors for the molecules in question. The other main class of P450s is involved in the hydroxylation of molecules for xenobiotic detoxification or use as a carbon source. These enzymes each recognize broad classes of substrates, such as polyaromatics or tertiary amines, and are ideal for mapping the broad profile of compounds present in a sample. An array of the naturally occurring P450s, thus, provides both specific and general information about the analytes in a sample. Pattern recognition software, as described herein, is used to identify various analytes by the differential response, i.e., “fingerprint,” across the array.

Table 3: Exemplary Targets, Relevant Enzymes, and Detection Schemes

The biosensors and biosensor arrays of the invention can be used to detect a wide variety of analytes, especially small molecule analytes relevant to quantitating, monitoring, characterizing or otherwise assessing varied environmental and medical samples for the presence of, e.g., herbicides, blood gases, blood electrolytes, environmental contaminants, soil composition, water solutes and particulates, air, food, toxins, HCl, ozone, alcohol, sugars, pathogens, chemical and biological warfare agents, etc.

The following table provides various targets, relevant enzymes or proteins, detection schemes, and the like. This table is only exemplary—many additional features are set forth above, and the table should not be considered limiting, in any way.

| Targets | Enzymes or Ab | Detection | Comments |
|---|---|---|---|
| Metals (Ag+, Hg2+, Pb2+, | Urease (inhibition) | electrochem, ammonium | |
| Cd2+, Zn2+, Fe3+, Cr3+, | Glucose oxidase | electrochem | 1-100 uM sensitivity |
| | Alcohol oxidase | electrochem | 1-100 uM sensitivity |
| | Butyril oxidase | electrochem | 1-100 uM sensitivity |
| | Proteases | optical | |
| Pesticides | Acetyl choline esterase | choline esterase | nM sensitivity |
| | (inhibition) | Clark electrode | |
| | | pH | |
| Organophosphorous | Organophosphate | Clark electrode | |
| | hydrolase | | |
| Carbamates | Cholineestrases | pH | ng/L sensitivity |
| Atrazine | Ab | SPR, piezoelectric | 0.1 ppb sens |
| Nitrate | Nitrate reductase | electrochemical | |
| Ammonia | Glutamate | electrochemical | |
| | dehydrogenase | | |
| Phenols | Polyphenol oxidase | clark electrode | Product of several ag and chem |
| | Tyrosinase | \other electrochemical | processes. |
| Nitric Oxide Metabolites | Nitrous oxide reductase | electrochemical | |
| Formaldehyde | Formaldehyde | piezoelectric | 1-100 ppb sens, very selective |
| | dehydrogenase | | |
| Parathioon | Ab | | |
| Sucrose | Invertase | thermometric | stability probs |
| Cocaine | Ab | piezoelectric | 0.1 ng sensitivity |
| Cholesterol | Cholesterol esterase | | stability probs |
| Organophosphorous nerve | cholinesterase | piezoelectric | ppb sens |
| agents | | | |
| Glucose | glucose oxidase | clark electrode | Electrochemical |
| Lactate | lactate dehydrogenase | | |
| Urea | urease | | |
| Creatinine | | electrochem, ammonium | |
| Adrenaline | Ab | ELISA | |
| Dopamine | Ab | ELISA | |
| Histamine | Ab | ELISA | |
| Melatonin | Ab | ELISA | |
| Metanephrine | Ab | ELISA | |
| Serotonin | Ab | ELISA | |

TABLE 4
HORMONES SUITABLE FOR POTENTIAL BIOSENSOR
DEVELOPMENT.
TABLE OF HUMAN HORMONES

| Organ | Hormone | Structure | Function |
|---|---|---|---|
| Pituitary- | Thyroid-stimulating | Protein | Stimulates the thyroid gland to secrete its |
| anterior | hormone (TSH) | | hormones |
| All are released in | Follicle-stimulating | Protein | In females - Stimulates follicles to release |
| response to the | hormone (FSH) | | estrogens |
| secretion of | | | In males - Helps spermatogonia produce sperm |
| certain hormones | Luteinizing hormone | Protein | In females - Stimulates the follicles to release |
| from the | (LH) | | estrogens, triggers completion of meiosis I of |
| hypothalamus | | | egg, stimulates empty follicle to develop into |
| | | | corpeus luteum, which secretes progesterone in |
| | | | latter half of menstrual cycle |
| | | | In males - Stimulates testes to secrete |
| | | | testosterone |
| | Prolactin (PRL) | Protein | In females - Prepares breasts for milk |
| | | | production |
| | Growth hormone (GH) | Protein | Stimulates liver to release IGF-I, which |
| | | | promotes growth of long bones |
| | Adrenocorticotropic | Peptide | Stimulates adrenal cortex to produce |
| | hormone (ACTH) | | glucocorticoids, mineralocorticoids, androgens. |
| Pituitary- | Antidiuretic hormone | Peptide | Acts on collecting ducts of kidneys to facilitate |
| posterior | (ADH) | | reabsorption of water into blood |
| All are released in | Oxytocin | Peptide | Stimulates contractions of uterus at time of birth, |
| response to the | | | stimulates release of milk when baby suckles |
| secretion of | | | |
| certain hormones | | | |
| from the | | | |
| hypothalamus | | | |
| Hypothalamus | Thyrotropin-releasing | Peptide | Stimulates release of TSH and PRL |
| | hormone (TRH) | | |
| All are released | Gonadotropin- | Peptide | Increases release of FSH and LH |
| into the blood in | releasing hormone | | |
| periodic spurts | (GHRH) | | |
| and travel to the | Corticotropin- | Peptide | Stimulates release of GH |
| anterior lobe of | releasing hormone | | |
| the pituitary | (CRH) | | |
| | Somatostatin | Peptide | Inhibits release of GH and TSH |
| | Dopamine | Tyrosine | Inhibits release of PRL |
| | | derivative | |
| Pineal gland | Melatonin | Tryptophan | Regulates circadian cycles (sleep/awake |
| | | derivative | patterns) |
| Thyroid gland | Thyroxine (T4) | Tyrosine | Regulates metabolic rate (as measured by |
| | | derivative | oxygen uptake) and heart rate |
| | Triiodothyronine (T3) | Tyrosine | Regulates metabolic rate (as measured by |
| | | derivative | oxygen uptake) and heart rate |
| | Calcitonin | Peptide | Promotes transfer of Ca to bones |
| Parathyroid | Parathyroid hormone | Protein | Increases concentration of Ca to blood |
| glands | (PTH) | | |
| Adrenal cortex | Glucocorticoids | Steroids | level of glucose in blood |
| All are released in | Mineralocorticoids | Steroids | Promotes reabsorption of salt into blood to |
| response to | | | maintain normal blood pressure |
| secretion of | Androgens | Steroids | Promotes masculinization |
| ACTH from | | | |
| anterior lobe of | | | |
| pituitary | | | |
| Adrenal medulla | Adrenaline | Tyrosine | Increased heartrate, blood pressure, metabolic |
| | (epinephrine) | derivative | rate, blood sugar, dilation of bronchi and pupils, |
| Comprised of | Noradrenaline | Tyrosine | reduced clotting time for blood |
| neurons so also | (norepinephrine) | derivative | |
| part of CNS | | | |
| Ovarian follicle | Estrogens | Steroid | Contribute to development of breasts, uterus, |
| | | | vagina, broadening of pelvis, increase in fat |
| | | | tissue, minimize loss of Ca from bone, promote |
| | | | blood clotting |
| Corpus luteum | Progesterone | Steroid | Prepares endometrium for pregnancy, inhibits |
| and placenta | | | contraction of uterus, inhibits development of |
| | | | new follicles, inhibits FSH and LH |
| Trophoblast and | Human chorionic | Protein | Prevents deterioration of corpus luteum, |
| placenta | gonadotropin (HCG) | | promotes continuation of pregnancy |
| Testes | Androgens | Steroid | Promotes development of secondary sexual |
| | | | characteristics of men, essential for sperm |
| | | | production |
| Pancreas (Islets of | Insulin | Protein | Stimulates liver cells to take up glucose from |
| Langerhans) | | | blood and convert it to glycogen, stimulates |
| | | | synthesis of fat, results in drop in blood sugar |
| | Glucagon | Peptide | Stimulates conversion of glycogen to glucose, |
| | | | helps maintain steady blood sugar |
| | Somatostatin | Peptide | Reduces rate at which food is absorbed by |
| | | | intestine |
| Kidney | Renin | Protein | Increases blood pressure |
| | Erythropoietin (EPO) | Protein | Increases production of red blood cells |
| | Calcitrol | Steroid | Promotes absorption of calcium into intestine |
| | | derivative | |
| Skin | Calciferol (vitamin | Steroid | Classified as a hormone (as well as a vitamin) |
| | D3) | derivative | because it is made in certain cells, carried in the |
| | | | blood and affects gene transcription in target |
| | | | cells |
| Heart | Atrial-natriuretic | Peptide | Lowers blood pressure by relaxing arterioles, |
| | peptide (ANP) | | inhibiting secretion of rennin and aldosterone, |
| | | | and inhibiting reabsorption of Na by kidneys |
| Stomach and | Gastrin | Peptide | Stimulates exocrine cells of stomach to secrete |
| intestines | | | gastric juice (HCl and pepsin) |
| | Secretin | Peptide | Stimulates exocrine part of pancreas to secrete |
| | | | bicarbonate to neutralize acidity of stomach |
| | | | contents |
| | Cholecystokinin | Peptide | Stimulates gall bladder to release bile, stimulates |
| | (CCK) | | pancreas to release pancreatic digestive enzymes |
| | | | into pancreatic fluid - results in inhibition of |
| | | | gastrin, secretin, CCK, glucagon |
| | Somatostatin | Peptide | Reduces rate at which food is absorbed by |
| | | | intestine |
| | Neuropeptide Y | Peptide | Causes increased storage of ingested food as fat |
| Liver | Insulin-like growth | Protein | Stimulates growth of long bones |
| | factor (IGF) | | |
| | Angiotensinogen | Protein | Precursor of angiotensinogen, which is split by |
| | | | renin resulting in an increase blood pressure |
| | Thrombopoietin | Protein | Stimulates blood clotting |
| Fat cells | Leptin | Protein | Inhibits food intake |

In a further aspect, the present invention provides for the use of any apparatus, apparatus component, composition or kit herein, for the practice of any method or assay herein, and/or for the use of any apparatus or kit to practice any assay or method herein. In one incarnation, such an array could be utilized as a test-kit for libraries of biopolymers. Any point on the array that responded to the stimulus would correspond to a series of proteins in the whole library. This starting point could then be further optimized for the desired property.

TABLE 5
EXEMPLARY OXIDOREDUCTASE ENZYMES

| | | |
|---|---|---|
| | EC 1.1.1.34 hydroxymethylglutaryl-CoA reductase | EC 1.1.1.67 mannitol 2-dehydrogenase |
| EC 1.1.1 | (NADPH₂) | EC 1.1 1.68 now EC 1.7.99.5 |
| | EC 1.1.1.35 3-hydroxyacyl-CoA dehydrogenase | EC 1.1.1.69 gluconate 5-dehydrogenase |
| WITH AND OR NADP AS ACCEPTOR | EC 1.1.1.36 acetoacetyl-CoA reductase | EC 1.1.1.70 deleted, included in EC 1.2.1.3 |
| | EC 1.1.1.37 malate dehydrogenase | EC 1.1.1.71 alcohol dehydrogenase [AND(P)] |
| EC 1.1.1.1 alcohol dehydrogenase | EC 1.1.1.38 malate dehydrogenase (oxaloacetate- | EC 1.1.1.72 glycerol dehydrogenase (NADP) |
| EC 1.1.1.2 alcohol dehydrogenase (NADP) | decarboxylating) | EC 1.1 1.73 octanol dehydrogenase |
| EC 1.1.1.3 homoserine dehydrogenase | EC 1.1.1.39 malate dehydrogenase (decarboxylating) | EC 1.1.1.74 deleted |
| EC 1.1.1.4 (R,R)-butanediol dehydrogenase | EC 1.1.1.40 malate dehydrogenase (oxaloacetate- | EC 1.1.1.75 (R)-aminopropanal dehydrogenase |
| EC 1.1.1.5 acetoin dehydrogenase | decarboxylating) (NADP) | EC 1.1.1.76 (S,S)-butanediol dehydrogenase |
| EC 1.1.1.6 glycerol dehydrogenase | EC 1.1.1.41 isocitrate dehydrogenase (AND) | EC 1.1.1.77 lactaldehyde reductase |
| EC 1.1.1.7 propanediol-phosphate dehydrogenase | EC 1.1.1.42 isocitrate dehydrogenase (NADP | EC 1.1 1.78 D-lactaldehyde dehydrogenase |
| EC 1.1.1.8 glycerol-3-phosphate dehydrogenase (AND) | EC 1.1.1.43 phosphogluconate 2-dehydrogenase | EC 1.1.1.79 glyoxylate reductase (NADP) |
| EC 1.1.1.9 D-xylulose reductase | EC 1.1.1.44 phosphogluconate dehydrogenase | EC 1.1.1.80 isopropanol dehydrogenase (NADP) |
| EC 1.1.1.10 L-xylulose reductase | (decarboxylating) | EC 1.1.1.81 hydroxypyruvate reductase |
| EC 1.1.1.11 D-arabinitol 4-dehydrogenase | EC 1.1.1.45 L-gulonate 3-dehydrogenase | EC 1.1.1.82 malate dehydrogenase (NADP) |
| EC 1.1.1.12 L-arabinitol 4-dehydrogenase | EC 1.1.1.46 L-arabinose 1-dehydrogenase | EC 1.1 1.83 D-malate dehydrogenase |
| EC 1.1.1.13 L-arabinitol 2-dehydrogenase | EC 1.1.1.47 glucose 1-dehydrogenase | (decarboxylating) |
| EC 1.1.1.14 L-iditol 2-dehydrogenase | EC 1.1.1.48 galactose 1-dehydrogenase | EC 1.1.1.84 dimethylmalate dehydrogenase |
| EC 1.1.1.15 D-iditol 2-dehydrogenase | EC 1.1.1.49 glucose-6-phosphate 1-dehydrogenase | EC 1.1.1.85 3-isopropylmalate dehydrogenase |
| EC 1.1.1.16 galactitol 2-dehydrogenase | EC 1.1.1.50 3α-hydroxysteroid dehydrogenase (B- | EC 1.1.1.86 ketol-acid reductoisomerase |
| EC 1.1 1.17 mannitol-1-phosphate 5-dehydrogenase | specific) | EC 1.1.1.87 3-carboxy-2-hydroxyadipate |
| EC 1.1.1.18 inositol 2-dehydrogenase | EC 1.1.1.51 3(or 17)β-hydroxysteroid dehydrogenase | dehydrogenase |
| EC 1.1.1.19 L-glucuronate reductase | EC 1.1.1.52 3α-hydroxycholanate dehydrogenase | EC 1.1.1.88 hydroxymethlglutaryl-CoA reductase |
| EC 1.1.1.20 glucuronolactone reductase | EC 1.1.1.53 3α(or 20β)-hydroxysteroid dehydrogenase | EC 1.1.1.89 deleted, included in EC 1.1.1.86 |
| EC 1.1.1.21 aldehyde reductase | EC 1.1.1.54 allyl-alcohol dehydrogenase | EC 1.1.1.90 aryl-alcohol dehydrogenase |
| EC 1.1.1.22 UDPglucose 6-dehydrogenase | EC 1.1.1.55 lactaldehyde reductase (NADPH₂) | EC 1.1.1.91 aryl-alcohol dehydrogenase (NADP) |
| EC 1.1.1.23 histidinol dehydrogenase | EC 1.1.1.56 ribitol 2-dehydrogenase | EC 1.1.1.92 oxaloglycolate reductase |
| EC 1.1.1.24 quinate 5-dehydrogenase | EC 1.1.1.57 fructuronate reductase | (decarboxylating) |
| EC 1.1.1.25 shikimate 5-dehydrogenase | EC 1.1.1.58 tagaturonate reductase | EC 1.1.1.93 tartrate dehydrogenase |
| EC 1.1.1.26 glyoxylate reductase | EC 1.1.1.59 3-hydroxypropionate dehydrogenase | EC 1.1.1.94 glycerol-3-phosphate dehydrogenase |
| EC 1.1.1.27 L-lactate dehydrogenase | EC 1 1.1.60 2-hydroxy-3-oxopropionate reductase | [AND(P)] |
| EC 1.1.1.28 D-lactate dehydrogenase | EC 1.1.1.61 4-hydroxybutyrate dehydrogenase | EC 1.1 1.95 phophoglycerate dehydrogenase |
| EC 1.1.1.29 glycerate dehydrogenase | EC 1.1.1.62 estradiol 17β-dehydrogenase | EC 1.1.1.96 diiodophenylpyruvate reductase |
| EC 1.1.1.30 3-hydroxybutyrate dehydrogenase | EC 1.1.1.63 testosterone 17β-dehydrogenase | EC 1.1.1.97 3-hydroxybenzyl-alcohol dehydrogenase |
| EC 1.1.1.31 3-hydroxyisobutyrate dehydrogenase | EC 1.1.1.64 testosterone 17β-dehydrogenase (NADP) | EC 1.1.1.98 (R)-2-hydroxy-fatty-acid dehydrogenase |
| EC 1.1.1.32 mevaldate reductase | EC 1.1.1.65 pyridoxine 4-dehydrogenase | EC 1.1.1.99 (S)-2-hydroxy-fatty-acid dehydrogenase |
| EC 1.1.1.33 mevaldate reductase (NADPH₂) | EC 1.1.1.66 ω-hydroxydecanoate dehydrogenase | EC 1.1 1.100 3-oxoacyl-[acyl-carrier-protein] |
| reductase | EC 1.1.1.140 sorbitol-6-phosphate 2-dehydrogenase | EC 1.1.1.176 12α-hydroxysteroid dehydrogenase |
| EC 1.1 1.101 acylglycerone-phosphate reductase | EC 1.1.1.141 15-hydroxyprostaglandin dehydrogenase | EC 1.1.1.177 glycerol-3-phosphate 1-dehydrogenase |
| EC 1.1.1.102 3-dehydrosphinganine reductase | (AND) | (NADP) |
| EC 1 1.1.103 L-threonine 3-dehydrogenase | EC 1.1.1.142 D-pinitol dehydrogenase | EC 1.1.1.178 3-hydroxy-2-2methylbutyryl-CoA |
| EC 1.1.1.104 4-oxoproline reductase | EC 1.1.1.143 sequoyitol dehydrogenase | dehydrogenase |
| EC 1.1.1.105 retinol dehydrogenase | EC 1.1.1.144 perillyl-alcohol dehydrogenase | EC 1.1.1.179 D-xylose 1-dehydrogenase (NADP) |
| EC 1.1.1.106 pantoate 4-dehydrogenase | EC 1.1.1.145 3β-hydroxy.Δ⁵-steroid dehydrogenase | EC 1.1.1.180 deleted, included in EC 1.1.1.131 |
| EC 1.1.1.107 pyridoxal 4-dehydrogenase | EC 1.1.1.146 11β-hydroxysteroid dehydrogenase | EC 1.1.1.181 cholest-5-ene-3β, 7α-diol 3β- |
| EC 1.1.1.108 carnitine 3-dehydrogenase | EC 1.1.1.147 16α-hydroxysteroid dehydrogenase | dehydrogenase |
| EC 1.1 1.109 now EC 1.3.1.28 | EC 1.1.1.148 estradiol 17α-dehydrogenase | EC 1.1.1.182 deleted, included in EC 1.1.1.198, EC |
| EC 1.1.1.110 indolelactate dehydrogenase | EC 1.1.1.149 20α-hydroxysteroid dehydrogenase | 1.1.1.227 and EC 1.1.1.228 |
| EC 1.1.1.111 3-(imidazol-5-yl)lactate dehydrogenase | EC 1.1.1.150 21-hydroxysteroid dehydrogenase (AND) | EC 1.1.1.183 geraniol dehydrogenase |
| EC 1.1.1.112 indanol dehydrogenase | EC 1.1.1.151 21-hydroxysteroid dehydrogenase | EC 1.1.1.184 carbonyl reductase (NADPH₂) |
| EC 1.1.1.113 L-xylose 1-dehydrogenase | (NADP) | EC 1.1.1.185 L-glycol dehydrogenase |
| EC 1.1.1.114 apiose 1-reductase | EC 1.1.1.152 3α-hydroxy-5β-androstane-17-one 3α- | EC 1.1.1.186 dTDPgalactose 6-dehydrogenase |
| EC 1.1.1.115 ribose 1-dehydrogenase (NADP) | dehydrogenase | EC 1.1 1.187 GDP-4-dehydro-D-rhamnose reductase |
| EC 1.1.1.116 D-arabinose 1-dehydrogenase | EC 1.1.1.153 sepiapterin reductase | EC 1.1.1.188 prostaglandin-F synthase |
| EC 1.1.1.117 D-arabinose 1-dehydrogenase [AND(P)] | EC 1.1.1.154 ureidoglycolate dehydrogenase | EC 1.1.1.189 prostaglandin-E₂ 9-reductase |
| EC 1 1.1 118 glucose 1-dehydrogenase (AND) | EC 1.1.1.155 homoisocitrate dehydrogenase | EC 1.1.1.190 indole-3-acetaldehyde reductase |
| EC 1.1.1.119 glucose 1-dehydrogenase (NADP) | EC 1.1.1.156 glycerol 2-dehydrogenase (NADP) | (NADH₂) |
| EC 1.1.1.120 galactose 1-dehydrogenase (NADP) | EC 1.1.1.157 3-hydroxybutyryl-CoA dehydrogenase | EC 1.1.1.191 indole-3-acetaldehyde reductase |
| EC 1.1.1.121 aldose 1-dehydrogenase | EC 1.1.1.158 UDP-N-acetylmuramate dehydrogenase | (NADH₂) |
| EC 1.1.1.122 D-threo-aldose 1-dehydrogenase | EC 1.1.1.159 7α-hydroxysteroid dehydrogenase | EC 1.1.1.192 long-chain-alcohol dehydrogenase |
| EC 1.1.1.123 sorbose 5-dehydrogenase (NADP) | EC 1.1.1.160 dihydrobunolol dehydrogenase | EC 1.1.1.193 5-amino-6-(5- |
| EC 1.1.1.124 fructose 5-dehydrogenase (NADP) | EC 1.1.1.161 cholestanetetraol 26-dehydrogenase | phosphoribosylamino)uracil reductase |
| EC 1.1 1.125 2-deoxy-D-gluconate 3-dehydrogenase | EC 1.1.1.162 erythrulose reductase | EC 1.1.1.194 coniferyl-alcohol dehydrogenase |
| EC 1.1.1.126 2-dehydro-3-deoxy-D-gluconate 6- | EC 1.1.1.163 cyclopentanol dehydrogenase | EC 1.1.1.195 cinnamyl-alcohol dehydrogenase |
| dehydrogenase | EC 1.1.1.164 hexadecanol dehydrogenase | EC 1.1.1.196 15-hydroxyprostaglandin-D |
| EC 1.1.1.127 2-dehydro-3-deoxy-D-gluconate 5- | EC 1.1.1 165 2-alkyn-1-ol dehydrogenase | dehydrogenase (NADP) |
| dehydrogenase | EC 1.1.1.166 hydroxycyclohexanecarboxylate | EC 1.1.1.197 15-hydroxyprostaglandin dehydrogenase |
| EC 1.1.1.128 L-idonate 2-dehydrogenase | dehydrogenase | (NADP) |
| EC 1.1.1.129 L-threonate 3-dehydrogenase | EC 1.1.1.167 hydroxymalonate dehydrogenase | EC 1.1.1.198 (+)-borneol dehydrogenase |
| EC 1.1.1.130 3-dehydro-L-gulonate 2-dehydrogenase | EC 1.1.1.168 2-dehydropantolactone reductase (A- | EC 1.1.1.199 (S)-usnate reductase |
| EC 1.1 1.131 mannuronate reductase | specific) | EC 1.1.1.200 aldose-6-phosphate reductase (NADPH₂) |
| EC 1.1.1.132 GDPmannose 6-dehydrogenase | EC 1.1.1.169 2-dehydropantoate 2-reductase | EC 1.1.1.201 7β-hydroxysteroid dehydrogenase |
| EC 1.1.1.133 dTDP-4-dehydrorhamnose reductase | EC 1.1.1.170 3β-hydroxy-4α- | (NADP) |
| EC 1.1.1.134 dTDP-6-deoxy-L-talose 4-dehydrogenase | methylcholestenecarboxylate 3-dehydrogenase | EC 1.1.1.202 1,3-propanediol dehydrogenase |
| EC 1.1 1.135 GDP-6-deoxy-D-talose 4-dehydrogenase | (decarboxylating) | EC 1.1.1.203 uronate dehydrogenase |
| EC 1.1.1.136 UDP-N-acetylglucosamine 6- | EC 1.1.1.171 now EC 1.5.1.20 | EC 1.1.1.204 xanthine dehydrogenase |
| dehydrogenase | EC 1.1.1.172 2-oxoadipate reductase | EC 1.1.1.205 IMP dehydrogenase |
| EC 1.1.1.137 ribitol-5-phosphate 2-dehydrogenase | EC 1.1.1.173 L-rhamnose 1-dehydrogenase | EC 1.1.1.206 tropine dehydrogenase |
| EC 1.1.1.138 mannitol 2-dehydrogenase (NADP) | EC 1.1.1.174 cyclohexane-1,2-diol dehydrogenase | EC 1.1.1.207 (·)-menthol dehydrogenase |
| EC 1.1.1.139 deleted, included in EC 1.1.1.21 | EC 1.1.1.175 D-xylose 1-dehydrogenase | EC 1.1.1.208 (+)-neomenthol dehydrogenase |
| EC 1.1.1.209 3(or 17)α-hydroxysteroid dehydrogenase | (AND) | EC 1.1.2.5 D-lactate dehydrogenase (cytochrome c- |
| EC 1.1.1.210 3β(or 20α)-hydroxysteroid | EC 1.1.1 240 N-acetyLhexosamine 1-dehydrogenase | 553) |
| dehydrogenase | EC 1.1.1.241 6-endo-hydroxycineole dehydrogenase | |
| EC 1.1.1.211 long-chain-3-hydroxyacyl-CoA | EC 1.1.1.242 now EC 1.3.1.69 | |
| dehydrogenase | EC 1.1.1.243 carveol dehydrogenase | EC 1.1.3 |
| EC 1.1.1.212 3-oxoacyl-[acyl-carrier-protein] | EC 1.1.1.244 methanol dehydrogenase | |
| reductase (NADH₂) | EC 1.1.1.245 cyclohexanol dehydrogenase | WITH OXYGEN AS ACCEPTOR |
| EC 1.1.1.213 3α-hydroxysteroid dehydrogenase (A- | EC 1.1.1.246 pterocarpin synthase | |
| specific) | EC 1.1.1.247 codeinone reductase (NADPH) | EC 1.1.3.1 deleted, included in EC 1.1 3 15 |
| EC 1.1.1.214 2-dehydropantolactone reductase (B- | EC 1.1.1.248 salutaridine reductase (NADPH) | EC 1.1.3.2 now EC 1.13.12.4 |
| specific) | EC 1.1.1.249 reinstated as EC 2.5.1.46 | EC 1.1.3.3 malate oxidase |
| EC 1.1.1.215 gluconate 2-dehydrogenase | EC 1.1.1.250 D-arabinitol 2-dehydrogenase | EC 1.1.3.4 glucose oxidase |
| EC 1.1.1.216 farnesol dehydrogenase | EC 1.1.1.251 galactitol-1-phosphate 5-dehydrogenase | EC 1.1.3.5 hexose oxidase |
| EC 1.1.1.217 benzyl-2-methyl-hydroxybutyrate | EC 1.1.1.252 tetrahydroxynaphthalene reductase | EC 1.1.3.6 cholesterol oxidase |
| dehydrogenase | EC 1.1.1.253 pteridine reductase | EC 1.1.3.7 aryl-alcohol oxidase |
| EC 1.1.1.218 morphine 6-dehydrogenase | EC 1.1.1.254 (S).carnitine 3-dehydrogenase | EC 1.1.3.8 L-gulonolactone oxidase |
| EC 1.1 1.219 dihydrokaempferol 4-reductase | EC 1.1 1.255 mannitol dehydrogenase | EC 1.1.3.9 galactose oxidase |
| EC 1.1.1.220 6-pyruvoyltetrahydropterin 2′-reductase | EC 1.1.1.256 fluoren-9-ol dehydrogenase | EC 1.1.3.10 pyranose oxidase |
| EC 1.1.1.221 vomifoliol 4′-dehydrogenase | EC 1.1.1.257 4-(hydroxymethyl)benzenesulfonate | EC 1.1.3.11 L-sorbose oxidase |
| EC 1.1.1.222 (R)-4-hydroxyphenyllactate | dehydrogenase | EC 1.1.3.12 pyridoxine 4-oxidase |
| dehydrogenase | EC 1.1.1.258 6-hydroxyhexanoate dehydrogenase | EC 1.1.3.13 alcohol oxidase |
| EC 1.1.1.223 isopiperitenol dehydrogenase | EC 1.1.1.259 3.hydroxypimeloyl-CoA dehydrogenase | EC 1.1.3.14 catechol oxidase (dimerizing) |
| EC 1.1.1.224 mannose-6-phosphate 6-reductase | EC 1.1.1.260 sulcatone reductase | EC 1.1.3.15 (S)-2-hydroxy-acid oxidase |
| EC 1.1.1.225 chlordecone reductase | EC 1.1.1.261 glycerol-1-phosphate dehydrogenase | EC 1.1.3.16 ecdysone oxidase |
| EC 1.1.1.226 4-hydroxycyclohexanecarboxylate | [AND(P)] | EC 1.1.3.17 choline oxidase |
| dehydrogenase | EC 1.1.1.262 4-hydroxythreonine-4-phosphate | EC 1.1.3.18 secondary-alcohol oxidase |
| EC 1.1.1 227 (·)-borneol dehydrogenase | dehydrogenase | EC 1.1.3.19 4-hydroxymandelate oxidase |
| EC 1.1.1.228 (+)-sabinol dehydrogenase | EC 1.1.1.263 1,5-anhydro-D-fructose reductase | EC 1.1.3.20 long-chain-alcohol oxidase |
| EC 1.1.1 229 diethyl 2-methyl-3-oxosuccinate | EC 1.1.1.264 L-idonate 5-dehydrogenase | EC 1.1.3.21 glycerol-3-phosphate oxidase |
| reductase | EC 1.1.1.265 3-methylbutanal reductase | EC 1.1.3.22 xanthine oxidase |
| EC 1.1.1.230 3α-hydroxyglycyrrhetinate | EC 1.1.1.266 dTDP-4-dehydro-6-deoxyglucose | EC 1.1.3.23 thiamin oxidase |
| dehydrogenase | reductase | EC 1.1.3.24 L-galactonolactone oxidase |
| EC 1.1.1.231 15-hydroxyprostaglandin-l | | EC 1.1.3.25 cellobiose oxidase |
| dehydrogenase (NADP) | | EC 1.1.3.26 columbamine oxidase |
| EC 1.1.1.232 15-hydroxyicosatetraenoate | EC 1.1.2 | EC 1.1.3.27 hydroxyphytanate oxidase |
| dehydrogenase | | EC 1.1.3.28 nucleoside oxidase |
| EC 1.1.1.233 N-acylmannosamine 1-dehydrogenase | WITH A CYTOCHROME AS ACCEPTOR | EC 1.1.3.29 N-acylhexosamine oxidase |
| EC 1.1.1 234 flavanone 4-reductase | | EC 1.1.3.30 polyvinyl-alcohol oxidase |
| EC 1.1.1.235 8-oxocoformycin reductase | EC 1.1.2.1 now EC 1.1.99.5 | EC 1.1.3.31 methanol oxidase |
| EC 1.1.1.236 tropinone reductase | EC 1.1.2.2 mannitol dehydrogenase (cytochrome | EC 1.1.3.32 (S)-stylopine synthase |
| EC 1.1.1.237 hydroxyphenylpyruvate reductase | EC 1.1.2.3 L-lactate dehydrogenase (cytochrome | EC 1.1.3.33 S-cheilanthifoline synthase |
| EC 1.1.1.238 12β-hydroxysteroid dehydrogenase | EC 1.1.2.4 D-lactate dehydrogenase (cytochrome | EC 1.1.3.34 berbamunine synthase |
| EC 1.1.1.239 3α(17β)-hydroxysteroid dehydrogenase | | EC 1.1.3.35 salutaridine synthase |
| EC 1.1.3.36 (S).canadine synthase | EC 1.1.99.16 malate dehydrogenase (acceptor | EC 1.2.1.18 malonate-semialdehyde dehydrogenase |
| EC 1 1.3.37 D-arabinono- 1,4-lactone oxidase | EC 1 1.99.17 glucose dehydrogenase | (acetylating) |
| EC 1.1.3.38 vanillyl-alcohol oxidase | (pyrroloquinoline-quinone) | EC 1.2.1.19 aminobutyraldehyde dehydrogenase |
| EC 1.1.3.39 nucleoside oxidase (H₂O₂-forming) | EC 1.1.99.18 cellobiose dehydrogenase (acceptor) | EC 1.2.1.20 glutarate-semialdehyde dehydrogenase |
| | EC 1.1.99.19 uracil dehydrogenase | EC 1.2.1.21 glycolaldehyde dehydrogenase |
| | EC 1.1.99.20 alkan-1-ol dehydrogenase (acceptor) | EC 1.2.1.22 lactaldehyde dehydrogenase |
| EC 1.1.4 | EC 1.1.99.21 D-sorbitol dehydrogenase (acceptor) | EC 1.2.1.23 2-oxoaldehyde dehydrogenase (AND) |
| | EC 1.1.99.22 glycerol dehydrogenase (acceptor) | EC 1.2.1.24 succinate-semialdehyde dehydrogenase |
| WITH A DISULFIDE AS ACCEPTOR | EC 1.1.99.23 polyvinyl-alcohol dehydrogenase | EC 1.2.1.25 2-oxoisovalerate dehydrogenase |
| | (acceptor) | (acylating) |
| EC 1.1.4.1 vitamin-K-epoxide reductase (warfarin- | EC 1.1.99.24 hydroxyacid-oxoacid transhydrogenase | EC 1.2.1.26 2,5-dioxovalerate dehydrogenase |
| sensitive) | EC 1.1.99.25 quinate dehydrogenase | EC 1.2.1.27 methylmalonate-semialdehyde |
| EC 1.1.4.2 vitamin-K-epoxide reductase (warfarin- | (pyrroloquinoline-quinone) | dehydrogenase (acylating) |
| insensitive) | EC 1 1.99.26 3-hydroxycyclohexanone dehydrogenase | EC 1.2.1.28 benzaldehyde dehydrogenase (AND) |
| | EC 1.1.99.27 (R)-pantolactone dehydrogenase (flavin) | EC 1.2.1.29 aryl-aldehyde dehydrogenase |
| | EC 1.1.99.28 glucose-fructose oxidoreductase | EC 1.2.1.30 aryl-aldehyde dehydrogenase (NADP) |
| EC 1.1.5 | | EC 1.2.1.31 L-aminoadipate-semialdehyde |
| WITH A QUINONE OR SIMILAR COMPOUND AS | EC 1.2.1 | dehydrogenase |
| ACCEPTOR | | EC 1.2.1.32 aminomuconate-semialdehyde |
| EC 1.1.5.1 cellobiose dehydrogenase (quinone) | dehydrogenase | EC 1.2.1.33 (R)-dehydropantoate dehydrogenase |
| | WITH AND OR NADP AS ACCEPTOR | EC 1.2.1.34 deleted, included in EC 1.1.1.131 |
| | EC 1.2.1.1 formaldehyde dehydrogenase (glutathione) | EC 1.2.1.35 now EC 1.1.1.203 |
| EC 1.1.99 | EC 1.2.1.2 formate dehydrogenase | EC 1.2.1.36 retinal dehydrogenase |
| | EC 1.2.1.3 aldehyde dehydrogenase (AND) | EC 1.2.1.37 now EC 1.1.1.204 |
| | EC 1.2.1.4 aldehyde dehydrogenase (NADP) | EC 1.2.1.38 N-acetyl-γ-glutamyl-phosphate reductase |
| WITH OTHER ACCEPTORS | EC 1.2.1.5 aldehyde dehydrogenase [AND(P)] | EC 1.2.1.39 phenylacetaldehyde dehydrogenase |
| | EC 1.2.1.6 deleted | EC 1.2.1.40 3α, 7α, 12α-trihydroxycholestan-26-al 26- |
| EC 1.1.99.1 choline dehydrogenase | EC 1.2.1.7 benzaldehyde dehydrogenase (NADP) | oxidoreductase |
| EC 1.1.99.2 2-hydroxyglutarate dehydrogenase | EC 1.2.1.8 betaine.aldehyde dehydrogenase | EC 1.2.1.41 glutamate-5-semialdehyde dehydrogenase |
| EC 1.1.99.3 gluconate 2-dehydrogenase | EC 1.2.1.9 glyceraldehyde-3-phosphate | EC 1.2.1.42 hexadecanal dehydrogenase (acylating) |
| EC 1.1.99.4 dehydrogluconate dehydrogenase | dehydrogenase (NADP) | EC 1.2.1.43 formate dehydrogenase (NADP) |
| EC 1.1.99.5 glycerol-3-phosphate dehydrogenase | EC 1.2.1.10 acetaldehyde dehydrogenase (acetylating) | EC 1.2.1.44 cinnamoyl-CoA reductase |
| EC 1.1.99.6 D-2-hydroxy-acid dehydrogenase | EC 1.2.1.11 aspartate-semialdehyde dehydrogenase | EC 1.2.1.45 40carboxy-2-hydroxymuconate-6- |
| EC 1.1.99.7 lactate-malate transhydrogenase | EC 1.2.1.12 glyceraldehyde-3-phosphate | semialdehyde dehydrogenase |
| EC 1.1.99.8 alcohol dehydrogenase (acceptor) | dehydrogenase (phosphorylating) | EC 1.2.1.46 formaldehyde dehydrogenase |
| EC 1.1.99.9 pyridoxine 5-dehydrogenase | EC 1.2.1.13 glyceraldehyde-3-phosphate | EC 1.2.1.47 4-trimethylammoniobutyraldehyde |
| EC 1.1.99.10 glucose dehydrogenase (acceptor) | dehydrogenase (NADP) (phosphorylating) | dehydrogenase |
| EC 1.1.99.11 fructose 5-dehydrogenase | EC 1.2.1.14 now EC 1.1.1.205 | EC 1.2.1.48 long-chain-aldehyde dehydrogenase |
| EC 1.1.99.12 sorbose dehydrogenase | EC 1.2.1.15 malonate-semialdehyde dehydrogenase | EC 1.2.1.49 2-oxoaldehyde dehydrogenase (NADP) |
| EC 1.1.99.13 glucoside 3-dehydrogenase | EC 1.2.1.16 succinate-semialdehyde dehydrogenase | EC 1.2.1.50 long-chain-fatty-acyl-CoA reductase |
| EC 1.1.99.14 glycolate dehydrogenase | [AND(P)] | EC 1.2.1.51 pyruvate dehydrogenase (NADP) |
| EC 1.1.99.15 now EC 1.7.99.5 | EC 1.2.1.17 glyoxylate dehydrogenase (acylating) | EC 1.2.1.52 oxoglutarate dehydrogenase (NADP) |
| EC 1.2.1.53 4-hydroxyphenylacetaldehyde | EC 1.2.3.4 oxalate oxidase | |
| dehydrogenase | EC 1.2.3.5 glyoxylate oxidase | |
| EC 1.2.1.54 γ-guanidinobutyraldehyde dehydrogenase | EC 1.2.3.6 pyruvate oxidase (CoA-acetylating) | EC 1.3.1 |
| EC 1.2.1.55 (R)-3-hydroxyacid ester dehydrogenase | EC 1.2.3.7 indole-3-acetaldehyde oxidase | |
| EC 1.2.1.56 (S)-3-hydroxyacid ester dehydrogenase | EC 1.2.3.8 pyridoxal oxidase | WITH AND OR NADP AS ACCEPTOR |
| EC 1.2.1 57 butanal dehydrogenase | EC 1.2.3.9 aryl-aldehyde oxidase | |
| EC 1.2.1.58 phenylglyoxylate dehydrogenase | EC 1.2 3.10 carbon-monoxide oxidase | EC 1.3.1 |
| (acylating) | EC 1.2.3.11 retinal oxidase | EC 1.3.1.1 dihydrouracil dehydrogenase (AND) |
| EC 1.2.1.59 glyceraldehyde-3-phosphate | EC 1.2.3.12 vanillate demethylase | EC 1.3.1.2 dihydropyrimidine dehydrogenase (NADP) |
| dehydrogenase (AND(P)) (phosphorylating) | EC 1.2.3.13 4-hydroxyphenylpyruvate oxidase | EC 1.3.1.3 cortisone β-reductase |
| EC 1.2.1 60 5-carboxymethyl-2-hydroxymuconic- | | EC 1.3.1.4 cortisone α-reductase |
| semialdehyde dehydrogenase | | EC 1.3.1.5 cucurbitacin Δ²³-reductase |
| EC 1.2.1.61 4-hydroxymuconic semialdehyde | EC 1.2.4 | EC 1.3.1.6 fumarate reductase (NADH₂) |
| dehydrogenase | | EC 1.3.1.7 meso-tartrate dehydrogenase |
| EC 1 2.1.62 4-formylbenzenesulfonate dehydrogenase | WITH A DISULFIDE AS ACCEPTOR | EC 1.3.1.8 acyl-CoA dehydrogenase (NADP) |
| EC 1.2.1.63 6-oxohexanoate dehydrogenase | | EC 1.3.1.9 enoyl-[acyl-carrier-protein] reductase |
| EC 1.2.1.64 4-hydroxybenzaldehyde dehydrogenase | EC 1.2.4.1 pyruvate dehydrogenase (lipoamide) | (NADH₂) |
| EC 1.2.1.65 salicylaldehyde dehydrogenase | EC 1.2.4.2 oxoglutarate dehydrogenase (lipoamide | EC 1.3.1.10 enoyl-[acyl-carrier-protein] reductase |
| EC 1.2.1.66 mycothiol-dependent formaldehyde | EC 1.2.4.3 deleted, included in EC 1.2.4.4 | (NADPH₂, B-specific) |
| dehydrogenase | EC 1.2.4.4 3-methyl-2-oxobutanoate dehydrogenase | EC 1.3.1.11 coumarate reductase |
| EC 1.2.1.67 vanillin dehydrogenase | (lipoamide) | EC 1.3.1.12 prephenate dehydrogenase |
| EC 1.2.1.68 coniferyl-aldehyde dehydrogenase | | EC 1.3.1.13 prephenate dehydrogenase (NADP) |
| | | EC 1.3.1.14 orotate reductase (NADH₂) |
| | EC 1.2.7 | EC 1.3.1.15 orotate reductase (NADPH₂) |
| EC 1.2.2 | EC 1.3.1.16 β-nitroacrylate reductase | |
| | WITH AN IRON-SULFUR PROTEIN AS ACCEPTOR | EC 1.3.1.17 3-methyleneoxindole reductase |
| WITH A CYTOCHROME AS ACCEPTOR | EC 1.2.7.1 pyruvate synthase | EC 1.3.1.18 kynurenate-7,8-dihydrodiol |
| EC 1.2.2.1 formate dehydrogenase (cytochrome) | EC 1.2.7.2 2-oxobutyrate synthase | dehydrogenase |
| EC 1.2.2.2 pyruvate dehydrogenase (cytochrome) | EC 1.2.7.3 2-oxoglutarate synthase | EC 1.3.1.19 cis-1,2-dihydrobenzene-1,2-diol |
| EC 1.2.2.3 formate dehydrogenase (cytochrome-c- | | dehydrogenase |
| 553) | | EC 1.3.1.20 trans-1,2-dihydrobenzene-1,2-diol |
| EC 1.2.2.4 carbon-monoxide oxygenase (cytochrome | EC 1.2.99 | dehydrogenase |
| b-561) | | EC 1.3.1.21 7-dehydrocholesterol reductase |
| | WITH OTHER ACCEPTORS | EC 1.3.1.22 cholestenone 5α-reductase |
| EC 1.2.3 | EC 1.2.99.1 now EC 1.1.99.19 | EC 1.3.1.23 cholestenone 5β-reductase |
| | EC 1.2.99.2 carbon-monoxide dehydrogenase | EC 1.3.1.24 biliverdin reductase |
| WITH OXYGEN AS ACCEPTOR | EC 1.2.99.3 aldehyde dehydrogenase | EC 1.3.1.25 1,6-dihydroxycyclohexa-2,4-diene-1- |
| | (pyrroloquinoline-quinone) | carboxylate dehydrogenase |
| EC 1.2.3.1 aldehyde oxidase | EC 1.2.99.4 formaldehyde dismutase | EC 1.3.1.26 dihydrodipicolinate reductase |
| EC 1.2.3.2 now EC 1.1.3.22 | EC 1.2.99.5 formylmethanofuran dehydrogenase | EC 1.3.1.27 2-hexadecenal reductase |
| EC 1.2.3.3 pyruvate oxidase | EC 1.2.99.6 carboxylate reductase | EC 1.3.1.28 2,3-dihydro-2,3-dihydroxybenzoate |
| | | dehydrogenase |
| | | EC 1.3.1.29 cis-1,2-dihydro-1,2-dihydroxynaphthalene |
| dehydrogenase | dehydrogenase | EC 1.3.5 |
| EC 1.3.1.30 progesterone 5α-reductase | EC 1 3.1.62 pimeloyl-CoA dehydrogenase | |
| EC 1.3.1.31 2-enoate reductase | EC 1.3.1.63 2,4-dichlorobenzoyl-CoA reductase | WITH A QUINONE OR RELATED COMPOUND AS |
| EC 1.3.1.32 maleylacetate reductase | EC 1.3.1.64 phthalate 4,5-cis-dihydrodiol | ACCEPTOR |
| EC 1.3.1.33 protochlorophyllide reductase | dehydrogenase | |
| EC 1.3.1.34 2,4-dienoyl-CoA reductase (NADPH₂) | EC 1.3.1.65 5,6-dihydroxy-3-methyl-2-oxo-1,2,5,6- | EC 1.3.5.1 succinate dehydrogenase (ubiquinone) |
| EC 1.3.1.35 phosphatidylcholine desaturase | tetrahydroquinoline dehydrogenase | |
| EC 1.3.1.36 geissoschizine dehydrogenase | EC 1.3.1.66 cis-dihydroethyicatechol dehydrogenase | |
| EC 1.3 1.37 cis-2-enoyl-CoA reductase (NADPH₂) | EC 1.3.1.67 cis-1,2-dihydroxy-4-methylcyclohexa-3,5- | EC 1.3.7 |
| EC 1.3.1.38 trans-2-enoyl-CoA reductase (NADPH₂) | diene-1-carboxylate dehydrogenase | |
| EC 1.3.1.39 enoyl-[acyl-carrier-protein] reductase | EC 1.3.1.68 1,2-dihydroxy-6-methylcyclohexa-3,5- | WITH AN IRON-SULFUR PROTEIN AS ACCEPTOR |
| (NADPH₂, A-specific) | dienecarboxylate dehydrogenase | |
| EC 1.3.1.40 2-hydroxy-6-oxo-6-phenylhexa-2,4- | EC 1.3.1.69 zeatin reductase | EC 1.3.7.1 6-hydroxynicotinate reductase |
| dienoate reductase | | |
| EC 1.3.1.41 xanthommatin reductase | | |
| EC 1.3.1.42 12-oxophytodienoate reductase | EC 1.3.2 | EC 1.3.99 |
| EC 1.3.1.43 cyclohexadienyl dehydrogenase | | |
| EC 1.3 1.44 trans-2-enoyl-CoA reductase(AND) | WITH A CYTOCHROME AS ACCEPTOR | WITH OTHER ACCEPTORS |
| EC 1.3.1.45 2-hydroxyisoflavone reductase | | |
| EC 1.3 1.46 biochanin-A reductase | EC 1.3.2.1 now EC 1.3.99.2 | EC 1.3.99.1 succinate dehydrogenase |
| EC 1.3.1.47 α-santonin 1,2-reductase | EC 1.3.2.2 now EC 1 3.99.3 | EC 1.3.99.2 butyryl-CoA dehydrogenase |
| EC 1.3 1.48 15-oxoprostaglandin | EC 1.3.2.3 galactonolactone dehydrogenase | EC 1.3 99.3 acyl-CoA dehydrogenase |
| EC 1.3 1 49 cis-3,4-dihydrophenanthrene-3,4-diol | | EC 1.3.99.4 3-oxosteroid 1-dehydrogenase |
| dehydrogenase | | EC 1.3.99.5 3-oxo-5α-steroid 4-dehydrogenase |
| EC 1.3.1.50 now EC 1.1.1.252 | EC 1.3.3 | EC 1.3.99.6 3-oxo-5β-steroid 4-dehydrogenase |
| EC 1.3.1.51 2-hydroxydaidzein reductase | | EC 1.3.99.7 glutaryl-CoA dehydrogenase |
| EC 1.3.1.52 2-methyl-branched-chain-enoyl-CoA | WITH OXYGEN AS ACCEPTOR | EC 1.3.99.8 2-furoyl-CoA dehydrogenase |
| reductase | | EC 1.3.99.9 β-cyclopiazonate dehydrogenase |
| EC 1.3.1.53 (3S,4R)-3,4-dihydroxycyclohexa-1,5-diene- | EC 1.3.3.1 dihydroorotate oxidase | EC 1.3.99.10 isovaleryl-CoA dehydrogenase |
| 1,4-dicarboxylate dehydrogenase | EC 1.3.3.2 lathosterol oxidase | EC 1.3.99.11 dihydroorotate dehydrogenase |
| EC 1.3.1.54 precorrin-6X reductase | EC 1.3.3.3 coproporphyrinogen oxidase | EC 1.3.99.12 2-methylacyl-CoA dehydrogenase |
| EC 1.3.1.55 cis-1,2-dihydroxycyclohexa-3,5-diene-1- | EC 1.3.3.4 protoporphyrinogen oxidase | EC 1.3.99.13 long-chain-acyl-CoA dehydrogenase |
| carboxylate dehydrogenase | EC 1.3.3.5 bilirubin oxidase | EC 1.3.99.14 cylohexanone dehydrogenase |
| EC 1.3.1.56 cis-2,3-dihydrobiphenyl-2,3-diol | EC 1.3.3.6 acyl-CoA oxidase | EC 1.3.99.15 benzoyl-CoA reductase |
| dehydrogenase | EC 1.3.3.7 dihydrouracil oxidase | EC 1.3.99.16 isoquinoline 1-oxidoreductase |
| EC 1.3.1 57 phioroglucinol reductase | EC 1.3.3.8 tetrahydroberberine oxidase | EC 1.3.99.17 quinoline 2-oxidoreductase |
| EC 1.3.1.58 2,3-dihydroxy-2,3-dihydro-p-cumate | | EC 1.3.99.18 quinaldate 4-oxidoreductase |
| dehydrogenase | | EC 1.3.99.19 quinoline-4-carboxylate 2- |
| EC 1.3.1.59 1,6-dihydroxy-5-methylcyclohexa-2,4- | | oxidoreductase |
| dienecarboxylate dehydrogenase | | EC 1.3.99.20 4-hydroxybenzoyl-CoA reductase |
| EC 1.3.1.60 dibenzothiophene dihydrodiol | | |
| dehydrogenase | | |
| EC 1.3.1.61 terephthalate 1,2-cis-dihydrodiol | | |
| EC 1.4.1 | EC 1.4.3.6 amine oxidase (copper-containing) | EC 1.5.1 |
| | EC 1.4.3 7 D-glutamate oxidase | |
| WITH AND OR NADP AS ACCEPTOR | EC 1.4.3.8 ethanolamine oxidase | WITH AND OR NADP AS ACCEPTOR |
| | EC 1.4.3.9 deleted, included in EC 1.4.3.4 | |
| EC 1.4.1.1 alanine dehydrogenase | EC 1.4.3.10 putrescine oxidase | EC 1.5.1.1 pyrroline-2-carboxylate reductase |
| EC 1.4.1.2 glutamate dehydrogenase | EC 1.4.3.11 L-glutamate oxidase | EC 1.5.1.2 pyrroline-5-carboxylate reductase |
| EC 1.4.1.3 glutamate dehydrogenase [AND(P)] | EC 1.4.3.12 cyclohexylamine oxidase | EC 1.5.1.3 dihydrofolate reductase |
| EC 1.4.1.4 glutamate dehydrogenase (NADP) | EC 1.4.3.13 protein-lysine 6-oxidase | EC 1.5.1.4 deleted, included in EC 1.5.1.3 |
| EC 1.4.1.5 L-amino-acid dehydrogenase | EC 1.4.3.14 L-lysine oxidase | EC 1.5.1.5 methylenetetrahydrofolate dehydrogenase |
| EC 1.4.1.6 deleted, included in EC 1.4.4.1 | EC 1.4.3.15 D-glutamate(D-aspartate) oxidase | (NADP) |
| EC 1.4.1.7 serine dehydrogenase | EC 1.4.3.16 L-aspartate oxidase | EC 1.5.1.6 formyltetrahydrofolate dehydrogenase |
| EC 1.4.1.8 valine dehydrogenase (NADP) | EC 1.4.3.17 tryptophan α,β-oxidase | EC 1.5.1.7 saccharopine dehydrogenase (AND,L- |
| EC 1.4.1.9 leucine dehydrogenase | EC 1.4.3.18 cytokinin oxidase proposed addition | lysine-forming) |
| EC 1.4.1.10 glycine dehydrogenase | | EC 1.5.1.8 saccharopine dehydrogenase (NADP,L- |
| EC 1.4.1.11 L-erythro-3,5-diaminohexanoate | | lysine-forming) |
| EC 1.4.1.12 2,4-diaminopentanoate dehydrogenase | EC 1.4.4 | EC 1.5.1.9 saccharopine dehydrogenase (AND,L- |
| EC 1.4.1.13 glutamate synthase (NADPH₂) | | glutamate-forming) |
| EC 1.4.1.14 glutamate synthase (NADH₂) | WITH A DISULFIDE AS ACCEPTOR | EC 1.5.1.10 saccharopine dehydrogenase (NADP,L- |
| EC 1.4.1.15 lysine dehydrogenase | EC 1.4.1.18 lysine 6-dehydrogenase | glutamate-forming) |
| EC 1.4.1.19 tryptophan dehydrogenase | EC 1.4.4.1 D-proline reductase (dithiol) | EC 1.5.1.11 D-octopine dehydrogenase |
| EC 1.4.1.20 phenylalanine dehydrogenase | EC 1.4.4.2 glycine dehydrogenase (decarboxylating) | EC 1.5.1.12 1-pyrroline-5-carboxylate dehydrogenase |
| | | EC 1.5.1.13 nicotinate dehydrogenase |
| | | EC 1.5.1.14 deleted, included in EC 1 5.1 21 |
| | EC 1.4.7 | EC 1.5.1.15 methylenetetrahydrofolate dehydrogenase |
| EC 1.4.2 | | (AND) |
| | WITH AN IRON-SULFUR PROTEIN AS ACCEPTOR | EC 1.5.1.16 D-lysopine dehydrogenase |
| | | EC 1.5.1.17 alanopine dehydrogenase |
| WITH A CYTOCHROME AS ACCEPTOR | EC 1.4.7.1 glutamate synthase (ferredoxin | EC 1.5.1.18 ephedrine dehydrogenase |
| | | EC 1.5.1.19 D-nopaline dehydrogenase |
| EC 1.4.2.1 glycine dehydrogenase (cytochrome) | EC 1.5.1.20 methylenetetrahydrofolate reductase | EC 1.4.99.2 taurine dehydrogenase |
| | EC 1.4.99 | (NADPH₂) |
| | | EC 1.5.1.21 Δ¹-piperideine-2-carboxylate reductase |
| EC 1.4.3 | | EC 1.5.1.22 strombine dehydrogenase |
| | WITH OTHER ACCEPTORS | EC 1.5.1.23 tauropine dehydrogenase |
| WITH OXYGEN AS ACCEPTOR | | EC 1.5.1.24 N⁵-(carboxyethyl)ornithine synthase |
| | EC 1.4.99.1 D-amino-acid dehydrogenase | EC 1.5.1.25 thiomorpholine-carboxylate |
| EC 1.4.3.1 D-aspartate oxidase | EC 1.4.99.3 amine dehydrogenase | dehydrogenase |
| EC 1.4.3.2 L-amino-acid oxidase | EC 1.4.99.4 aralkylamine dehydrogenase | EC 1.5.1.26 β-alanopine dehydrogenase |
| EC 1.4.3.3 D-amino-acid oxidase | | EC 1.5.1.27 1,2-dehydroreticulinium reductase |
| EC 1.4.3.4 amine oxidase (flavin-containing) | | (NADPH₂) |
| EC 1.4.3.5 pyridoxamine-phosphate oxidase | | EC 1.5 1.28 opine dehydrogenase |
| EC 1.5.3 | EC 1.5.99.6 spermidine dehydrogenase | EC 1.6.4.9 Bis-γ-glutamylcystine reductase (NADPH) |
| | EC 1.5.99.7 trimethylamine dehydrogenase | EC 1.6.4.10 CoA-disulfide reductase (NADH₂) |
| WITH OXYGEN AS ACCEPTOR | EC 1.5.99.8 proline dehydrogenase | |
| | EC 1.5.99.9 methylenetetrahydromethanopterin | |
| EC 1.5.3.1 sarcosine oxidase | dehydrogenase | EC 1.6.5 |
| EC 1.5.3.2 N-methyl-L-amino-acid oxidase | EC 1.5.99.10 dimethylamine dehydrogenase | |
| EC 1.5.3.3 deleted | EC 1.5.99.11 coenzyme F₄₂₀-dependent N⁵,N¹⁰- | WITH A QUINONE OR SIMILAR COMPOUND AS |
| EC 1.5.3.4 N⁶-methyl-lysine oxidase | methenyltetrahydromethanopterin reductase | ACCEPTOR |
| EC 1.5.3.5 (S)-6-hydroxynicotine oxidase | | |
| EC 1.5.3.6 (R)-6-hydroxynicotine oxidase | | EC 1.6.5.1 deleted |
| EC 1.5.3.7 L-pipecolate oxidase | EC 1.6.1 | EC 1.6.5.2 now EC 1.6.99.2 |
| EC 1.5.3.8 deleted, included in EC 1.3.3.8 | | EC 1.6.5.3 NADH₂ dehydrogenase (ubiquinone) |
| EC 1.5 3.9 reticuline oxidase | WITH AND OR NADP AS ACCEPTOR | EC 1.6.5.4 monodehydroascorbate reductase (NADH₂) |
| EC 1.5.3.10 dimethylglycine oxidase | | EC 1.6.5.5 NADPH:quinone reductase |
| EC 1.5.3.11 polyamine oxidase | EC 1.6.1.1 AND(P) transhydrogenase (B-specific) | EC 1.6.5.6 p-benzoquinone reductase (NADPH) |
| EC 1.5.3.12 dihydrobenzophenanthridine oxidase | EC 1.6.1.2 AND(P) transhydrogenase (AB-specific) | EC 1.6.5.7 2-hydroxy-1,4-benzoquinone reductase |
| EC 1.5.4 | EC 1.6.2 | EC 1.6.6 |
| WITH A DISULFIDE AS ACCEPTOR | WITH A HEME PROTEIN AS ACCEPTOR | WITH A NITROGENOUS GROUP AS ACCEPTOR |
| EC 1.5.4.1 pyrimidodiazepine synthase | EC 1.6.2.1 now EC 1.6.99.3 | EC 1.6.6.1 nitrate reductase (NADH₂) |
| | EC 1 6.2.2 cytochrome-b₅ reductase | EC 1.6.6.2 nitrate reductase [AND(P)H₂] |
| | EC 1.6.2.3 deleted | EC 1.6.6.3 nitrate reductase (NADPH₂) |
| EC 1.5.5 | EC 1.6.2 4 NADPH-ferrihemoprotein reductase | EC 1.6.6.4 nitrate reductase [AND(P)H₂] |
| | EC 1.6.2.5 NADPH-cytochrome-c₂ reductase | EC 1.6.6.5 now EC 1.7.99.3 |
| WITH A QUINONE OR SIMILAR COMPOUND AS | EC 1.6.2.6 leghemoglobin reductase | EC 1.6.6.6 hyponitrite reductase |
| ACCEPTOR | | EC 1.6.6.7 azobenzene reductase |
| | | EC 1.6.6.8 GMP reductase |
| EC 1.5.5.1 electron-transferring-flavoprotein | EC 1.6.4 | EC 1.6.6.9 trimethylamine-N-oxide reductase |
| dehydrogenase | | EC 1.6.6.10 nitroquinoline-N-oxide reductase |
| | WITH A DISULFIDE AS ACCEPTOR | EC 1.6.6.11 hydroxylamine reductase (NADH₂) |
| | | EC 1 6.6.12 4-(dimethylamino)phenylazoxybenzene |
| EC 1.5.99 | EC 1.6.4 1 cystine reductase (NADH₂) | reductase |
| | EC 1.6.4.2 glutathione reductase (NADPH) | EC 1 6.6.13 N-hydroxy-2-acetamidofluorene reductase |
| WITH OTHER ACCEPTORS | EC 1.6.4.3 now EC 1.8 1.4 | |
| | EC 1.6.4.4 protein-disulfide reductase [AND(P)H] | |
| EC 1.5.99.1 sarcosine dehydrogenase | EC 1 6.4.5 thioredoxin reductase (NADPH) | |
| EC 1.5.99.2 dimethylglycine dehydrogenase | EC 1.6.4.6 CoA-glutathione reductase (NADPH) | |
| EC 1.5.99 3 L-pipecolate dehydrogenase | EC 1.6.4.7 asparagusate reductase (NADH₂) | |
| EC 1.5.99.4 nicotine dehydrogenase | EC 1.6.4.8 trypanothione reductase | |
| EC 1.5.99.5 methylglutamate dehydrogenase | | |
| EC 1.6.8 | EC 1.7.3.4 hydroxylamine oxidase | EC 1.8.3 |
| | EC 1.7.3 5 3-aci-nitropropanoate oxidase | |
| WITH A FLAVIN AS ACCEPTOR | | |
| EC 1.6.8.1 AND(P)H₂ dehydrogenase (FMN) | | WITH OXYGEN AS ACCEPTOR |
| EC 1.6.8.2 NADPH₂ dehydrogenase (flavin) | EC 1.7.7 | EC 1.8.3.1 sulfite oxidase |
| | | EC 1.8.3.2 thiol oxidase |
| | | EC 1.8.3.3 glutathione oxidase |
| EC 1.7.99 | WITH AN IRON-SULFUR PROTEIN AS ACCEPTOR | EC 1.8.3.4 methanethiol oxidase |
| | EC 1.7.7.1 ferredoxin-nitrate reductase | |
| WITH OTHER ACCEPTORS | EC 1.7.7.2 ferredoxin-nitrate reductase | EC 1.8.4 |
| EC 1.6.99 1 NADPH₂ dehydrogenase | | |
| EC 1.6.99.2 AND(P)H₂ dehydrogenase (quinone) | EC 1.7.99 | WITH A DISULFIDE AS ACCEPTOR |
| EC 1.6.99.3 NADH₂ dehydrogenase | | |
| EC 1.6.99.4 now EC 1.18.1.2 | | EC 1.8.4.1 glutathione-homocystine transhydrogenase |
| EC 1 6.99.5 NADH₂ dehydrogenase (quinone) | WITH OTHER ACCEPTORS | EC 1.8.4.2 protein-disulfide reductase (glutathione) |
| EC 1.6.99.6 NADPH₂ dehydrogenase (quinone) | | EC 1.8.4.3 glutathione-CoA-glutathione |
| EC 1.6.99.7 dihydropteridine reductase | EC 1.7 99.1 hydroxylamine reductase | transhydrogenase |
| EC 1.6.99.8 aquacobalamin reductase | EC 1.7 99.2 deleted | EC 1.8.4.4 glutathione-cystine transhydrogenase |
| EC 1.6.99.9 cob(ll)alamin reductase | EC 1.7 99.3 nitrite reductase | EC 1.8.4.5 methionine-S-oxide reductase |
| EC 1.6.99.10 deleted, included in EC 1.6.99.7 | EC 1.7 99.4 nitrate reductase | EC 1.8.4.6 protein-methionine-S-oxide reductase |
| EC 1.6.99.11 aquacobalamin reductase (NADPH) | EC 1.7.99.5 5,10-methylenetetrahydrofolate reductase | EC 1.8.4.7 enzyme-thiol transhydrogenase (oxidized- |
| EC 1.6.99.12 cyanocobalamin reductase(NADPH, | (FADH₂) | (glutathione) |
| cyanide-eliminating) | EC 1.8.1 | EC 1.8.4.8 phosphoadenylyl-sulfate reductase |
| EC 1.6.99.13 ferric-chelate reductase | | (thioredoxin) |
| | EC 1.8.1 | EC 1.8.4.9 adenylyl-sulfate reductase (glutathione) |
| | EC 1.8.1.3 hypotaurine dehydrogenase | |
| EC 1.7.2 | | |
| WITH A CYTOCHROME AS ACCEPTOR | WITH AND OR NADP AS ACCEPTOR | EC 1.8.5 |
| EC 1.7.2.1 nitrite reductase (cytochrome) | EC 1.8.1.1 deleted | |
| | EC 1.8.1.2 dulfite reductase (NADPH₂) | WITH A QUINONE OR SIMILAR COMPOUND AS |
| | EC 1.8.1.3 hypotaurine dehydrogenase | ACCEPTOR |
| EC 1.7.3 | EC 1.8.1.4 dihydrolipoamide dehydrogenase | |
| | | EC 1.8.5.1 glutathione dehydrogenase (ascorbate) |
| WITH OXYGEN AS ACCEPTOR | EC 1.8.2 | |
| | | EC 1.8.7 |
| EC 1.7.3.1 nitroethane oxidase | | |
| EC 1.7.3.2 acetylindoxyl oxidase | WITH A CYTOCHROME AS ACCEPTOR | |
| EC 1 7.3.3 urate oxidase | | WITH AN IRON-SULFUR PROTEIN AS ACCEPTOR |
| | EC 1.8.2.1 sulfite dehydrogenase | |
| | EC 1.8.2 2 thiosulfate dehydrogenase | EC 1.8.7.1 sulfite reductase (ferredoxin) |
| EC 1.8.99 | EC 1.10.2 | EC 1.11.9 glutathioe peroxidase |
| | | EC 1.11.10 chloride peroxidase |
| | | EC 1.11.11 L-ascorbate peroxidase |
| WITH OTHER ACCEPTORS | WITH A CYTOCHROME AS ACCEPTOR | EC 1.11.12 phopholipid-hydroperoxide glutathione |
| | | peroxidase |
| EC 1.8.99.1 sulfite reductase | EC 1.10.2.1 L-ascorbate-cytochrome-b₅ reductase | EC 1.11.13 manganese peroxidase |
| EC 1.8.99.2 adenylyl-sulfate reductase | EC 1.10.2 2 ubiquinol-cytochrome-c reductase | EC 1.11.14 diarylpropane peroxidase |
| EC 1.8.99.3 hydrogensulfite reductase | | |
| EC 1.8.99.4 now EC 1.8.4.8 | | |
| | EC 1.10.3 | EC 1.12.1 |
| EC 1.9.3 | | |
| EC 1.10.3.3 L-ascorbate oxidase | WITH OXYGEN AS ACCEPTOR | WITH AND OR NADP AS ACCEPTOR |
| EC 1.10.3.4 o-aminophenol oxidase | | |
| WITH OXYGEN AS ACCEPTOR | EC 1.10.3.1 catechol oxidase | EC 1.12.1 1 now EC 1.18 99.1 |
| | EC 1.10.3.2 laccase | EC 1.12.1.2 hydrogen dehydrogenase |
| EC 1.9.3.1 cytochrome-c oxidase | EC 1.10.3.3 L-ascorbate oxidase | |
| EC 1.9.3.2 Pseudomonas cytochrome oxidase | EC 1.10.3.4 o-aminophenol oxidase | |
| | EC 1.10.3.5 3-hydroxyanthranilate oxidase | EC 1.12.2 |
| | EC 1.10.3.6 rifamycin-B oxidase | |
| EC 1.9.6 | EC 1.10.3.7 sulochrin oxidase [(+)-bisdechlorogeodin- | |
| | forming] | WITH A CYTOCHROME AS ACCEPTOR |
| | EC 1.10.3.8 sulochrin oxidase [(·)-bisdechlorogeodin- | |
| WITH A NITROGENOUS GROUP AS ACCEPTOR | forming] | EC 1.12.2.1 cytochrome-c₃ hydrogenase |
| EC 1.9.6.1 nitrate reductase (cytochrome) | | |
| | EC 1.10.99 | 1.12.99 |
| EC 1.9.99 | | |
| | WITH OTHER ACCEPTORS | WITH OTHER ACCEPTORS |
| WITH OTHER ACCEPTORS | EC 1.10.99.1 plastoquinol-plastocyanin reductase | EC 1.12.99.1 coenzyme F420 hydrogenase |
| | | EC 1.12.99.2 coenzyme-M-7- |
| EC 1 9.99 1 iron-cytochrome-c reductase | | mercaptoheptanoylthreonine-phosphate- |
| | EC 1.11.1 | heterodisulfide hydrogenase |
| | | EC 1.12.99.3 hydrogen:quinone oxidoreducatse |
| EC 1.10.1 | | EC 1.12.99.4 N⁵,N¹⁰- |
| | PEROXIDASES | methenyltetrahydromethanopterin hydrogenase |
| | | EC 1.12.99.5 3,4-dihydroxyquinoline 2,4-dioxygenase |
| WITH AND OR NADP AS ACCEPTOR | EC 1.11.1.1 NADH₂ peroxidase | |
| | EC 1.11.1.2 NADPH₂ peroxidase | |
| EC 1 10.1.1 trans-acenaphthene-1,2-diol | EC 1.11.1.3 fatty-acid peroxidase | |
| dehydrogenase | EC 1.11.1.4 now EC 1.13.11.11 | |
| EC 1.11.1.5 cytochrome-c peroxidase | | |
| | EC 1.11.1.6 catalase | |
| | EC 1.11.1.7 peroxidase | |
| | EC 1.11.1.8 iodide peroxidase | |
| EC 1.13.11 | EC 1.13.11.36 chloridazon-catechol dioxygenase | EC 1.13.99.4 now EC 1.14.12.9 |
| | EC 1.13.11.37 hydroxyquinol 1,2-dioxygenase | EC 1.13.99.5 3,4-dihydroxyquinoline 2,4-dioxygenase |
| | EC 1.13.11.38 1-hydroxy-2-naphthoate 1,2- | |
| \WITH INCORPORATION OF TWO ATOMS OF OXYGEN | dioxygenase | |
| | EC 1.13.11.39 biphenyl-2,3-diol 1,2-dioxygenase | EC 1.14.11 |
| EC 1.13.11.1 catechol 1,2-dioxygenase | EC 1.13.11.40 arachidonate 8-lipoxygenase | |
| EC 1.13.11.2 catechol 2,3-dioxygenase | EC 1.13.11.41 2,4′-dihydroxyacetophenone | |
| EC 1.13.11,3 protocatechuate 3,4-dioxygenase | dioxygenase | WITH 2-OXOGLUTARATE AS ONE DONOR, AND |
| EC 1.13.11.4 gentisate 1,2-dioxygenase | EC 1.13.11.42 indoleamine-pyrrole 2,3-dioxygenase | INCORPORATION OF ONE ATOM EACH OF OXYGEN |
| EC 1.13.11.5 homogentisate 1,2-dioxygenase | EC 1.13.11.43 lignostilbene αβ-dioxygenase | INTO BOTH DONORS |
| EC 1.13.11.6 3-hydroxyanthranilate 3,4-dioxygenase | EC 1.13.11.44 linoleate dial synthase | |
| EC 1.13.11.7 deleted | EC 1.13.11.45 linoleate 1 1-lipoxygenase | EC 1 14.11.1 γ-butyrobetaine dioxygenase |
| EC 1.13.11.8 protocatechuate 4,5-dioxygenase | | EC 1.14.11.2 procollagen-proline dioxygenase |
| EC 1.13.11.9 2,5-dihydroxypyridine 5,6-dioxygenase | | EC 1.14.11.3 pyrimidine-deoxynucleoside 2′- |
| EC 1.13.11.10 7,8-dihydroxykynurenate 8,8a- | EC 1.13.12 | dioxygenase |
| dioxygenase | | EC 1 14.11.4 procollagen-lysine 5-dioxygenase |
| EC 1.13.11.11 tryptophan 2,3-dioxygenase | | EC 1.14.11.5 deleted, included in EC 1.14.11.6 |
| EC 1.13.11.12 lipoxygenase | WITH INCORPORATION OF ONE ATOM OF OXYGEN | EC 1.14.11.6 thymine dioxygenase |
| EC 1.13.11.13 ascarbate 2,3-dioxygenase | (INTERNAL MONOOXYGENASES OR INTERNAL MIXED | EC 1.14.11.7 procollagen-proline 3-dioxygenase |
| EC 1.13.11.14 2,3-dihydraxybenzoate 3,4-diaxygenase | FUNCTION OXIDASES) | EC 1.14.11.8 trimethyllysine dioxygenase |
| EC 1.13.11 15 3,4-dihydroxyphenylacetate 2,3- | | EC 1.14.11.9 naringenin 3-dioxygenase |
| dioxygenase | EC 1.13.12.1 arginine 2-monooxygenase | EC 1.14.11.10 pyrimidine-deoxynucleoside 1′- |
| EC 1.13.11.16 3-carboxyethylcatechol 2,3- | EC 1.13.12.2 lysine 2-monooxygenase | dioxygenase |
| dioxygenase | EC 1.13.12 3 tryptophan 2-monooxygenase | EC 1 14.11.11 hyoscyamine (6S)-dioxygenase |
| EC 1.13.11.17 indole 2,3-dioxygenase | EC 1.13.12.4 lactate 2-monooxygenase | EC 1.14.11.12 giberellin-44 dioxygenase |
| EC 1.13.11.18 sulfur dioxygenase | EC 1.13.12.5 Renilla-luciferin 2-monooxygenase | EC 1.14.11.13 giberellin 2β-dioxygenase |
| EC 1.13.11.19 cysteamine dioxygenase | EC 1.13.12.6 Cypridina-luciferin 2-monooxygenase | EC 1.14.11.14 6β-hydroxyhyoscyamine epoxidase |
| EC 1.13.11.20 cysteine dioxygenase | EC 1.13.12.7 Photmnus-luciferin 4-monooxygen | EC 1.14.11.15 bigerellin 3β-dioxygenase |
| EC 1.13.11.21 β-carotene 15,15-dioxygenase | (ATP-hydrolysing) | EC 1.14.11.16 peptide-aspartate β-dioxygenase |
| EC 1.13.11.22 caffeate 3,4-dioxygenase | EC 1.13.12.8 Watasenia-luciferin 2-manooxygenase | EC 1.14.11.17 taurine dioxygenase |
| EC 1.13.11.23 2,3-dihydroxyindole 2,3-dioxygenase | EC 1.13.12.9 phenylalanine 2-monooxygenase | EC 1.14.11.18 phytanoyl-CoA dioxygenase |
| EC 1.13.11.24 quercetin 2,3-dioxygenase | EC 1.13.12.10 lysine 6-monooxygenase | |
| EC 1.13.11.25 3,4-dihydroxy-9, 10-secoandrosta- | EC 1.13.12.11 methylphenyltetrahydropyridin N- | |
| 1,3,5(10)-triene-9, 1 7-dione 4,5-dioxygenase | monooxygenase | EC 1.14.12 |
| EC 1.13.11.26 peptide-tryptaphan 2,3-diaxygenase | EC 1.13.12.12 apo-β-carotenoid-14′,13′-dioxygenase | |
| EC 1.13.11.27 4-hydraxyphenylpyruvate dioxygenase | | |
| EC 1.13.11.28 2,3-dihydroxybenzoate 2,3-diaxygenase | | WITH AND OR NADH AS ONE DONOR, AND |
| EC 1.13.11.29 stizolobate synthase | EC 1.13.99 | INCORPORATION OF TWO ATOMS OF OXYGEN INTO |
| EC 1 13.11.30 stizolobinate synthase | | ONE DONOR |
| EC 1.13.11.31 arachidanate 12-lipoxygenase | | |
| EC 1.13.11.32 2-nitrapropane dioxygenase | MISCELLANEOUS | EC 1.14.12.1 anthranilate 1,2-dioxygenase |
| EC 1.13.11.33 arachidanate 15-lipaxygenase | | (deaminating, decarboxylating) |
| EC 1.13.11.34 arachidonate 5-lipoxygenase | EC 1.13.99.1 myo-inositol oxygenase | EC 1.14.12.2 now EC 1.14.13.35 |
| EC 1.13.11.35 pyrogallal 1,2-oxygenase | EC 1.13.99.2 now EC 1.14.12.10 | EC 1.14.12.3 benzene 1,2-dioxygenase |
| | EC 1.13.99.3 tryptophan 2′-dioxygenase | EC 1.14.12.4 3-hydroxy-3-methylpyridinecarboxylate |
| dioxygenase | EC 1.14.13.18 4-hydroxyphenylacetate 1- | EC 1.14.13.52 isoflavone 3′-hydroxylase |
| EC 1 14.12.5 5-pyridoxate dioxygenase | monooxygenase | EC 1.14.13.53 isoflavone 2′-hydroxylase |
| EC 1.14.12.6 now EC 1.14.13.66 | EC 1.14.13.19 taxifolin 8-monooxygenase | EC 1.14.13.54 ketosteroid monooxygenase |
| EC 1.14.12.7 phthalate 4,5-dioxygenase | EC 1.14.13.20 2,4-dichlorophenol 6-monooxygenase | EC 1.14.13.55 protopine 6-monooxygenase |
| EC 1.14.12.8 4-sulfobenzoate 3,4-dioxygenase | EC 1.14.13.56 dihydrosanguinarine 10- | |
| EC 1.14.12.9 4-chlorophenylacetate 3,4-dioxygenase | EC 1.14.13.21 flavonoid 3-monooxygenase | monooxygenase |
| EC 1.14.12.10 benzoate 1,2-dioxygenase | EC 1.14.13.22 cyclohexanone monooxygenase | EC 1.14.13.57 dihydrochelirubine 12-monooxygenase |
| EC 1.14.12.11 toluene dioxygenase | EC 1.14.13.23 3-hydroxybenzoate 4-monooxygenase | EC 1.14.13.58 benzoyl-CoA 3-monooxygenase |
| EC 1 14.12.12 naphthalene 1,2-dioxygenase | EC 1.14.13.24 3-hydroxybenzoate 6-monooxygenase | EC 1.14.13.59 L-lysine 6-monooxygenase (NADPH_{2 )} |
| EC 1.14.12.13 2-chlorobenzoate 1,2-dioxygenase | EC 1.14.13.25 methane monooxygenase | EC 1.14.13.60 27-hydroxycholesterol 7α- |
| EC 1.14.12.14 2-aminobenzenesulfonate 2,3- | EC 1.14.13.26 phosphatidylcholine 12- | monooxygenase |
| dioxygenase | monooxygenase | EC 1.14.13.61 2-hydroxyquinoline 8-monooxygenase |
| EC 1.14.12.15 terephthalate 1,2-dioxygenase | EC 1.14.13.27 4-aminobenzoate 1-monooxygenase | EC 1.14.13.62 4-hydroxyquinoline 3-monooxygenase |
| EC 1.14.12.16 2-hydroxyquinoline 5,6-dioxygenase | EC 1.14.13.28 3,9-dihydroxypterocarpan 6α- | EC 1.14.13.63 3-hydroxyphenylacetate 6-hydroxylase |
| EC 1 14.12.17 nitric oxide dioxygenase | monooxygenase | EC 1.14.13.64 4-hydroxybenzoate 1-hydroxylase |
| EC 1.14.12.18 biphenyl 2,3-dioxygenase | EC 1.14.13.29 4-nitrophenol 2-monooxygenase | EC 1.14.13.65 2-hydroxyquinoline 8-monooxygenase |
| | EC 1.14.13.30 leucotriene-B₄ 20-monooxygenase | EC 1.14.13.66 2-hydroxycyclohexanone 2- |
| | EC 1.14.13.31 2.nitrophenol 2-monooxygenase | monooxygenase |
| EC 1.14.13 | EC 1.14.13.32 albendazole monooxygenase | EC 1.14.13.67 quinone 3-monooxygenase |
| | EC 1.14.13.33 4-hydroxybenzoate 3-monooxygenase | EC 1.14.13.68 4-hydroxyphenylacetaldehyde oxime |
| | [AND(P)H_{2]} | monooxygenase |
| WITH AND OR NADH AS ONE DONOR, AND | EC 114.13.34 leucotriene-E₄ 20-monooxygenase | |
| INCORPORATION OF ONE ATOM OF OXYGEN | EC 1.14.13.35 anthranilate 3-monooxygenase | |
| | (deaminating) | EC 1.14.14 |
| EC 1.14.13.1 salicylate 1-monooxygenase | EC 1.14.13.36 5.O.(4-coumaroyl)-D-quinate 3′- | |
| EC 1.14.13.2 4-hydroxybenzoate 3-monooxygenase | monooxygenase | |
| EC 1.14.13.3 4-hydroxyphenylacetate 3- | EC 1.14.13.37 methyltetrahydroprotoberberine 14- | WITH REDUCED FLAVIN OR FLAVOPROTEIN AS ONE |
| monooxygenase | monooxygenase | DONOR, AND INCORPORATION OF ONE ATOM OF |
| EC 1.14.13.4 melilotate 3-monooxygenase | EC 1.14.13.38 anhydrotetracycline monooxygenase | OXYGEN |
| EC 1.14.13.5 imidazoleacetate 4-monooxygenase | EC 1.14.13.39 nitric-oxide synthase | |
| EC 1.14.13.6 orcinol 2-monooxygenase | EC 1.14.13.40 anthraniloyl-CoA monooxygenase | EC 1.14.14.1 unspecified monooxygenase |
| EC 1.14.13.7 phenol 2-monooxygenase | EC 1.14.13.41 tyrosine N-monooxygenase | EC 1.14.14.2 deleted, included in EC 1.14.14.1 |
| EC 1.14.13.8 dimethylaniline monooxygenase (N- | EC 1.14.13.42 hydroxyphenylacetonitrile 2- | EC 1.14.14.3 alkanol monooxygenase (FMN-linked) |
| oxide-forming) | monooxygenase | EC 1.14.14.4 choline monooxygenase |
| EC 1.14.13.9 kynurenine 3-monooxygenase | EC 1.14.13.43 questin monooxygenase | |
| EC 1.14.13.10 2,6-dihydroxypyridine 3- | EC 1.14.13.44 2-hydroxybiphenyl 3-monooxygenase | |
| monooxygenase | EC 1.14.13.45 CMP-N-acetylneuraminate | EC 1.14.15 |
| EC 1.14.13.11 trans-cinnamate 4-monooxygenase | monooxygenase | |
| EC 1.14.13.12 benzoate 4-monooxygenase | EC 1.14.13.46 (·)-menthol monooxygenase | |
| EC 1.14.13.13 calcidiol 1-monooxygenase | EC 1.14.13.47 (·)-limonene 3-monooxygenase | WITH REDUCED IRON-SULFUR PROTEIN AS ONE |
| EC 1.14.13.14 trans-cinnamate 2-monooxygenase | EC 1.14.13.48 (·)-limonene 6-monooxygenase | DONOR, AND INCORPORATION OF ONE ATOM OF |
| EC 1.14.13.15 cholestanetriol 26-monooxygenase | EC 1.14.13.49 (·)-limonene 7-monooxygenase | OXYGEN |
| EC 1.14.13.16 cyclopentanone monooxygenase | EC 1.14.13.50 pentachlorophenol monooxygenase | |
| EC 1.14.13.17 cholesterol 7α-monooxygenase | EC 1.14.13.51 6-oxocineole dehydrogenase | EC 1.14.15.1 camphor 5-monooxygenase |
| | EC 1.14.15.2 camphor 1,2-monooxygenase | |
| EC 1.14.15.3 alkane 1-monooxygenase | EC 1.14.99 | EC 1.14.99.35 thiophene-2-carbonyl-CoA |
| EC 1.14.15.4 steroid 11β-monooxygenase | | monooxygenase |
| EC 1.14.15.5 corticosterone 18-monooxygenase | | |
| EC 1.14.15.6 cholesterol monooxygenase (side-chain- | MISCELLANEOUS | |
| cleaving) | | EC 1.15.1 |
| EC 1.14.15.7 choline monooxygenase | EC 1.14.99 1 prostaglandin-endoperoxide synthase | EC 1.15 1.1 superoxide dismutase |
| | EC 1.14.99.2 kynurenine 7,8-hydroxylase | |
| | EC 1.14.99 3 heme oxygenase (decyclizing) | |
| EC 1.14.16 | EC 1.14.99.4 progesterone monooxygenase | EC 1.16.1 |
| | EC 1.14.99.5 now EC 1.14.19.1 | |
| | EC 1.14.99.6 now EC 1.14.19.2 | |
| WITH REDUCED PTERIDINE AS ONE DONOR, AND | EC 1.14.99.7 squalene monooxygenase | WITH AND OR NADP AS ACCEPTOR |
| INCORPORATION OF ONE ATOM OF OXYGEN | EC 1.14.99.8 deleted, included in EC 1.14.14.1 | |
| | EC 1.14.99.9 steroid 17α-monooxygenase | EC 1.16.1.1 mercury(II) reductase |
| EC 1 14.16.1 phenylalanine 4-monooxygenase | EC 1.14.99.10 steroid 21-monooxygenase | EC 1.16.1.2 diferric-transferrin reductase |
| EC 1.14.16.2 tyrosine 3-monooxygenase | EC 1.14.99.11 estradiol 6β-monooxygenase | |
| EC 1.14.16.3 anthranilate 3-monooxygenase | EC 1.14.99.12 4-androstene-3,17-dione | |
| EC 1.14.16.4 tryptophan 5-monooxygenase | monooxygenase | EC 1.16.3 |
| EC 1.14.16.5 glyceryl-ether monooxygenase | EC 1.14.99.13 now EC 1.14.13.23 | |
| EC 1.14.16.6 mandelate 4-monooxygenase | EC 1.14.99.14 progesterone 11α-monooxygenase | |
| | EC 1.14.99.15 4-methoxybenzoate monooxygenase | WITH OXYGEN AS ACCEPTOR |
| | (O-demethylating) | |
| EC 1.14.17 | EC 1.14.99.16 methylsterol monooxygenase | EC 1.16.3.1 ferroxidase |
| | EC 1.14.99.17 now EC 1.14.16.5 | |
| | EC 1.14.99.18 N-acetylneuraminate monooxygenase | |
| WITH REDUCED ASCORBATE AS ONE DONOR, AND | EC 1.14.99.19 plasmanylethanolamine desaturase | EC 1.17.1 |
| INCORPORATION OF ONE ATOM OF OXYGEN | EC 1.14.99.20 phylloquinone monooxygenase (2,3- | |
| | epoxidizing) | WITH AND OR NADP AS ACCEPTOR |
| EC 1.14.17.1 dopamine β-monooxygenase epoxidizing | EC 1.14.99.21 Latia-luciferin monooxygenase | |
| EC 1.17.1.1 CDP-4-dehydro-6-deoxyglucoase reductase | | |
| EC 1.14.17.2 deleted, included in EQ 1.14.18.1 | (demethylating) | |
| EC 1.14.17.3 peptidylglycine monooxygenase | (demethylating) | EC 1.14.99 22 ecdysone 20-monooxygenase |
| | EC 1.14.99.23 3-hydroxybenzoate 2-monooxygenase | EC 1.17.3 |
| | EC 1.14.99.24 steroid 9α-monooxygenase | |
| EC 1.14.18 | EC 1.14.99.25 now EC 1.14.19.3 | |
| | EC 1.14.99.26 2-hydroxypyridine 5-monooxygenase | WITH OXYGEN AS ACCEPTOR |
| | EC 1 14.99.27 juglone 3-monooxygenase | |
| WITH ANOTHER COMPOUND AS ONE DONOR, AND | EC 1.14.99.28 linalool 8-monooxygenase | EC 1.17.3 1 pteridine oxidase |
| INCORPORATION OF ONE ATOM OF OXYGEN | EC 1.14.99.29 deoxyhypusine monooxygenase | |
| | EC 1.14.99.30 carotene 7,8-desaturase | |
| EC 1.14.18.1 monophenol monooxygenase | EC 1.14.99.31 myristoyl-CoA 11-(E) desaturase | |
| | EC 1.14.99.32 myristoyl-CoA 11-(Z) desaturase | |
| | EC 1.14.99.33 Δ¹²-fatty acid dehydrogenase | |
| | EC 1.14.99.34 monoprenyl isoflavone epoxidase | |
| EC 1.17.4 | EC 1.19.6 | |
| WITH A DISULIDE AS ACCEPTOR | WITH DINITROGEN AS ACCEPTOR | |
| EC 1.17.4.1 ribonucleoside-diphosphate reductase | EC 1.19.6.1 nitrogenase (flavodoxin) | |
| EC 1.17.4.2 ribonucleoside-triphosphate reductase | | |
| | EC 1.97.1 | |
| EC 1.17.99 | EC 1.97.1.1 chlorate reductase | |
| | EC 1.97.1.2 pyrogallol hydroxyltransferase | |
| | EC 1.97.1.3 sulfur reductase | |
| WITH OTHER ACCEPTORS | EC 1.97.1.4 formate acetyltransferase activating | |
| | enzyme | |
| EC 1.17.99.1 4-cresol dehydrogenase (hydroxylating) | EC 1.97.1.5 arsenate reductase (glutaredoxin) | |
| | EC 1.97.1.6 arsenate reductase (donor) | |
| | EC 1.97.1.7 methylarsonate reductase | |
| 1.18.1 | | |
| WITH AND OR NADP AS ACCEPTOR | | |
| EC 1 18.1.1 rubredoxin-AND reductase | | |
| EC 1.18.1.2 ferredoxin-NADP reductase | | |
| EC 1.18.1.3 ferredoxin-AND reductase | | |
| EC 1.18.1.4 rubredoxin-AND(P) reductase | | |
| EC 1.18.6 | | |
| WITH DINITROGEN AS ACCEPTOR | | |
| EC 1.18.6.1 nitrogenase | | |
| EC 1.18.99 | | |
| WITH H* AS ACCEPTORS | | |
| EC 1.18 99 1 hydrogenase | | |

While the foregoing invention has been described in some detail for purposes of clarity and understanding, it will be clear to one skilled in the art from a reading of this disclosure that various changes in form and detail can be made without departing from the true scope of the invention. For example, all the techniques, methods, compositions, apparatus and systems described above may be used in various combinations. All publications, patents, patent applications, or other documents cited in this application are incorporated by reference in their entirety for all purposes to the same extent as if each individual publication, patent, patent application, or other document were individually indicated to be incorporated by reference for all purposes

What is claimed is:

1. A method for detecting one or more non-nucleic acid analytes, the method comprising:
(a) providing at least one fusion polypeptide with specificity for a non-nucleic acid analyte, which polypeptide comprises a first inactive functional domain; an analyte binding domain; and a second inactive functional domain;
wherein binding of the analyte results in a conformational change which brings the first inactive functional domain and the second inactive functional domain into proximity, thereby converting the first and second inactive functional domains into an optically detectable functional domain,
(b) contacting the fusion polypeptide with a sample comprising the analyte; and,
(c) detecting the conformational change induced by binding of the non-nucleic acid analyte, wherein the non-nucleic acid analyte is selected from the group consisting of a small organic molecule, a peptide, a polypeptide and a dissolved gas.

2. The method of claim 1, wherein the first and second inactive functional domains are derived from a green fluorescent protein or a green fluorescent protein homologue.

3. The method of claim 1, comprising detecting an electrochemical signal produced by binding of the analyte.

4. The method of claim 1, comprising detecting an optical signal produced by binding of the analyte.

5. The method of claim 4, wherein the optical signal is detected by one or more of: ultraviolet spectrophotometry, visible light spectrophotometry, surface plasmon resonance; calorimetry, fluorescence polarization; flurescence quenching; colorimetric quenching; fluroescence wavelength shift; fluroescence resonance energy transfer (FRET); enzyme linked immunosorbent assay (ELISA) or liquid crystal displays (LCD).

6. The method of claim 4, wherein the optical signal is produced by displacement of a tethered substrate upon binding of the analyte.

7. The method of claim 6, wherein the tethered substrate is an analyte analogue.

8. The method of claim 1, comprising providing a physical or logical array comprising a plurality of polypeptides.

9. The method of claim 8, wherein the plurality of polypeptides comprise a plurality of different analyte binding specificities.

10. The method of claim 10, wherein the plurality of polypeptides provide a common signal.

11. The method of claim 1, wherein the analyte comprises a hormone or a metabolite.

12. The method of claim 1, wherein the sample is a biological sample or an environmental sample.

13. The method of claim 12, wherein the biological sample is selected from the group consisting of blood, plasma, urine, sweat, cerebrospinal fluid and tears.

14. A method for detecting one or more non-nucleic acid analytes, the method comprising:
(a) providing at least one fusion polypeptide with specificity for a non-nucleic acid analyte, which polypeptide comprises a first inactive functional domain; an analyte binding domain; andn a second inactive functional domain;
wherein binding of the analyte results in a conformational change which brings the first inactive functional domain and the second inactive functional domain into proximity, therby converting the first and second inactive functional domains into a catalytic functional domain;
(b) providing a substrate for the catalytic functional domain;
(c) contacting the fusion polypeptide with a sample comprising the analyte; and,
(d) detecting the conversion of the substrate to a product.

15. The method of claim 14, wherein conversion of the substrate to a product is detected by detecting an electrochemical signal.

16. The method of claim 14, wherein conversion of the substrate to a product is detected by detecting an optical signal.

17. The method of claim 14, wherein the optical signal is detected by one or more of: ultraviolet spectrophotometry, visible light spectrophotometry, surface plasmon resonance; fluorescence polarization; fluorescence quenching; colorimetric quenching; fluorescence wavelength shift; fluorescence resonance energy transfer (FRET); enzyme linked immunosorbent assay (ELISA) or liquid crystal displays (LCD).

18. The method of claim 14, comprising providing a physical or logical array comprising a plurality of polypeptides.

19. The method of claim 18, wherein the plurality of polypeptides comprise a plurality of different analyte binding specificities.

20. The method of claim 19, wherein the plurality of polypeptides provide a common signal.

21. The method of claim 14, wherein the analyte comprises a small molecule.

22. The method of claim 14, wherein the small molecule comprises a hormone or a metabolite.

23. The method of claim 14, wherein the sample is a biological sample or an environmental sample.

24. The method of claim 23, wherein the biological sample is selected from the group consisting of blood, plasma, urine, sweat, cerebrospinal fluid and tears.

25. A method for detecting one or more non-nucleic acid analytes, the method comprising
(a) providing at least one polypeptide with specificity for a non-nucleic acid analyte, which polypeptide comprises an analyte binding domain and a catalytic domain, wherein binding of the analyte results in an allosteric conformational change which activates the catalytic domain resulting in conversion of a substrate to a detectable product; and,
(b) providing a substrate for the catalytic domain;
(c) contacting the polypeptide with a sample comprising the analyte; and,
(d) detecting the product produced by activity of the catalytic domain on the substrate.

26. The method of claim 25, wherein conversion of the substrate to product produces an electochemical signal.

27. The method of claim 25, wherein conversion of the substrate to product produces an optical signal.

28. The method of claim 27, wherein the optical signal is detected by one or more of: a charge coupled device, ultraviolet spectrophotometry, visible light spectrophotometry, fluorimetry, colorimetry, surface plasmon resonance; fluorescence polarization; flurescence quenching; colorimetric quenching; fluroescence wavelength shift; fluroescence resonance energy transfer (FRET); enzyme linked immunosorbent assay (ELISA) or liquid crystal displays (LCD).

29. The method of claim 25, comprising providing a physical or logical array comprising a plurality of polypeptides.

30. The method of claim 29, wherein the plurality of polypeptides comprise a plurality of different analyte binding specificities.

31. The method of claim 30, wherein conversion of substrate to product by the analyte-bound plurality of polypeptides is detected by detecting a common signal.

32. The method of claim 25, wherein the sample is a biological sample, an environmental sample, or an industrial sample.

33. The method of claim 32, wherein the biological sample is selected from the group consisting of blood, plasma, urine, sweat, cerebrospinal fluid and tears.

34. The method of claim 25, wherein the analyte comprises a small molecule.

35. The method of claim 25, wherein the analyte comprises a hormone, a metabolite or an ion.

36. The method of claim 25, wherein the analyte comprises an antigen or a ligand.

37. The method of claim 25, wherein the sample further comprises an agonist or an antagonist.

38. A method for detecting an analyte, the method comprising:
(a) providing at least one biopolymer, which biopolymer undergoes a conformational change upon binding to an analyte;
(b) contacting a sample comprising the analyte to the biopolymer; and,
(c) detecting the conformation change induced by binding of the analyte, wherein the analyte is not an ion.

39. The method of claim 38, comprising contacting a biological sample or an environmental sample.

40. The method of claim 39, wherein the biological sample comprises blood, plasma, urine, sweat, cerebrospinal fluid, or tears.

41. The method of claim 38, wherein the at least one biopolymer comprises a polypeptide.

42. The method of claim 41, wherein the polypeptide comprises an antibody or a receptor.

43. The method of claim 38, wherin the conformation change results in generation of an optical signal.

44. The method of claim 43, wherein the optical signal is detected by one or more of: surface plasmon resonance; fluorescence polarization; flurescence quenching; calorimetric quenching; fluroescence wavelength shift; fluroescence resonance energy transfer (FRET); enzyme linked immunosorbent assay (ELISA) or liquid crystal displays (LCD).

45. The method of claim 43, wherein the optical signal is produced by displacement of a tethered substrate upon binding of the analyte.

46. The method of claim 45, wherein the tethered substrate is an analyte analogue.

47. The method of claim 38, comprising providing a physical or logical array comprising a plurality of polypeptides.

48. The method of claim 47, wherein the plurality of polypeptides comprise a plurality of different analyte binding specificities.

49. The method of claim 48, wherein the plurality of polypeptides provide a common signal.

50. The method of claim 38, wherein the analyte comprises a small molecule.

51. The method of claim 38, wherein the analyte comprises a hormone or a metabolite.

52. A method for identifying a physiologic state, the method comprising:
(a) providing at least one biopolymer, which biopolymer undergoes a conformational change upon binding to a marker associated with a physiologic state;
(b) contacting the biopolymer with a biological sample comprising the marker, and,
(c) detecting the conformation change induced by binding of the marker, thereby identifying the physiologic state associated with the marker.

53. The method of claim 53, wherein the sample comprises a biological sample selected from among: blood, plasma, urine, sweat, cerebrospinal fluid, or tears.

54. The method of claim 53, wherein the at least one biopolymer comprises a polypeptide.

55. The method of claim 54, wherein the polypeptide comprises an enzyme, an antibody, a receptor or a fusion protein.

56. The method of claim 54, wherein the polypeptide comprises a fusion protein having a first inactive functional domain; an analyte binding domain; and a second inactive functional domain.

57. The method of claim 56, wherein binding of the analyte results in a conformational change which brings the first inactive functional domain and the second inactive functional domain into proximity, thereby converting the first and second inactive functional domains into a functional catalytic or fluorescent domain.

58. The method of claim 57, wherin the conformation change results in generation of an optical signal.

59. The method of claim 52, wherein the optical signal is detected by one or more of: ultraviolet spectrophotometry, visible light spectrophotometry, surface plasmon resonance; fluorescence polarization; flurescence quenching; colorimetric quenching; fluroescence wavelength shift; fluroescence resonance energy transfer (FRET); enzyme linked immunosorbent assay (ELISA) or liquid crystal displays (LCD).

60. The method of claim 52, wherein the optical signal is produced by displacement of a tethered substrate upon binding of the analyte.

61. The method of claim 60, wherein the tethered substrate is an analyte analogue.

62. The method of claim 52, comprising providing a physical or logical array comprising a plurality of polypeptides.

63. The method of claim 62, wherein the plurality of polypeptides comprise a plurality of different analyte binding specificities.

64. The method of claim 63, wherein the plurality of polypeptides provide a common signal.

65. The method of claim 52, wherein the analyte comprises a small molecule.

66. The method of claim 52, wherein the analyte comprises a hormone or a metabolite.

67. A biosensor comprising:
(a) a support; and,
(b) at least one polypeptide with specificity for a non-nucleic acid analyte, which polypeptide comprises an analyte binding domain and a catalytic domain, wherein binding of the analyte results in an allosteric conformational change which activates the catalytic domain resulting in conversion of a substrate to a detectable product; which at least one polypeptide is immobilized on the support.

68. A biosensor comprising:
(a) a support; and,
(b) at least one fusion polyeptide with specificity for a non-nucleic acid analyte, which polypeptide comprises a first inactive functional domain; and analyte binding domain; and a second inactive functional domain; wherein binding of the analyte brings the first inactive functional domain and the second inactive functional domain into proximity, thereby converting the first and second inactive functional domains into a functional catalytic or optically detectable domain; which at least one fusion polypeptide is immobilized on the support.

69. A biosensor comprising:
(a) a solid support;
(b) a plurality of polypeptides immobilized on the solid support, wherein the plurality comprises polypeptides having different analyte binding specificities; and,
(c) a detection system.

70. The biosensor of claim 69, further comprising a conductive element or an optically detectable element.

71. The biosensor of claim 69, wherein the plurality of polypeptides is immobilized with an immobilization matrix selected from the group consisting of carbon paste and a non-biological polymeric matrix.

72. The biosensor of claim 69, wherein the biosensor further comprises a display.

73. A method of sensing one or more test stimulus, the method comprising:
providing a library of biopolymers comprising nucleic acid variants or expression products of the nucleic acid variants;
arraying the library in a spatial or logical format to provide a physical or logical array;
contacting one or more calibrating stimulus to the array, whereby one or more members of the array produce one or more detectable signals in response to contact by the one or more calibrating stimulus, thereby producing a calibrating array pattern which identifies contact of the array by the one or more calibrating stimulus;
contacting at least one test stimulus to the array, thereby producing a test stimulus array pattern; and,
comparing the test stimulus array pattern to the calibrating array pattern, thereby identifying the test stimulus.

74. A method of using a re-usable array of biopolymers, the method comprising:
providing a physical or logical array of biopolymers comprising nucleic acid variants or expression products of the nucleic acid variants;
contacting the physical or logical array with one or more first stimulus;
observing a first resulting response of the array, or collecting a first product resulting from contact between the array and the first stimulus;
reusing the array by contacting the array a second time with the first stimulus, or with a second stimulus; and,
observing a second resulting response of the array, or collecting a second product resulting from contact between the array and the first or second stimulus; and, optionally, comparing the first resulting response of the array to the second resulting response of the array.

75. The method of claim 73 or 74, wherein the biopolymer library comprises or is encoded by recursively recombined nucleic acids.

76. The method of claim 73 or 74, wherein the biopolymer library comprises or is encoded by artificially mutated or artificially shuffled nucleic acids.

77. The method of claim 73 or 74, wherein the biopolymer library comprises or is encoded by species variants of one or more nucleic acids.

78. The method of claim 73 or 74, wherein the biopolymer library comprises or is encoded by nucleic acids produced by recursive recombination of species variants of one or more nucleic acids.

79. The method of claim 73 or 74, wherein the biopolymer library comprises photoactivatable members.

80. The method of claim 79, the method comprising masking a portion of the array and exposing the resulting masked array to light.

81. The method of claim 73 or 74, wherein the array comprises one or more of: a conductive member, a capacitive member, an optically responsive member, an electrically responsive member, and an electrically or logically gated or gateable member.

82. The method of claim 73 or 74, wherein the array comprises one or more of: a bio-laser, a polychromic display, a molecular poster, a bar code, a protein TV, a molecular camera, a UV molecular camera, an IR molecular camera, or a flat screen display.

83. The method of claim 73 or 74, wherein the array members comprise one or more proteins.

84. The method of claim 83, wherein the proteins comprise electrically conductive proteins.

85. The method of claim 83, wherein the proteins are purified.

86. The method of claim 83, wherein the proteins comprise one or more purification tags.

87. The method of claim 86, wherein the purification tags are selected from the group consisting of: His tags, and FLAG tags.

88. The method of claim 73 or 74, wherein arraying the biopolymer library comprises arranging the members of the library in a logically accessible format.

89. The method of claim 73 or 74, wherein arraying the biopolymer library comprises arranging the members of the library in a physically gridded format.

90. The method of claim 73 or 74, wherein arraying the biopolymer library comprises plating the members of the library in microtiter trays.

91. The method of claim 73 or 74, wherein arraying the biopolymer library or expression product library comprises recording the position of members of the library in one or more database.

92. The method of claim 73 or 74, wherein arraying biopolymer library comprises arranging the members of the library for parallel examination.

93. The method of claim 73 or 74, wherein arraying the biopolymer library or expression product library comprises arranging the members of the library for sequential examination.

94. The method of claim 73 or 74, wherein the first, second, test or calibrating stimulus are simultaneously contacted to a plurality of biopolymer library members.

95. The method of claim 73 or 74, wherein the first, second, test or calibrating stimulus are sequentially contacted to a plurality of biopolymer library members.

96. The method of claim 73 or 74, wherein a plurality of first, second, test or calibrating stimulus are contacted to a plurality of biopolymer members.

97. The method of claim 96, wherein contact of the plurality of first, second, test or calibrating stimulus produces a signature for a sample type.

98. The method of claim 97, wherein the signature is representative of one or more phenomenon selected from: a metabolic state of a cell, an operon induction in or by a cell, an induction of cell growth, a proliferation in or caused by a cell, a cancer of a cell or tissue, or organism, apoptosis, cell death, cell cycle, cell or tissue differentiation, tumorigenesis, disease state, drug resistance, drug efficacy, antibiotic spectrum, drug toxicity, gas level, SOₓ, NOₓ Alzheimers disease, infection, presence of viruses, viral infection, bacterial infection, HIV infection, AIDS, serum cholesterol, CHDL level, LDL, serum triglyceride level, blood glucose level, ion or gas production or internalization, cytokine receptor expression, antibody-antigen interactions, pregnancy, fertility, fecundity, presence or absence of narcotics or other controlled substances, heart attack, presence or absence of steroids, body temperature, presence of sound waves, taste, scent, food composition, beverage composition, and an environmentally monitored condition.

99. The method of claim 73 or 74, wherein the first, second, test or calibrating stimulus are contacted to a plurality of library members in a microtiter plate.

100. The method of claim 73 or 74, wherein the first, second, test or calibrating stimulus are contacted to a plurality of library members fixed on a solid substrate.

101. The method of claim 73 or 74, wherein the first, second, test or calibrating stimulus are contacted to a plurality of library members, or expression products thereof, fixed on a solid substrate, wherein the solid substrate comprises a Nickel-NTA coated surface, a silane-treated surface, a pegylated surface, or a treated surface.

102. The method of claim 73 or 74, wherein the biopolymer library members or expression products thereof are fixed to an organizational matrix in spatially addressable locations.

103. The method of claim 73 or 74, wherein the first, second, test or calibrating stimulus are contacted to a plurality of biopolymer library members, wherein member types are fixed on the surface of one or more beads.

104. The method of claim 103, wherein the one or more beads each comprise more than one detectable feature.

105. The method of claim 104, wherein the more than one detectable feature includes a first feature which identifies binding by the first, second, test or calibrating stimulus and a second feature which identifies either the type of bead or the type of library member or expression product thereof which is bound to the bead.

106. The method of claim 73 or 74, wherein the first stimulus, the second stimulus, the calibrating stimulus or the test stimulus, is selected from the group consisting of: light, radiation, an atom, an ion, and a molecule.

107. The method of claim 73 or 74, wherein the first, second, test or calibrating stimulus comprises, hybridizes to, binds, acts upon or is acted upon by one or more of: radiation, a polymer, a chemical moiety, a biopolymer, a nucleic acid, an RNA, a DNA, a protein, a ligand, an enzyme, a chemo-specific enzyme, a regio-specific enzyme, a stereo-specific enzyme, a nuclease, a restriction enzyme, an restriction enzyme which recognizes a triplet repeat, a restriction enzyme that recognizes DNA superstructure, a restriction enzyme with an 8 base recognition sequence, an enzyme substrate, a regio-specific enzyme substrate, a stereo-specific enzyme substrate, a ligase, a thermostable ligase, a polymerase, a thermostable polymerase, a co-factor, a lipase, a protease, a glycosidase, a toxin, a contaminant, a metal, a heavy metal, an immunogen, an antibody, a disease marker, a cell, a tumor cell, a tissue-type, cerebro-spinal fluid, a cytokine, a receptor, a chemical agent, a biological agent, a fragrance, a pheromone, a hormone, an olfactory protein, a metabolite, a molecular camera protein, a rod protein, a cone protein, a light-sensitive protein, a lipid, a pegylated material, an adhesion amplifier, a drug, a potential drug, a lead compound, a protein allele, an oxidase, a reductase, or a catalyst.

108. The method of claim 73 or 74, wherein the first, second, test or calibrating stimulus are contacted to the members of the library by incubating a solution comprising the test molecule or the calibrating molecule with the library members.

109. The method of claim 108, wherein the solution is a fluid, a polymer solution or a gel.

110. The method of claim 73 or 74, wherein comparison of the test array pattern and the calibrating array pattern, or of the first resulting response of the array and the second resulting response of the array, is performed by a computer.

111. The method of claim 73 or 74, wherein a plurality of first, second, test or calibrating stimuli are contacted to the array to produce a plurality of resulting array patterns.

112. The method of claim 111, further comprising recording the plurality of resulting array patterns in one or more databases.

113. The method of claim 112, further comprising assigning a bar code to each resulting array pattern.

114. The method of claim 73 or 74, wherein the test array pattern, the calibrating array pattern, the first resulting response of the array, or the second resulting response of the array, comprises variations in the presence or absence of signal at different locations on or in the array.

115. The method of claim 73 or 74, wherein the test array pattern, the calibrating array pattern, the first resulting response of the array, or the second resulting response of the array comprises variations in the level of signal at different locations on the array.

116. The method of claim 73 or 74, wherein the test array pattern, the calibrating array pattern, the first resulting response of the array, or the second resulting response of the array comprises variations in the presence and intensity of signal at different locations on the array.

117. The method of claim 73 or 74, wherein an intensity of the test array pattern, the calibrating array pattern, the first resulting response of the array, or the second resulting response of the array comprises is measured to quantify the first, second, test or calibrating stimulus.

118. The method of claim 73 or 74, wherein the test array pattern, the calibrating array pattern, the first resulting response of the array, or the second resulting response of the array comprises one or more fluorophore emission, photon emission, chemiluminescent emission, coupled luminescent/fluorescent emission or quenching, or detection of one or more fluorophore emission.

119. The method of claim 73 or 74, wherein the test array pattern, the calibrating array pattern, the first resulting response of the array, or the second resulting response of the array comprises an electorchemally detectable signal, an amperometrically detectable signal, a potentiometrically detectable signal, a signal detectable as a change in pH, a signal based on specific ion levels, a signal based on changes in conductivity, a pizoelectric signal, a change in resonance frequency, a signal detectable as surface accoustic waves, or a signal detectable by quartz crystal microbalances.

120. The method of claim 118, wherein the test array pattern, the calibrating array pattern, the first resulting response of the array or the second resulting response of the array comprises multiple wavelengths of light.

121. The method of claim 118, wherein the test array pattern, the calibrating array pattern, the first resulting response of the array or the second resulting response of the array is generated by detection of one or more of: light, H₂O₂, glucose oxidase, NADP, NADPH⁺, NAD(P)H reductase, a change in reduction potential, a change in protein conformation, a change in intrinsic fluorescence, fluorescence, luminescence, FRET, absorbtion, surface plasmon resonance, antigen binding, antibody binding, enzyme activity, opening of an ion channel, or label binding.

122. The method of claim 73 or 74, wherein at least one member of the biopolymer library, or an expression product thereof, is selected, prior to the arraying step, for one or more of: enhanced stability, orientation of protein binding, improved production, cost of manufacture, optimal activity of expressed members which comprise a tag, overexpression mutations, optimized protein folding, permanent enzyme secretion, improved operators, improved ribosome binding sites, avidity, selectivity, production of a detectable side product, and detection limit.

123. The method of claim 73 or 74, wherein the test array pattern, the calibrating array pattern, the first resulting response of the array or the second resulting response of the array are detected by one or more of: a microscope, a CCD, a phototube, a photodiode, an LCD, a scintillation counter, film, or visual inspection.

124. The method of claim 73 or 74, wherein the test array pattern, the calibrating array pattern, the first resulting response of the array or the second resulting response of the array are digitized and stored in one or more database in one or more computer.

125. The biopolymer array produced by the method of claim 73 or 74.

126. The biopolymer array of claim 125, wherein the array is stable for at least one year under pre-selected storage conditions.

127. A computer comprising a data set corresponding to the labeling biopolymer sensor array pattern or test biopolymer sensor array pattern of claim 73 or 74.

128. The method of claim 73 or 74, further comprising contacting at least one additional stimulus to the array, and comparing a resulting additional test stimulus array pattern to the calibrating array pattern, thereby identifying the at least one additional stimulus, or observing an additional resulting response of the array, or collecting an additional product resulting from contact between the array and the additional or a previous stimulus, and optionally comparing the additional resulting response to any one or more previous responses of the array.

129. The method of claim 128, comprising contacting the array with two or more additional stimuli.

130. The method of claim 129, comprising contacting 10 or more additional stimuli to the array.
